(19) United States
(12) Patent Application Publication
(10) Pub. No.: US 2020/0138854 A1
(43) Pub. Date: May 7, 2020

(54) Method of Detecting and Quantifying Bile Acid from Saliva

(71) Applicant: Ironwood Pharmaceuticals, Inc., Boston, MA (US)

(72) Inventors: Mark G. Currie, Boston, MA (US); Nisha Lizan Perez, Malden, MA (US)

(21) Appl. No.: 16/631,214

(22) PCT Filed: Jul. 19, 2018
(86) PCT No.: PCT/US2018/042881

§ 371 Date: Jan. 15, 2020

Related U.S. Application Data
(60) Provisional application No. 62/534,591, filed on Jul. 19, 2017.
(60) Provisional application No. 62/681,633, filed on Jun. 6, 2018.

(51) Int. Cl. A61K 31/785; A61K 45/06; A61K 9/28; A61K 9/20; A61P 1/04

(52) U.S. Cl. CPC A61K 31/785; A61K 45/06; A61P 1/04; A61K 9/2031; A61K 9/28

(57) ABSTRACT

A method of detecting and quantifying bile acids from saliva from a human patient; the method involves collecting saliva from the patient and determining the bile acid levels in the saliva, using, for example, liquid chromatography with tandem mass spectrometry. The human patient may be treated with an enteric coated gastro-retentive oral dosage form in the form of a tablet of a bile acid sequestrant dispersed in a polymeric matrix.

### TECHNICAL FIELD

This disclosure relates, inter alia, to methods of detecting and quantifying bile acids from saliva.

### BACKGROUND

Methods for detecting bile acids from subjects suffer from major drawbacks. For example, the Bilitec® ambulatory bile reflux monitor actually detects bilirubin as a surrogate of bile acids (Barrett et al., (2000) Dis. Esophagus, 13, 44-50), and thus cannot be used to quantitate the levels of different bile acids. The Bilitec® assay is a disruptive procedure requiring placing a tube through the nose into the esophagus of a subject; cannot be used to detect low levels of bile acids; and has only a marginal correlation to bile acid levels (Barrett et al., id.). There is, therefore, a need for a non-invasive method for testing for the presence and quantification of individual bile acid levels.

### SUMMARY

This disclosure provides assays for the detection and quantitation of bile acids from saliva. The method can be used, for example, in the identification of subjects that may be receptive to the therapeutic compositions and methods described herein. The assay can also be used to monitor the progress of the therapies described herein.

Numerous other aspects are provided in accordance with these and other aspects of the invention. Other features and aspects of the present invention will become more fully apparent from the following detailed description and the appended claims.

[E01] According to a first aspect of the invention, there is provided, a method for detecting and quantifying bile acids from saliva from a human patient, comprising: collecting saliva from said patient and determining the bile acid levels in the saliva using liquid chromatography with tandem mass spectrometry.

[E02] The method of E01, wherein the human patient is being treated with an enteric coated gastro-retentive oral dosage form in the form of a tablet of a bile acid sequestrant dispersed in a polymeric matrix.

[E03] The method of E02, wherein the polymeric matrix comprises polyethylene oxide (CAS Number 25322-68-3, approximate molecular weight 300,000 (PEG-7M)).

[E04] The method of any one of E01-E03, wherein the dosage form further comprises one or more filler or compressing agent.

[E05] The method of E04, wherein the one or more filler or compressing agent is selected from microcrystalline cellulose, butylated hydroxytoluene, colloidal silicon dioxide, lactose, starch, maltodextrins, magnesium stearate, diacetylated monoglycerides, hypromellose, and dibasic calcium phosphate.

[E06] The method of any one of E01-E05, wherein the tablet is coated with an enteric coating.

[E07] The method of any one of E01-E06, further comprising administering a pharmaceutical composition comprising a proton pump inhibitor (PPI).

[E08] The method of any one of E01-E07, wherein the bile acid sequestrant is colesevelam or colesevelam hydrochloride.

[E09] The method of any one of E01-E08, wherein the patient is administered a dose of 500 mg, 700 mg, 750 mg, 1,000 mg, 1400 mg, 1,500 mg, or 2,100 mg, or more, of the bile acid sequestrant, twice per day.

[E10] The method of any one of E01-E09, wherein the patient is administered a dose is 1,500 mg, twice per day. [E11] The method of E10, wherein the dose of 1,500 mg is administered as either 2 tablets, each tablet having 750 mg of the bile acid sequestrant or as 3 tablets, each tablet having 500 mg of the bile acid sequestrant, twice per day.

[E12] The method of any one of E01-E11, wherein the saliva sample has a concentration of total bile acids exceeding 50 μmol/L.

[E13] The method of any one of E01-E12, wherein the saliva sample has a concentration of total bile acids exceeding 13 nM.

[E14] The method of any one of E01-E13, wherein the saliva sample has a concentration of total bile acids exceeding 37 nM.

[E15] A method of monitoring progress of GERD, wherein samples of a subject who is being given a bile acid lowering or sequestering agent is monitored, and a reduction in bile acid levels is indicative of effective therapy.

### BRIEF DESCRIPTION OF THE DRAWINGS

FIG. 1 shows the LC/MS/MS profile of an internal standard of ‘spiked’ bile acids.

FIG. 2 shows a standard curve for the quantitation of GCA.

FIG. 3 shows the level of total conjugated bile acids in normal subjects as well as GERD patients on PPI therapy before and after a hearty meal.

FIG. 4 shows graphically when sampling saliva ≤2 hrs post meal, correlation is seen between saliva bile acid positive and Bilitec positive results. FIG. 4a shows ROC Curve for all 45 Subjects with Bilitec: AUC=0.47. FIG. 4b shows ROC Curve for 10 Subjects with saliva sampling ≤2 hrs post meal: AUC=0.88.

FIG. 5 shows that using saliva samples collected ≤2 hrs post meal, the threshold to reach 100% sensitivity is 13 nM.

FIG. 6 shows that using saliva samples collected ≤2 hrs post meal, the threshold to reach 100% specificity is 37 nM.

FIG. 7 shows efficacy results in saliva bile positive subgroup compared with overall population. FIG. 7, left panel: % Chg from BL to W8 in WHSS; Overall Population: Diff=6.7. FIG. 7, right panel: % Chg from BL to W8 in WRFS; Overall Population: Diff=14.4.

FIG. 8 shows when sampling saliva ≤2 hrs post meal, correlation is seen between saliva bile DCA positive and Bilitec positive results, similar trend to total bile acids. FIG. 8a: ROC Curve for all 45 Subjects with Bilitec: AUC=0.47. FIG. 8b: ROC Curve for 10 Subjects with saliva sampling ≤2 hrs post meal: AUC=0.74.

FIG. 9 shows that using saliva samples collected ≤2 hrs post meal, the DCA threshold to reach 80% sensitivity/specificity is 2 nM.

FIG. 10 shows efficacy results in saliva bile DCA positive subgroup compared with overall population. FIG. 10a: % Chg from BL to W8 in WRFS; Overall Population: Diff=14.4. FIG. 10b: % Chg from BL to W8 in WHSS; Overall Population: Diff=6.7.

### DETAILED DESCRIPTION

As used herein, the word “a” or “plurality” before a noun represents one or more of the particular noun.

As used herein, the term “subject” and “patient” are used interchangeably. A patient or a subject may be a human patient or a human subject.

The term “PEG-7M” used herein refers to polyethylene oxide CAS Number 25322-68-3, approximate molecular weight 300,000 (PEG-7M) (Polyox™ WSR N-750). The terms “Polyox™ WSR N-750” and “PEG-7M,” both refer to polyethylene oxide CAS Number 25322-68-3, approximate molecular weight 300,000.

The term “gastro-retentive dosage form” denotes dosage forms which are designed to be retained in the upper gastrointestinal tract for a prolonged period of time (generally, at least 4 hours) during which they release the drug on a controlled basis.

For the terms “for example” and “such as,” and grammatical equivalences thereof, the phrase “and without limitation” is understood to follow unless explicitly stated otherwise. As used herein, the term “about” is meant to account for variations due to experimental error. All measurements reported herein are understood to be modified by the term “about,” whether or not the term is explicitly used, unless explicitly stated otherwise. As used herein, the singular forms “a,” “an,” and “the” include plural referents unless the context clearly dictates otherwise.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Bile Acids

Bile reflux occurs when bile, a digestive fluid produced in the liver, flows upward (refluxes) from the small intestine into the stomach and then into the esophagus. Bile reflux often accompanies acid reflux, and together they may cause inflammation of the esophageal lining and potentially increased risk of esophageal cancer. See AJG (1999) 94(12):3649-3650. Bile reflux may also affect the stomach, causing inflammation (gastritis, which, if untreated, can lead to peptic ulcers). Bile reflux can be difficult to distinguish from acid reflux because the signs and symptoms are similar, and the two conditions frequently occur at the same time. Unlike acid reflux, bile reflux inflames the stomach, often causing a gnawing or burning pain in the upper abdomen. Other signs and symptoms may include: frequent heartburn, i.e., a burning sensation in the chest that sometimes spreads to the throat along with a sour taste in the mouth; nausea; vomiting bile; a cough; or hoarseness.

Bile acids are steroid acids found predominantly in the bile of mammals. They are produced in the liver by the oxidation of cholesterol and they and are stored in gallbladder and secreted into the intestine in the form of salts. They act as surfactants, emulsifying lipids and assisting with the absorption and digestion of dietary fat and cholesterol. The principal bile acids are: cholic acid, chenodeoxycholic acid, deoxycholic acid, taurocholic acid, and glycocholic acid. The chemical distinctions between different bile acids are small, depending only on the presence or absence of hydroxyl groups on positions 3, 7, and 12. In humans, the most prevalent bile acids are cholic acid and chenodeoxycholic acid, and their conjugates with taurine and glycine (glycocholate and taurocholate). Some mammals synthesize predominantly deoxycholic acid.

Bile acids play an important role in the digestive process. However, the prolonged presence or excess of bile acids in the stomach and esophagus can result in toxic effects on regional tissues. Disorders and/or symptoms that are believed to be associated with bile reflux, either alone or in combination with acid reflux, include, for instance, heartburn, indigestion, dyspepsia, erosive esophagitis, peptic ulcer, gastric ulcer, esophageal ulcers, esophagitis, laryngitis, pharyngitis, coarse or hoarse voice, and GERD-related pulmonary dysfunction such as coughing and/or asthma. Further complications that are believed to occur as a result of chronic bile reflux are, for instance, gastroesophageal reflux disease, or GERD; Barrett's esophagus; esophageal cancer (e.g., adenocarcinoma) and gastritis.

GERD is a generic term encompassing diseases with various digestive symptoms such as pyrosis, acid regurgitation, obstructed admiration, aphagia, pectoralgia, permeating feeling and the like sensibility caused by reflux in the esophagus and stagnation of gastric contents, duodenal juice, pancreatic juice and the like. The term covers both reflux esophagitis in which erosion and ulcers are endoscopically observed, and esophageal regurgitation-type non-ulcer dyspepsia (NUD) in which no abnormality is endoscopically observed. GERD occurs when the LES does not close properly and stomach contents leak back, or reflux, into the esophagus. A persistent GERD patient is a patient who does not respond to PPI.

A hiatal hernia may contribute to causing GERD and can happen in people of any age. Other factors that may contribute to GERD include, but are not limited to, alcohol use, being overweight, pregnancy, smoking, Zollinger-Ellison syndrome, hypercalcemia, and scleroderma. Also, certain foods can be associated with reflux events, including, citrus fruits, chocolate, drinks with caffeine, fatty and fried foods, garlic and onions, mint flavorings, spicy foods, and tomato-based foods, like spaghetti sauce, chili, and pizza.

The inner mucosa of the esophagus is lined with non-keratinized stratified squamous epithelium arranged in longitudinal folds. Damage to the lining of the esophagus causes the normal squamous cells lining the esophagus to turn into a type of cell not usually found in humans, called specialized columnar cells. That conversion of cells in the esophagus by the acid reflux is known as Barrett's Esophagus. Although people who do not have heartburn can have Barrett's esophagus, it is found about three to five times more often in people with this condition. Barrett's esophagus does not cause symptoms itself and is important only because it seems to precede the development of a particular kind of cancer—esophageal adenocarcinoma. The risk of developing adenocarcinoma is 30 to 125 times higher in people who have Barrett's esophagus than in people who do not. This type of cancer is increasing rapidly in white men. This increase may be related to the rise in obesity and GERD.

Barrett's esophagus has no cure, short of surgical removal of the esophagus, which is a serious operation. Surgery is recommended only for people who have a high risk of developing cancer or who already have it. Most physicians recommend treating GERD with acid-blocking drugs, since this is sometimes associated with improvement in the extent of the Barrett's tissue. However, this approach has not been proven to reduce the risk of cancer. Treating reflux with a surgical procedure for GERD also does not seem to cure Barrett's esophagus. Several different experimental approaches are under study. One attempts to see whether destroying the Barrett's tissue by heat or other means through an endoscope can eliminate the condition. This approach, however, has potential risks and unknown effectiveness.

Esophageal cancer can occur almost anywhere along the length of the esophagus, but it frequently starts in the glandular cells closest to the stomach (adenocarcinoma). Because esophageal cancer may not be diagnosed until it's quite advanced, the outlook for people with the disease is often poor. The risk of cancer of the esophagus is increased by long-term irritation of the esophagus, such as with smoking, heavy alcohol intake, and Barrett's esophagitis. Thus, there is a link between esophageal cancer and bile reflux and acid reflux. In animal models, bile reflux alone has been shown to cause cancer of the esophagus.

Unlike acid reflux, bile reflux usually cannot be controlled by changes in diet or lifestyle. Instead, bile reflux is most often managed with certain medications or, in severe cases, with surgery. Neither solution is uniformly effective, however, and some people continue to experience bile reflux even after treatment.

Numerous medications are used to treat heartburn and indigestion. Presently, the main therapies employed in the treatment of GERD and upper GI tract disorders include agents for reducing the stomach acidity, such as by using the histamine H2-receptor antagonists or proton pump inhibitors (PPIs). H2 blockers are drugs that inhibit the production of acid in the stomach. Exemplary histamine H2-receptor antagonists include, for example, cimetidine (as sold under the brand-name TAGAMET HB®), famotidine (as sold under the brand-name PEPCID AC®), nizatidine (as sold under the brand-name AXID AR®), and ranitidine (as sold under the brand-name ZANTAC 75®). Both types of medication are effective in treating heartburn caused by acid reflux and usually eliminate symptoms within a short period of time.

PPIs act by inhibiting the parietal cell H⁺/K⁺ ATPase proton pumps responsible for acid secretion from these cells. PPIs, such as omeprazole and its pharmaceutically acceptable salts are disclosed, for example, in EP 05129, EP 124495 and U.S. Pat. No. 4,255,431.

Despite their well-documented efficacy, PPIs have notable limitations. For example, patients who are non-responsive to treatment with PPI inhibitor alone may be non-responsive because even though the PPI is decreasing acid reflux from the stomach, bile acid from the duodenum is still present. Also, some patients with GERD are not fully responsive.

Method of Detecting Bile Acids and Associated Methods

This disclosure provides methods for the detection and quantitation of bile acids from fluid samples from a patient, including a human patient. The fluid samples can include samples taken from a subject, including urine, saliva, esophageal aspirations, serum or the like. As described herein below in the Examples, the method provides a highly sensitive, non-invasive assay to detect and quantitate bile acid levels in saliva. In certain embodiments, elevated bile acid levels in the saliva of a subject is associated with bile acid reflux and, therefore, indicates that the subject may be amenable to therapy using a bile acid sequestrant composition.

Therefore, in a first aspect, a method for the identification of patients receptive to the therapeutic compositions is disclosed. The method comprises collecting saliva, then quantitating the bile acid levels in the saliva, and determining whether the subject has an elevated bile acid level in the saliva. Generally, a sample is deemed to have an elevated bile acid level when the concentration of total bile acids exceeds 50 μmol/L, for example, at least 75 μmol/L, at least 100 μmol/L, at least 150 μmol/L, at least 200 μmol/L, at least 250 μmol/L, at least 300 μmol/L, or higher. In some embodiments, a sample is deemed to have an elevated bile acid level when the concentration of total bile acids exceeds 13 nM. In some embodiments, a sample is deemed to have an elevated bile acid level when the concentration of total bile acids exceeds 37 nM. In some embodiments, saliva samples are taken at least 2 hours after the subject has had the last meal, to eliminate the spike in bile acid levels that shortly follows a meal. But in some embodiments, saliva samples are taken within 2 hours after the subject has had the last meal. Subjects thus identified to have an elevated level of total bile acids are then administered with the gastric-retentive bile acid sequestrant composition.

In another aspect, therapeutic progress can be monitored using the detection methods described herein. The method comprises obtaining saliva samples from a subject who is being treated using a gastric-retentive bile acid sequestrant composition (or other agents for treating GERD) and determining the bile acid levels in the saliva samples. In some embodiments, a reduction in total bile acid levels as the course of therapy progresses is an indication of successful reduction in bile acid reflux. In other embodiments, the total bile acid levels are monitored during the course of therapy to determine whether the levels fall below a threshold level, which serves as an indication of successful therapy. Previous studies measuring bile acid levels in esophageal aspirations indicated that subjects with erosive esophagitis and Barrett's esophagus had significantly elevated bile acid (BA) levels (see, for example, Kauer et al. (1997) Surgery, 122, 874-881; and Nehra et al. (1999) Gut, 44, 598-602). In particular embodiments, the threshold level is 300 μmol/L or less, for example, 250 μmol/L or less, 200 μmol/L or less, 150 μmol/L or less, 100 μmol/L or less, 75 μmol/L or less, 50 μmol/L or less, 30 μmol/L or less. In some embodiments, the threshold level is 13 nM. In some embodiments, the threshold level is 37 nM. In some embodiments, saliva samples are taken at least 2 hours after the subject has had the last meal, to eliminate the spike in bile acid levels that shortly follows a meal. But in some embodiments, saliva samples are taken within 2 hours after the subject has had the last meal. Subjects thus identified to have an elevated level of total bile acids are then administered with the gastric-retentive bile acid sequestrant composition as described elsewhere. To minimize variability, it is best to collect the saliva samples in as consistent a manner as possible, taking into account the time since the last dose of gastric-retentive bile acid sequestrant composition, the time since last dose of PPI, time of day, etc.

In yet another aspect, a method of titrating an optimal dose of a gastric-retentive bile acid sequestrant composition is described. The method comprises administering a first dose of a gastric-retentive bile acid sequestrant composition, then obtaining a saliva sample from the subject. The subject is then provided with a second dose, and a second saliva sample is obtained. The subject can optionally be administered a third dose, after which a third saliva sample is obtained. The total bile acid levels of the saliva samples are determined using the method described herein. The lowest dose that yields saliva total bile acid levels below a threshold level is deemed to be the optimal dose. In certain embodiments, the threshold level is 300 μmol/L or less, for example, 250 μmol/L or less, 200 μmol/L or less, 150 μmol/L or less, 100 μmol/L or less, 75 μmol/L or less, 50 μmol/L or less, 30 μmol/L or less.

The saliva may be collected from a subject by any suitable method known in the art. The bile acid levels may also be determined by any suitable method known in the art, such as, for example and without limitation, liquid chromatography with tandem mass spectrometry (LC-MS/MS).

This disclosure provides a method for detecting and quantifying bile acids from saliva from a human patient, comprising: collecting saliva and determining the bile acid levels in the saliva using liquid chromatography with tandem mass spectrometry. The level of individual bile acid can be determined by correlating the levels determined for that bile acid in a standard curve.

In certain embodiments, the human patient is being treated with a gastro-retentive oral dosage form comprising a bile acid sequestrant. In other embodiments, the human patient is being treated with another suitable active agent.

In certain embodiments, the human patient is being treated with an enteric coated gastro-retentive oral dosage form in the form of a tablet of a bile acid sequestrant. In certain embodiments, the bile acid sequestrant is dispersed in a polymeric matrix. In certain embodiments, the polymeric matrix consist essentially of poly(alkylene)oxide. In certain embodiments, the gastro-retentive oral dosage form comprises one or more filler or compressing agent selected from microcrystalline cellulose, butylated hydroxytoluene, colloidal silicon dioxide, lactose, starch, maltodextrins, magnesium stearate, diacetylated monoglycerides, hypromellose, and dibasic calcium phosphate. In certain embodiments, the tablet has a tablet core and is coated with an enteric coating, which in certain further embodiments is a polyvinyl alcohol based enteric coating (such as Opadry® II 85F), for prolonged retention of the bile acid sequestrant in the stomach of the patient. In certain embodiments, the human patient is also administered a pharmaceutical composition comprising a PPI. In some embodiments, the patient experiences a clinically meaningful reduction in one or more symptoms of GERD.

Other oral dosage forms comprising a bile acid sequestrant are disclosed in U.S. Pat. No. 9,205,094 and WO2014/113377.

Bile acid sequestrants include, for example, cholestyramine (i.e., QUESTRAN®, QUESTRAN LIGHT®, CHOLYBAR®, CA registry no. 11041-12-6), colesevelam (i.e., WELCHOL®, CA registry nos. 182815-43-6 and 182815-44-7), Selevamer (Rinogel®) and colestipol (i.e., COLESTID®, CA registry nos. 50925-79-6 and 37296-80-3), or any of their pharmaceutically acceptable salts or mixtures thereof. Colesevelam or colesvelam HCl (may be referred to herein jointly as colesevelam) is an orally administered, nonabsorbed, nondigestible polymer that binds bile acids in the GI tract. Colesevelam was approved in 2000 in the United States (US) as the active ingredient in Welchol™ and is indicated as an adjunct to diet and exercise for reduction of elevated low-density lipoprotein cholesterol (LDL-C) in adults with primary hyperlipidemia. Colesevelam is currently available as an immediate-release formulation only. Colesevelam is not systemically absorbed and does not interfere with systemic drug metabolizing enzymes. Distribution of colesevelam is limited to the GI tract and elimination occurs through fecal excretion.

In certain embodiments, the bile acid sequestrant is administered to a patient at 500 mg, 700 mg, 750 mg, 1,000 mg, 1,400 mg, 1,500 mg, 2,000 mg, 2,100 mg, or more. In some embodiments, the bile acid sequestrant is administered to a patient, one dose per day, two dose per day, or 3 dose per day. In certain embodiments, the bile acid sequestrant is administered to a patient as three 500 mg tablets twice per day.

In other embodiments, the human patient has symptomatic GERD not completely responsive to proton pump inhibitors (PPI), and is being treating by a therapeutically effective amount of an enteric coated gastro-retentive oral dosage form in the form of a tablet of colesevelam or colesevelam hydrochloride a dispersed in a polymeric matrix comprising of or consisting essentially of polyethylene oxide CAS Number 25322-68-3, approximate molecular weight 300,000 (PEG-7M) and, in certain further embodiments, one or more filler or compressing agent selected from microcrystalline cellulose, lactose, starch, maltodextrins and dibasic calcium phosphate, wherein, in certain embodiments, the tablet is coated with a polyvinyl alcohol based enteric coating, for prolonged retention of the bile acid sequestrant in the stomach of the patient in a dose of 1,500 mg twice daily; wherein: prior to administering said enteric coated gastro-retentive, oral dosage form in the form of a tablet of a bile acid sequestrant, the patient was not completely responsive to other treatments, including, in some embodiments, individually optimized, standard-labeled dose daily PPI therapy for a minimum of 8 weeks. In some embodiments, the patient has erosive esophagitis. In some embodiments, the patient is treated by said enteric coated gastro-retentive, oral dosage form in the form of a tablet of a bile acid sequestrant for eight weeks (eight treatment weeks) or more. In some embodiments, the dosage form is retained in the stomach until it is substantially or completely disintegrated.

In still other embodiments, the human patient is administered the enteric coated gastro-retentive oral dosage form in the form of a tablet of a bile acid sequestrant in an amount effective to reduce the saliva bile acid levels by at least 10%, when measured more than 2 hours after a meal. In other embodiments, the subject is administered with the composition in an amount effective to reduce the saliva total bile acid levels by at least 15%, for example, at least 20%, at least 30%, at least 40%, at least 50% or more, when compared with levels prior to administration of said composition when measured more than 2 hours after a meal.

In still other embodiments, the human patient is administered an enteric coated gastro-retentive oral dosage form in the form of a tablet of a bile acid sequestrant in an amount effective to reduce the saliva bile acid levels to 200 μmol/L or below when measured more than 2 hours after a meal. In other embodiments, the subject is administered with the composition in an amount effective to reduce the saliva total bile acid levels to 150 μmol/L or below, 100 μmol/L or below, 75 μmol/L or below, 50 μmol/L or below, or lower, when measured more than 2 hours after a meal.

In certain embodiments, the bile acid sequestrant is colesevelam or colesevelam hydrochloride.

In certain embodiments, each dose of the enteric coated gastro-retentive oral dosage form in the form of a tablet for prolonged retention of the bile acid sequestrant in the stomach of the patient is in a dose of 500 mg, 700 mg, 750 mg, 1,000 mg, 1,400 mg, 1,500 mg, 2,000 mg, 2,100 mg, or more. In certain further embodiments, the dose is administered twice per day.

A dose may be several dosage forms (tablets) disclosed herein or only one. In certain embodiments, two tablets are administered to the patient twice per day. In other embodiments, three tablets are administered to the patient twice per day.

In certain embodiments, an ingredient of this polymeric matrix is at least one hydrophilic, water-swellable, erodible, or soluble polymer, which may generally be described as an “osmopolymer”, “hydrogel” or “water-swellable” polymer. More than one of such polymers may be combined in a dosage form of the invention to achieve gastric-retention as well as the desired erosion rate.

Polymers suitable for achieving the desired gastro-retentive and sustained-release profiles of the dosage forms used in the methods disclosed herein have the property of swelling as a result of imbibing water from the gastric fluid, and gradually eroding over a time period of several hours. Since erosion of the polymer results from the interaction of the fluid with the surface of the dosage form, erosion initiates more or less simultaneously with the swelling process. While erosion and swelling may occur at the same time, the rate for achieving maximum swelling should be faster than the rate the dosage form fully erodes to achieve the desired release profile. Such polymers may be linear, branched, or cross linked. The polymers may be homopolymers or copolymers.

In some embodiments, the polymer is a polyalkylene oxide. In some embodiments, at least one of the one or more hydrophilic polymers is a polyethylene oxide (PEO). In still other embodiments, the at least one hydrophilic polymer is a polyethylene oxide having a molecular weight of about 300,000 Daltons.

Polyethylene oxide (PEO) is a polyethylene oxide polymer that has a wide range of molecular weights. PEO is a linear polymer of unsubstituted ethylene oxide and has a wide range of viscosity-average molecular weights. Non-limiting examples of commercially available PEOs and their approximate molecular weights (in grams/mole or Daltons) are: POLYOX® NF, grade WSR coagulant, approximate molecular weight 5 million; POLYOX® grade WSR 301, approximate molecular weight 4 million; POLYOX® grade WSR 303, approximate molecular weight 7 million; POLYOX® grade WSR N60-K, approximate molecular weight 2 million; POLYOX® grade WSR N-80K, approximate molecular weight 200,000; Polyox™ WSR N-750 (INCI name: PEG-7M), which is a polymer of ethylene oxide, approximate molecular weight 300,000 (polyethylene oxide CAS Number 25322-68-3, approximate molecular weight 300,000 (PEG-7M)).

In some embodiments, the polyethylene oxide is present in the unit dosage form in an amount ranging from 40 weight percent ratio to 75 weight percent ratio. In some embodiments, the polyethylene oxide is present in the unit dosage form in an amount ranging from 40 weight percent ratio to 60 weight percent ratio. In some embodiments, the polyethylene oxide is present in the unit dosage form in an amount ranging from 45 weight percent ratio to 55 weight percent ratio. In some embodiments, the poly(ethylene)oxide is present in the unit dosage form in an amount ranging from 45 weight percent ratio to 60 weight percent ratio. In some embodiments, the polyethylene oxide is present in the unit dosage form in an amount ranging from 40 weight percent ratio to 50 weight percent ratio. In some embodiments, the poly(ethylene)oxide is present in the unit dosage form in an amount ranging from 50 weight percent ratio to 60 weight percent ratio. In some embodiments, the poly(ethylene)oxide is present in the unit dosage form in an amount ranging from 47 weight percent ratio to 53 weight percent ratio.

In certain embodiments, the poly(alkylene)oxide is polyethylene oxide CAS Number 25322-68-3, approximate molecular weight 300,000 (PEG-7M) (Polyox™ WSR N-750). The term “PEG-7M” herein refers to polyethylene oxide CAS Number 25322-68-3, approximate molecular weight 300,000 (PEG-7M) (Polyox™ WSR N-750). In certain embodiments, the polyalkylene oxide is polyethylene oxide CAS Number 25322-68-3, approximate molecular weight 300,000 (PEG-7M) (Polyox™ WSR N-750) at about 30% to about 46% to about 60% w/w of the tablet core weight. The tablets have a core, which in turn is coated to become a coated tablet. In certain embodiments, the poly(alkylene) oxide has approximate molecular weight of 300,000 Daltons. In certain embodiments, the poly(alkylene)oxide yields viscosity of 600 to 1,000 at moderate addition levels.

In other embodiments, the at least one hydrophilic polymers of the dosage form include a cellulose. In certain embodiments, the polymers may be synthetic polymers derived from vinyl, acrylate, methacrylate, urethane, ester and oxide monomers. In other embodiments, they can be derivatives of naturally occurring polymers such as polysaccharides (e.g. chitin, chitosan, dextran and pullulan; gum agar, gum arabic, gum karaya, locust bean gum, gum tragacanth, carrageenans, gum ghatti, guar gum, xanthan gum and scleroglucan), starches (e.g. dextrin and maltodextrin, corn-starch-unmodified or pregelatinized-), hydrophilic colloids (e.g. pectin), phosphatides (e.g. lecithin), alginates (e g ammonium alginate, sodium, potassium or calcium alginate, propylene glycol alginate), gelatin, collagen, and cellulosics. Cellulosics are cellulose polymer that has been modified by reaction of at least a portion of the hydroxyl groups on the saccharide repeat units with a compound to form an ester-linked or an ether-linked substituent. For example, the cellulosic ethyl cellulose has an ether linked ethyl substituent attached to the saccharide repeat unit, while the cellulosic cellulose acetate has an ester linked acetate substituent.

In certain embodiments, the cellulosics for the erodible matrix comprises aqueous-soluble and aqueous-erodible cellulosics can include, for example, methylethyl cellulose (MEC), carboxymethyl cellulose (CMC), CMEC, hydroxyethyl cellulose (HEC), hydroxypropyl cellulose (HPC), cellulose acetate (CA), cellulose propionate (CP), cellulose butyrate (CB), cellulose acetate butyrate (CAB), CAP, CAT, hydroxypropyl methyl cellulose (HPMC), HPMCP, HPMCAS, hydroxypropyl methyl cellulose acetate trimellitate (HPMCAT), and ethylhydroxy ethylcellulose (EHEC). In certain embodiments, the cellulosics comprises various grades of low viscosity (MW less than or equal to 50,000 Daltons, for example, the Dow Methocel™ series E5, E15LV, E50LV and K100LY) and high viscosity (MW greater than 50,000 Daltons, for example, E4MCR, E10MCR, K4M, K15M and K100M and the Methocel™ K series) HPMC. Other commercially available types of HPMC include the Shin Etsu Metolose 90SH series.

Other materials useful as the erodible matrix material include, but are not limited to, pullulan, polyvinyl pyrrolidone (povidone), polyvinyl alcohol, polyvinyl acetate, glycerol fatty acid esters, polyacrylamide, polyacrylic acid, copolymers of ethacrylic acid or methacrylic acid (EUDRAGIT®, Rohm America, Inc., Piscataway, N.J.) and other acrylic acid derivatives such as homopolymers and copolymers of butylmethacrylate, methylmethacrylate, ethylmethacrylate, ethylacrylate, (2-dimethylaminoethyl) methacrylate, and (trimethylaminoethyl) methacrylate chloride.

In some embodiments, the hydrophilic polymer is used as a binder in the unit dosage form and is selected from povidone, starch, hydroxypropylcellulose, and hydroxypropylmethylcellulose.

In some embodiments, the tablets used in the methods disclosed herein comprise a core and an enteric coating. The enteric coating surrounding the core may be applied using standard coating techniques. Materials used to form the enteric coating may be dissolved or dispersed in organic or aqueous solvents and may include one or more of the following: methacrylic acid copolymers; shellac; hydroxypropylmethylcellulose phthalate; polyvinyl acetate phthalate; hydroxypropylmethylcellulose trimellitate; carboxymethylcellulose; cellulose acetate phthalate; or other suitable enteric coating polymers. The pH at which the enteric coat will dissolve can be controlled by the polymer or combination of polymers selected and/or ratio of pendant groups. For example, dissolution characteristics of the coating can be altered by the ratio of free carboxyl groups to ester groups. Enteric coating layers may also contain pharmaceutical plasticizers such as: triethyl citrate; dibutyl phthalate; triacetin; polyethylene glycols; polysorbates; acetylated glycerides, etc. Additives such as dispersants, colorants, anti-adhering, taste-masking and anti-foaming agents may also be included. Any suitable enteric coating may be used. In certain embodiments, the enteric coating is a polyvinyl alcohol (PVA)-based coating composition such as Opadry® II 85 supplied by Colorcon. Opadry Enteric is a platform of fully formulated delayed release coating systems from Colorcon.

In some embodiments, the gastro-retentive dosage forms can be prepared by any suitable process. Methods of making the dosage forms and tablets used in the methods disclosed herein are known. See U.S. Pat. No. 9,205,094 and WO2014/113377.

In certain embodiments, prior to this treatment, the patient has not been completely responsive to other treatments, including individually optimized, standard-labeled dose daily PPI therapy for a minimum of 8 weeks prior to this treatment.

The human patients may have a disease selected from heartburn, indigestion, dyspepsia, erosive esophagitis, peptic ulcer, gastric ulcer, esophageal ulcers, esophagitis, laryngitis, pharyngitis, coarse voice, gastroesophageal reflux disease (GERD), Barrett's esophagus, gastric cancer, esophageal cancer (e.g., adenocarcinoma), and gastritis and GERD-related pulmonary dysfunction, instead of, or in addition to, patients with symptomatic GERD not completely responsive to proton pump inhibitor.

In certain embodiments, an enteric coated oral dosage form described herein further comprises butylated hydroxytoluene (BHT). In certain embodiments, the disclosed oral dosage form comprises about 0.01 mg to about 1.5 mg of BHT. In certain embodiments, the disclosed oral dosage form comprises at least about 0.06% BHT by weight per tablet core; the 0.06% BHT are added to the formulation.

These dosage forms, formulations and pharmaceutical compositions are formulated with suitable carriers, excipients, and other agents that provide suitable transfer, delivery, tolerance, and the like. A multitude of appropriate formulations can be found in the formulary known to all pharmaceutical chemists: Remington's Pharmaceutical Sciences, Mack Publishing Company, Easton, Pa. These formulations include, for example, powders, pastes, ointments, jellies, waxes, oils, lipids, lipid (cationic or anionic) containing vesicles (such as LIPOFECTIN™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol 52:238-311.

The enteric coated gastro-retentive, oral dosage forms in the form of a tablet are intended for oral delivery to a patient.

In certain embodiments, the dosage form may additionally contain suitable diluents, glidants, lubricants, acidulants, stabilizers, fillers, binders, plasticizers or release aids and other pharmaceutically acceptable excipients.

In certain embodiments, the dosage form comprises one or more of microcrystalline cellulose (at between 1-10% w/w of the tablet core), butylated hydroxytoluene oxide (at between 0.01-0.10% w/w of the tablet core), colloidal silicon oxide (at between 1.0-2.5% w/w of the tablet core) and magnesium stearate (at between 0.1-1.0% w/w of the tablet core).

In certain embodiments, the enteric coating is a polyvinyl alcohol (PVA)-based coating composition, such as Opadry® II 85 supplied by Colorcon. Opadry Enteric is a platform of fully formulated delayed release coating systems from Colorcon. In certain embodiments, the tablets are coated with 1-4% Opadry® II 85F w/w of the coated tablet.

In certain embodiments, the one or more filler or compressing agent of the oral dosage form comprising a bile acid sequestrant is microcrystalline cellulose at 1-10% w/w of the tablet, butylated hydroxytoluene at 0.01 to 0.10% w/w of the tablet, colloidal silicon dioxide at 1-5% w/w of the tablet, and/or magnesium stearate at 0.1 to 1.0% w/w of the tablet. In certain embodiments, the one or more filler or compressing agent is microcrystalline cellulose at 5.4% w/w of the tablet, butylated hydroxytoluene at 0.06 w/w of the tablet, colloidal silicon dioxide at 2.0% w/w of the tablet, and/or magnesium stearate at 0.5% w/w of the tablet.

In certain embodiments, the enteric coating of the oral dosage form comprising a bile acid sequestrant is a polyvinyl alcohol based enteric coating. In certain embodiments, the enteric coating of the oral dosage form comprising a bile acid sequestrantis a polyvinyl alcohol based enteric coating is Opadry II 85F. In certain embodiments, the enteric coating of the oral dosage form comprising a bile acid sequestrant is a polyvinyl alcohol based enteric coating is Opadry II 85F at 1-5% w/w of the tablet. In further embodiments, the enteric coating is a polyvinyl alcohol based enteric coating is Opadry II 85F at 3% w/w of the tablet.

In certain embodiments, the PEG-7M (Polyox™ WSR N-750) is at about 30 to about 60% w/w of the tablet. In further embodiments, the PEG-7M (Polyox™ WSR N-750) is at about 46% w/w of the tablet.

The methods disclosed herein may be used to treat patients using combination therapy, comprising administering a gastric-retentive oral dosage forms comprising at least one bile acid sequestrant in combination with one or more additional therapeutic agents. For combination treatment with more than one active agent, where the active agents may be in separate dosage forms, the active agents may be administered separately or in conjunction. In addition, the administration of one agent may be prior to, concurrent to, or subsequent to the administration of the other agent.

As used herein, the terms “in combination” or “co-administration” can be used interchangeably to refer to the use of more than one therapy (e.g., one or more prophylactic and/or therapeutic agents). The use of the terms does not restrict the order in which therapies (e.g., prophylactic and/or therapeutic agents) are administered to a subject.

In some embodiments, the methods further comprise administering to the patient simultaneously, separately, or sequentially, one or more proton pump inhibitors (PPI). In certain embodiments, the PPI is administered QD (once-per-day).

In other embodiments, the methods further comprise administering simultaneously, separately or sequentially, one or more acid pump antagonists.

In other embodiments, the methods further comprise administering simultaneously, separately, or sequentially one or more agents chosen from an antacid, a histamine H2-receptor antagonist, a γ-aminobutyric acid-β (GABA-B) agonist, a prodrug of a GABA-B agonist, and a protease inhibitor.

While the two or more agents in the combination therapy can be administered simultaneously, they need not be. For example, administration of a first agent (or combination of agents) can precede administration of a second agent (or combination of agents) by minutes, hours, days, or weeks.

Combination therapy can also include two or more administrations of one or more of the agents used in the combination.

PPI drugs are substituted benzimidazole compounds that specifically inhibit gastric acid secretion by affecting the H⁺/K⁺ ATPase enzyme system (the proton pump). These drugs, for example esomeprazole, are rapidly absorbed and have very short half-lives. However, they exhibit prolonged binding to the H⁺/K⁺ ATPase enzyme. The anti-secretory effect reaches a maximum in about 4 days with once-daily dosing. Because of these characteristics, patients beginning PPI therapy do not receive maximum benefit of the drug and healing may not begin for up to 5 days after therapy begins when PPIs are used alone for initial therapy of upper GI tract disorders.

Proton pump inhibitors (PPIs) are potent inhibitors of gastric acid secretion, inhibiting H⁺/K⁺ ATPase, the enzyme involved in the final step of hydrogen ion production in the parietal cells. The term proton pump inhibitor includes, but is not limited to, omeprazole (as sold under the brand-names PRILOSEC®, LOSEC, or ZEGERID®), lansoprazole (as sold under the brand-name PREVACID®, ZOTON®, or INHIBITOL®), rabeprazole (as sold under the brand-name RABECID®, ACIPHEX®, or PARIET®), pantoprazole (as sold under the brand-name PROTONIX®, PROTIUM®, SOMAC®, or PANTOLOC®), tenatoprazole (also referred to as benatoprazole), and leminoprazole, including isomers, enantiomers and tautomers thereof (e.g., esomeprazole (as sold under the brand-name NEXIUM®)), Dexlansoprazole, Dexrabeprazole, (S)-Pantoprazole, Ilaprazole and alkaline salts thereof; The following patents describe various benzimidazole compounds suitable for use in the disclosure described herein: U.S. Pat. Nos. 4,045,563, 4,255,431, 4,359,465, 4,472,409, 4,508,905, JP-A-59181277, U.S. Pat. Nos. 4,628,098, 4,738,975, 5,045,321, 4,786,505, 4,853,230, 5,045,552, EP-A-295603, U.S. Pat. No. 5,312,824, EP-A-166287, U.S. Pat. No. 5,877,192, EP-A-519365, EP5129, EP 174,726, EP 166,287 and GB 2,163,747. Thus, proton pump inhibitors and their pharmaceutically acceptable salts, which are used in accordance with the present disclosure, are known compounds and can be produced by known processes. In certain embodiments, the proton pump inhibitor is omeprazole, either in racemic mixture or only the (−) enantiomer of omeprazole (i.e. esomeprazole), as set forth in U.S. Pat. No. 5,877,192, hereby incorporated by reference.

Omeprazole is typically administered in a 20 mg dose/day for active duodenal ulcer for 4-8 weeks; in a 20 mg dose/day for gastro-esophageal reflux disease (GERD) or severe erosive esophagitis for 4-8 weeks; in a 20 mg dose/twice a day for treatment of Helicobacter pylori (in combination with other agents); in a 60 mg dose/day for active duodenal ulcer for 4-8 weeks and up to 120 mg three times/day, and in a 40 mg dose/day for gastric ulcer for 4-8 weeks. In other embodiments of the present disclosure, the dose of proton pump inhibitor is sub-therapeutic.

Lansoprazole is typically administered about 15-30 mg/day; rabeprazole is typically administered 20 mg/day and pantoprazole is typically administered 40 mg/day. However, any therapeutic or sub-therapeutic dose of these agents is considered within the scope of the present disclosure.

Acid pump antagonists (APAs) acting by K(+)-competitive and reversible (as opposed to irreversible PPIs) binding to the gastric proton pump, which is the final step for activation of acid secretion in the parietal cell. One class of APAs are imidazopyridines. BY841 was selected from this class and is chemically a (8-(2-methoxycarbonylamino-6-methyl-phenylmethylamino)-2,3-dimethyl-imidazo [1,2-a]-pyridine). In pharmacological experiments such as pH-metry in the conscious, pentagastrin-stimulated fistula dog, BY841 proved to be superior to both ranitidine and omeprazole by rapidly elevating intragastric pH up to a value of 6. The duration of this pH elevation in the dog was dose-dependent. Using both acid output and continuous 24-hr pH measurements, a pronounced antisecretory effect of BY841 has been found. Actually, a single 50 mg oral dose of BY841 immediately elevated intragastric pH to about 6. Higher doses caused a dose-dependent increase in duration of the pH-elevation, without any further increase in maximum pH values. Twice daily administration was more effective than once a day administration of the same daily dose. With both regimens, the duration of the pH-elevating effect of BY841 further increased upon repeated daily administration. This demonstrates lack of tolerance development, the latter being a well-known disadvantage of H2-receptor antagonists. In comparison with the standard dose of omeprazole, BY841 administered at a dose of 50 mg or 100 mg twice daily is markedly more effective on Day one of treatment, and both doses are at least as potent as omeprazole following repeated daily administration.

Examples of some APAs include, but are not limited to: BY-841 (Prumaprazole), Sch-28080, YJA-20379-8, YJA-20379-1, SPI-447, SK&F-97574, AU-2064, SK&F-96356, T-330, SK&F-96067, SB-641257A (YH-1885, Revaprazan hydrochloride, RevanexR), CS-526, R-105266, Linaprazan, Sorapraza, DBM-819, KR-60436, RQ-00000004 (RQ-4) and YH-4808.

Other agents include: histamine H2 receptor blockers, motility agents (gastroprokinetics), antacids, antiulcerative agents, γ-aminobutyric acid-β (GABA-B) agonists, prodrugs of GABA-B agonists, GCC agonists and/or protease inhibitors. Non-limiting examples of these additional agents include: cinitapride, cisapride, fedotozine, loxiglumide, alexitol sodium, almagate, aluminum hydroxide, aluminum magnesium silicate, aluminum phosphate, azulene, basic aluminum carbonate gel, bismuth aluminate, bismuth phosphate, bismuth subgallate, bismuth subnitrate, calcium carbonate, dihydroxyaluminum aminoacetate, dihydroxyaluminum sodium carbonate, ebimar, magaldrate, magnesium carbonate hydroxide, magnesium hydroxide, magnesium oxide, magnesium peroxide, magnesium phosphate (tribasic), magnesium silicates, potassium citrate, sodium bicarbonate, aceglutamide aluminum complex, acetoxolone, aldioxa, arbaprostil, benexate hydrochloride, carbenoxolone, cetraxate, cimetidine, colloidal bismuth subcitrate, ebrotidine, ecabet, enprostil, esaprazole, famotidine, gefamate, guaiazulene, irsogladine, misoprostol, nizatidine, omoprostil, -Oryzanol, pifamine, pirenzepine, plaunotol, polaprezinc, ranitidine, rebamipide, rioprostil, rosaprostol, rotraxate, roxatidine acetate, sofalcone, spizofarone, sucralfate, telenzepine, teprenone, trimoprostil, trithiozine, troxipide, zolimidine, baclofen, R-baclofen, XP19986 (CAS Registry No. 847353-30-4), pepstatin and other pepsin inhibitors (e.g., sodium benzoate); and chymotrypsin and trypsin inhibitors. A wide variety of trypsin and chymotrypsin inhibitors are known to those skilled in the art and can be used in the methods described herein. Such trypsin and chymotrypsin inhibitors can include tissue-factor-pathway inhibitor; α-2 antiplasmin; serpin α-1 antichymotrypsin family members; gelin; hirustasin; eglins including eglin C; inhibitors from Bombyx mori (see; e.g.; JP 4013698 A2 and JP 04013697 A2; CA registry No. 142628-93-1); hirudin and variants thereof; secretory leukocyte protease inhibitor (SLPI); α-1 anti-trypsin; Bowman-Birk protease inhibitors (BBIs); chymotrypsin inhibitors represented by CAS registry Nos. 306762-66-3, 306762-67-4, 306762-68-5, 306762-69-6, 306762-70-9, 306762-71-0, 306762-72-1, 306762-73-2, 306762-74-3, 306762-75-4, 178330-92-2, 178330-93-3, 178330-94-4, 81459-62-3, 81459-79-2, 81460-01-7, 85476-59-1, 85476-62-6, 85476-63-7, 85476-67-1, 85476-70-6, 85858-66-8, 85858-68-0, 85858-69-1, 85858-70-4, 85858-71-5, 85858-72-6, 85858-73-7, 85858-75-9, 85858-77-1, 85858-79-3, 85858-81-7, 85858-83-9, 85858-84-0, 85858-85-1, 85858-87-3, 85858-89-5, 85858-90-8, 85858-92-0, 85879-03-4, 85879-05-6, 85879-06-7, 85879-08-9, 85858-74-8, 90186-24-6, 90185-93-6, 89703-10-6, 138320-33-9 (Y53025), 94149-41-4 (MR889), 85858-76-0, 89703-10-6, 90185-92-5, 90185-96-9, 90185-98-1, 90186-00-8, 90186-01-9, 90186-05-3, 90186-06-4, 90186-07-5, 90186-08-6, 90186-09-7, 90186-10-0, 90186-11-1, 90186-12-2, 90186-13-3, 90186-14-4, 90186-22-4, 90186-23-5, 90186-24-6, 90186-25-7, 90186-27-9, 90186-28-0, 90186-29-1, 90186-31-5, 90186-35-9, 90186-43-9, 90209-88-4, 90209-89-5, 90209-92-0, 90209-94-2, 90209-96-4, 90209-97-5, 90210-01-8, 90210-03-0, 90210-04-1, 90210-25-6, 90210-26-7, 90210-28-9, 90230-84-5, 90409-84-0, 95460-86-9, 95460-87-0, 95460-88-1, 95460-89-2, 95460-91-6, 114949-00-7, 114949-01-8, 114949-02-9, 114949-03-0, 114949-04-1, 114949-05-2, 114949-06-3, 114949-18-7, 114949-19-8, 114964-69-1, 114964-70-4, 9076-44-2 (chymostatin), 30827-99-7 (Pefabloc), 618-39-3 (benzamidine), 80449-31-6 (urinistatin), 130982-43-3, 197913-52-3, 179324-22-2, 274901-16-5, 792163-40-7, 339169-59-4, 243462-36-4, 654671-78-0, 55123-66-5 (leupeptin), 901-47-3, 4272-74-6, 51050-59-0, 221051-66-7, 80449-31-6, 55-91-4, 60-32-2, 88070-98-8, 87928-05-0, 402-71-1 (benzenesulfonamide), 139466-47-0, CI-2A (see U.S. Pat. No. 5,167,483), CI-2A (seebWO9205239), WCI-3 (see Shibata et al. 1988 J Biochem (Tokyo) 104:537-43), WCI-2 (see Habu et al. 1992 J Biochem (Tokyo) 111:249-58), and WCI-x (Habu et al., supra) and 178330-95-5; and compounds with chymotrypsin inhibition activity described in patent publications JP 56092217 A2, U.S. Pat. Nos. 4,755,383, 4,755,383, 4,639,435, 4,620,005, 4,898,876, and EP0128007.

Examples of other therapeutic agents that may be combined with a compound of this disclosure, either administered separately or in the same pharmaceutical composition, include, but are not limited to linaclotide, IW-9179, plecanatide and SP-333.

Any additional suitable agents may be administered to the patient.

A method of monitoring progress of GERD, wherein samples of a subject who is being given a bile acid lowering or sequestering agent is monitored, and a reduction in bile acid levels is indicative of effective therapy.

### EXAMPLES

For this invention to be better understood, the following examples are set forth. These examples are for purposes of illustration only and are not be construed as limiting the scope of the invention in any manner.

### Example 1: Development of a Quantitative Method for the Detection of Bile Acids from Human Saliva

Introduction: Current methods for detecting bile acids from subjects suffer from major drawbacks. For example, the Bilitec® ambulatory bile reflux monitor actually detects bilirubin as a surrogate of bile acids (Barrett et al., (2000) Dis. Esophagus, 13, 44-50), and thus cannot be used to quantitate the levels of different bile acids. The Bilitec® assay is a disruptive procedure requiring placement a tube through the nose into the esophagus; cannot be used to detect low levels of bile acids; and has only a marginal correlation to bile acid levels (Barrett et al., id.). There is, therefore, a need for a non-invasive method for testing for the presence and quantification of individual bile acid levels.

Method: In general terms, the method disclosed herein is a simple, quantitative and non-invasive method for the detection of bile acids from fluids, including saliva. The experiments summarized below makes use of saliva samples as little as 50 μl in volume, but can be adapted to accommodate even lower volumes. Saliva samples are processed and subjected to LC/MS/MS analysis, and compared with control samples containing internal samples.

Collection of saliva: saliva is readily collected from a subject using a collection device, for example, the SalivaBio Oral Swab (Salimetrics, Carlsbad Calif.), etc.

Preparation of Samples:

50 μl of saliva is removed from the collection tube and mixed with 350 μl of ice cold acetonitrile (CAN).

Samples are centrifuged at 3200 rpm for 5 min at 4° C. The supernatant is transferred to a new tube and lyophilized. The dried sample is reconstituted in 50 μl of a solution of 50% (v/v) methanol.

TABLE 1
LC/MS/MS Conditions

| | |
|---|---|
| HPLC: | Waters Aquity |
| Column: | Thermo Hypersil Gold C18 50 × 2.1, 1.9 um |
| Guard Column: | Hypersil Gold C₁₈ |
| Column Temperature: | RT (~23° C.) |
| Injection Volume: | 10 μL |
| Autosampler Temperature: | 4° C. |
| Flow Rate: | 0.5 mL/min |
| Mobile Phases: | A: Water with 0.2% Formic Acid |
| | B: Acetronitrile with 0.2% Formic Acid |

| Gradient | Time (min) | % A | % B |
|---|---|---|---|
| | 0 | 70 | 30 |
| | 0.3 | 70 | 30 |
| | 2.6 | 62 | 38 |
| | 6.0 | 2 | 98 |
| | 7.0 | 2 | 98 |
| | 7.1 | 70 | 30 |

| | |
|---|---|
| 8 | (end) |

| | Detection |
|---|---|
| Mass Spectrometer | Sciex 5500 |
| Ion Mode | Negative |
| Source Temperature | 150° C. |

Internal Standards

LC experiments using saliva samples to which an individual species of bile acid was ‘spiked’ were performed to determine the retention time on the column.

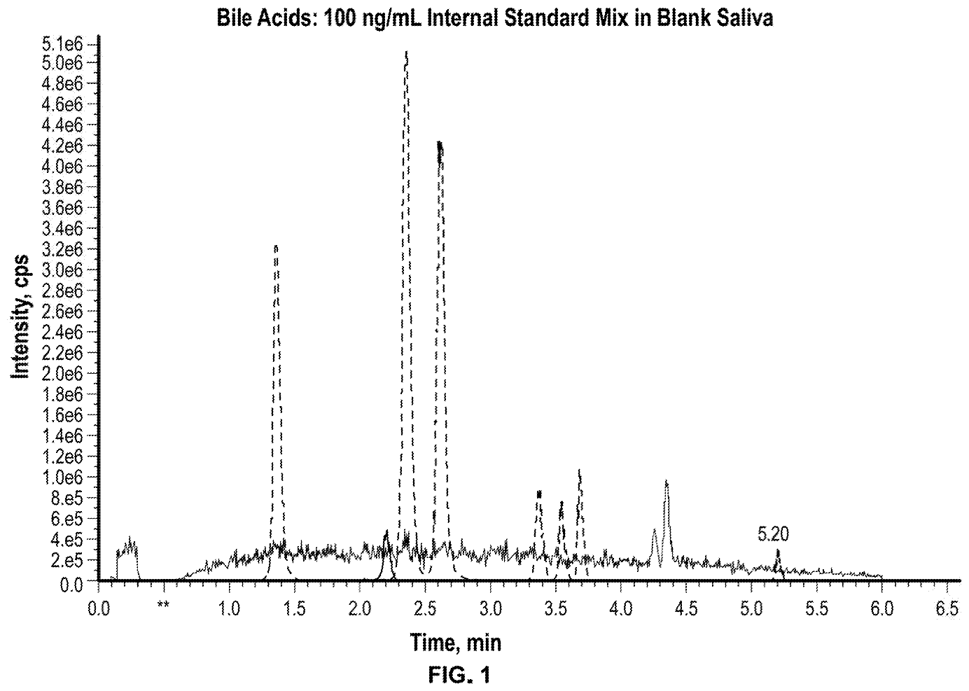

FIG. 1 shows the LC/MS/MS profile of an internal standard of ‘spiked’ bile acids provided in Table 2.

TABLE 2

| | Mass | Dwell Time | DP | CE | CXP |
|---|---|---|---|---|---|
| Compound | Transition | (msec) | (V) | (V) | (V) |
| Glycocholic acid (GCA) | 464.2 > 74 | 50 | −120 | −77 | −1 |
| Deoxycholic acid (DCA) | 391 > 391 | 50 | −130 | −20 | −7 |
| Chenodeoxycholic acid (CDCA) | 391 > 391 | 50 | −130 | −20 | −7 |
| Glycochenodeoxycholic acid (GCDCA) | 448.2 > 74 | 50 | −115 | −70 | −1 |
| Glycodeoxycholic acid (GDCA) | 448.2 > 74 | 50 | −115 | −70 | −1 |
| Cholic Acid (CA) | 407.2 > 407.2 | 50 | −130 | −30 | −14 |
| Taurocholoc acid (TCA) | 514.2 > 79.8 | 50 | −165 | −125 | −1 |
| Lithocholic acid (LCA) | 375.2 > 375.2 | 50 | −130 | −30 | −7 |
| Taurodeoxycholic acid (TDCA) | 498.2 > 79.8 | 50 | −165 | −125 | −1 |
| Taurochenodeoxycholic acid (TCDCA) | 498.2 > 79.8 | 50 | −165 | −125 | −1 |
| D4 GCA | 468.3 > 74 | 50 | −120 | −72 | −1 |

Standard Curve

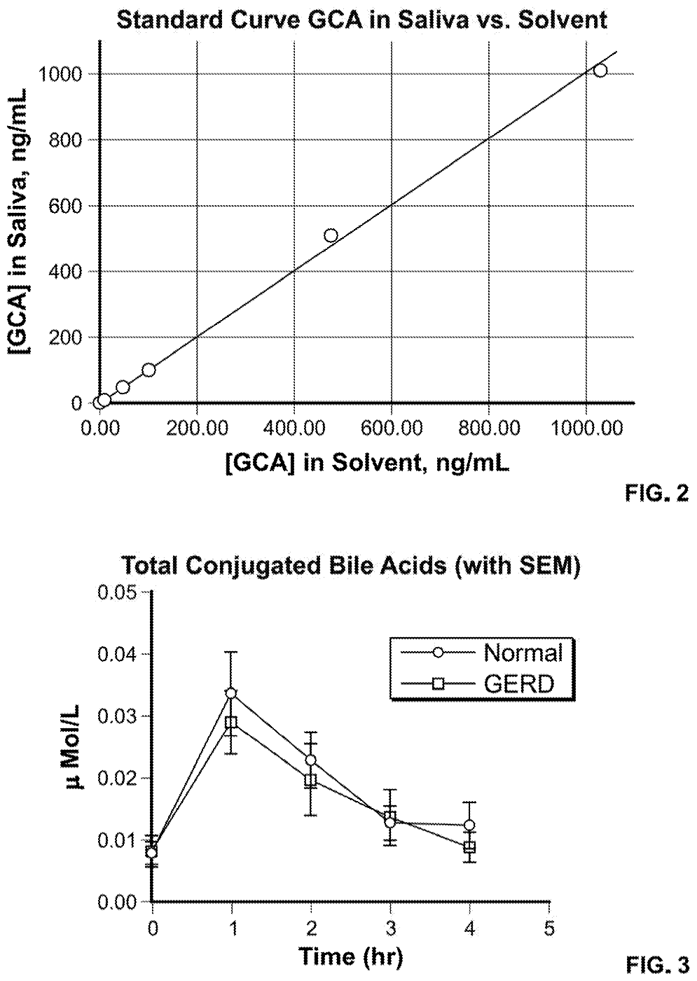

In this experiment, Glycocholic acid (GCA) was used to develop a standard curve. GCA standards of 0, 0.5, 1.0, 5, 10, 50, 100, 500, and 1000 ng/ml were prepared from 10× stocks in methanol, diluted into blank human saliva (two replicates), or in solvent. Table 18 shows a close correlation between input and observed concentrations. FIG. 2 shows the correlation between samples in saliva vs. in solvent. The method described herein is highly sensitive, allowing for the detection of bile acids with a limit of quantitation of 0.001 μmol/L.

TABLE 3

| | Saliva | saliva | |
|---|---|---|---|
| Std (ng/mL) | Replicate-1 | Replicate-2 | Solvent |
| 0.5 | 0.529 | 0.813 | 0.69 |
| 1 | 0.992 | 1.07 | 1.34 |
| 5 | 4.22 | 3.96 | 4.25 |
| 10 | 8.63 | 9.14 | 9.09 |
| 50 | 45.1 | 48.9 | 48.8 |
| 100 | 92.6 | 108 | 102 |
| 500 | 449 | 502 | 510 |
| 1000 | 958 | 1100 | 1010 |

Bile Acid Levels in Healthy Subjects and GERD Patients

Previous studies measuring bile acid (BA) levels in esophageal aspirations indicated that subjects with erosive esophagitis and Barrett's esophagus had significantly elevated BA levels (see, for example, Kauer et al. (1997) Surgery, 122, 874-881; and Nehra et al. (1999) Gut, 44, 598-602). To determine whether the assay described herein provided BA levels similar to those reported in the literature, a pilot study was performed, measuring BA levels in both healthy subjects as well as patients suffering from GERD. Saliva samples were collected from 30 subjects from each group (Normal & GERD). All GERD patients were on proton pump inhibitor (PPI) standard therapy and received a PPI dose the morning of the visit to the clinic. Saliva samples were collected for 2 minutes (min) at the following time points: Fasted state in the morning; Various times after eating a hearty breakfast: 1 hour (hr) after meal; 2 hrs after meal; 3 hrs after meal; 4 hrs after meal.

FIG. 3 shows the time course of BA levels in normal subjects as well as GERD patients on PPI therapy before and up to 4 hours after a hearty meal. The BA levels in both groups are roughly comparable, similar in levels to previously reported values (See, for example, De Corso et al. (2007) Ann. Surgery 245, p. 880-885), with an elevation in salivary bile acid at 1-2 hours after meal. It is likely, that refractory patients experiencing bile acid reflux (patients with was not part of this pilot study.

### Example 2: Determination of Concentrations of Bile Acids in Human Saliva Samples Using a Liquid Chromatographic-Tandem Mass Spectrometric Method

The objective of this study was to determine concentrations of bile acids (cholic acid, chenodeoxycholic acid, glycocholic acid, deoxycholic acid, glycodeoxycholic acid, lithocholic acid, taurodeoxycholic acid, taurocholic acid, glycochenodeoxycholic acid and taurochenodeoxycholic acid) in human saliva samples using a qualified LC-MS/MS method.

Abbreviations

BQL: Below quantitation limit; CV: Coefficient of variation; Dil: Dilution; ID: Identification; IS: Internal standard; LC-MS/MS: Liquid chromatography with tandem mass spectrometry; N: Number of samples; MPA: Mobile phase A; MPB: Mobile phase B; NA: Not applicable; QC: Quality control; SD: Standard deviation; Std: Standard.

Three hundred human saliva samples were received in good condition from Texas Digestive Disease Consultants (Southlake, Tex.). All samples were received frozen, packaged on dry ice, and were stored in a freezer set to maintain a temperature of −80° C. until analysis.

Materials and Methods

Reference and Internal Standards

Reference Standard Cholic acid Manufacturer/Supplier Sigma-Aldrich Batch Number MKBR9198V Storage Conditions Ambient

Reference Standard Chenodeoxycholic acid Manufacturer/Supplier Santa Cruz Biotechnology, Inc. Lot Number K1514 Storage Conditions Ambient

Reference Standard Glycocholic acid hydrate Manufacturer/Supplier Sigma-Aldrich Batch Number SLBH5157V Storage Conditions Ambient

Reference Standard Deoxycholic acid Manufacturer/Supplier Sigma-Aldrich Batch Number BCBN9953V Storage Conditions Ambient

Reference Standard Glycodeoxycholic acid manufacturer/Supplier IsoSciences, LLC Lot Number EH1-2014-028A1 Storage Conditions −20° C., desiccated

Reference Standard Lithocholic acid Manufacturer/Supplier IsoSciences, LLC Lot Number EH1-2014-030A1 Storage Conditions −20° C., desiccated

Reference Standard Sodium taurodeoxycholate hydrate Manufacturer/Supplier Sigma-Aldrich Batch Number SLBJ4610V Storage Conditions Ambient

Reference Standard Taurocholic acid sodium salt hydrate Manufacturer/Supplier Sigma-Aldrich Batch Number SLBH5200V Storage Conditions Ambient

Reference Standard Sodium Glycochenodeoxycholate Manufacturer/Supplier Sigma-Aldrich Batch Number SLBG7615V Storage Conditions Ambient

Reference Standard Sodium Taurochenodeoxycholate Manufacturer/Supplier Sigma-Aldrich Batch Number SLBH9352V Storage Conditions Ambient

Internal Standard Glycocholic Acid-d4 Manufacturer/Supplier C/D/N Isotopes, Inc. Lot Number R376P48 Storage Conditions Ambient

Blank Matrix

Saliva was collected from human volunteers and then pooled. Aliquots of pooled saliva were stripped of endogenous bile acids by treatment with 2 mg/mL of cholestyramine resin (Sigma Lot No. 1425455V) for 1 hour at 37° C. followed by centrifugation. The treatment/centrifugation cycle was repeated four times for a total of five cycles. After the final treatment, the saliva was pooled for use. The cholestyramine-treated saliva was used for preparation of calibration standards and for quality control samples.

Preparation of Calibration Standards

Calibration standards were prepared at concentrations of 0.500, 0.750, 1.00, 2.00, 5.00, 10.0, 50.0, 75.0 and 100 ng/mL cholic acid, glycocholic acid, deoxycholic acid, glycodeoxycholic acid, taurodeoxycholic acid, taurocholic acid, glycochenodeoxycholic acid, taurochenodeoxycholic acid in blank matrix, 5.00, 7.50, 10.0, 20.0, 50.0, 100, 500, 750 and 1000 ng/mL chenodeoxycholic acid in blank matrix and 10.0, 20.0, 50.0, 100, 500, 750 and 1000 ng/mL lithocholic acid in blank matrix. Standards were prepared in small volumes on the day of sample extraction and were analyzed in duplicate in each analytical run.

Preparation of QC Samples

Quality control samples were prepared containing cholic acid, glycocholic acid, deoxycholic acid, glycodeoxycholic acid, taurodeoxycholic acid, taurocholic acid, glycochenodeoxycholic acid, taurochenodeoxycholic acid at 1.50 ng/mL (QC-Low), 8.00 ng/mL (QC-Mid) and 80.0 ng/mL (QC-High) and chenodeoxycholic acid and lithocholic acid at 15.0 ng/mL (QC Low), 80.0 ng/mL (QC-Mid) and 800 ng/mL (QC-High) in blank matrix. Quality control samples were prepared in small volumes on the day of sample extraction.

Sample Extraction

A 100-μL aliquot of sample (calibration standards, quality controls, blanks, and study samples) was transferred into a 96 well plate, according to a pre-defined layout. Three hundred microliters (300 μL) of ice-cold internal standard spiking solution (2 ng/mL glycocholic acid-d4 in acetonitrile) was added to each sample, except for matrix blanks to which 300 μL of acetonitrile was added. The plates were covered, vortex-mixed and then centrifuged for 5 minutes at 3200 rpm. Supernatants (350 μL each) were transferred into the corresponding wells of a clean 96 well plate and evaporated to dryness under nitrogen in a Turbovap set to 40° C. The dried residue in each well was reconstituted with 75 μL of 50:50 (v:v) methanol:water.

Liquid Chromatography (LC) and Mass Spectrometer Conditions

The LC system used was a CTC PAL Autosampler along with Agilent 1260 series pumps. A Hypersil Gold, 1.9 μm column (50×2.1 mm) was used and maintained at 40° C. during analysis. The gradient and mobile phases used are shown below. The flow rate was 0.500 mL/min and the injection volume was 10 μL.

Mobile Phase A: 0.2% (v/v) Formic acid in water; Mobile Phase B: 0.2% (v/v) Formic acid in acetonitrile

Gradient Program:

| Total Time (min) | % MPA | % MPB |
|---|---|---|
| 0.00 | 70 | 30 |
| 0.30 | 50 | 50 |
| 2.27 | 20 | 80 |
| 6.00 | 2 | 98 |
| 7.00 | 2 | 98 |
| 7.10 | 70 | 30 |
| 8.00 | 70 | 30 |

The detector was an Applied Biosystems Sciex API-5500 triple quadrupole mass spectrometer. The instrument was equipped with an electrospray ionization source in positive-ion mode and the analytes were monitored in the multiple-reaction-monitoring scan mode. Q1 and Q3 were operated with unit resolution. The MS/MS transition masses used for the bile acids and internal standard are listed below.

| Compound | Q1 m/z | Q3 m/z |
|---|---|---|
| Cholic acid | 407.1 | 407.1 |
| Chenodeoxycholic acid | 391.2 | 391.2 |
| Glycocholic acid | 464.1 | 74.1 |
| Deoxycholic acid | 391.2 | 391.2 |
| Glycodeoxycholic acid | 448.2 | 74.0 |
| Lithocholic acid | 375.1 | 375.1 |
| Taurodeoxycholic acid | 498.2 | 80.0 |
| Taurocholic acid | 514.2 | 80.0 |
| Glycochenodeoxycholic acid | 448.2 | 74.0 |
| Taurochenodeoxycholic acid | 498.2 | 80.0 |
| Glycocholic acid-d4 | 468.1 | 74.1 |

Data Collection and Analysis

Analyst (Applied Biosystems Sciex) version 1.6 and Aria was used for data acquisition and processing. Descriptive statistics were calculated using Excel (Microsoft).

Run Acceptance Criteria

Data were considered acceptable if the following criteria were met:

At least 75% of the calibration standards are within ±30% of their nominal concentrations.

At least two-thirds of the total number of quality control samples (excluding dilution QCs) and at least 50% of the QC replicates per level are within ±30% of their nominal concentrations. For the dilution QC, at least two-thirds of the replicates are within ±30% of the nominal concentration.

Results

Cholic Acid

Results for cholic acid concentrations in human saliva samples are reported in Table 4. Back-calculated concentrations for the calibration standards are reported in Table 5. Results for batch acceptance quality controls are reported in Table 6.

Chenodeoxycholic Acid

Results for chenodeoxycholic acid concentrations in human saliva samples are reported in Table 7. For some samples, there was no significant separation between chenodeoxycholic acid and deoxycholic acid due to matrix effects. The samples were diluted by a factor of 5 with blank matrix (cholestyramine-treated human saliva) prior to extraction and were reanalyzed in Batch 04. After the diluted samples were analyzed, matrix effects were observed for three samples. The reported concentration for these samples may not accurately reflect true concentration. Back-calculated concentrations for the calibration standards are reported in Table 8. Results for batch acceptance quality controls are reported in Table 9.

Glycocholic Acid

Results for glycocholic acid concentrations in human saliva samples are reported in Table 10. Back-calculated concentrations for the calibration standards are reported in Table 11. Results for batch acceptance quality controls are reported in Table 12.

Deoxycholic Acid

Results for deoxycholic acid concentrations in human saliva samples are reported in Table 13. For some samples, there was no significant separation between chenodeoxycholic acid and deoxycholic acid due to matrix effects. The samples were diluted by a factor of 5 with blank matrix (cholestyramine-treated human saliva) prior to extraction and were reanalyzed in Batch 04. After the diluted samples were analyzed, matrix effects were observed for 18 samples. The reported concentration for these samples may not accurately reflect true concentration. Back-calculated concentrations for the calibration standards are reported in Table 14. Results for batch acceptance quality controls are reported in Table 15.

Glycodeoxycholic Acid

Results for glycodeoxycholic acid concentrations in human saliva samples are reported in Table 16. Back-calculated concentrations for the calibration standards are reported in Table 17. Results for batch acceptance quality controls are reported in Table 18.

Lithocholic Acid

Results for lithocholic acid concentrations in human saliva samples are reported in Table 19. Back-calculated concentrations for the calibration standards are reported in Table 20. Results for batch acceptance quality controls are reported in Table 21.

Taurodeoxycholic Acid

Results for taurodeoxycholic acid concentrations in human saliva samples are reported in Table 22. Back-calculated concentrations for the calibration standards are reported in Table 23. Results for batch acceptance quality controls are reported in Table 24.

Taurocholic Acid

Results for taurocholic acid concentrations in human saliva samples are reported in Table 25. Back-calculated concentrations for the calibration standards are reported in Table 26. Results for batch acceptance quality controls are reported in Table 27.

Glycochenodeoxycholic Acid

Results for glycochenodeoxycholic acid concentrations in human saliva samples are reported in Table 28. Back-calculated concentrations for the calibration standards are reported in Table 29. Results for batch acceptance quality controls are reported in Table 30.

Taurochenodeoxycholic Acid

Results for taurochenodeoxycholic acid concentrations in human saliva samples are reported in Table 31. Back-calculated concentrations for the calibration standards are reported in Table 32. Results for batch acceptance quality controls are reported in Table 33.

Analytes: Cholic acid
Chenodeoxycholic acid
Glycocholic acid
Deoxycholic acid
Glycodeoxycholic acid
Lithocholic acid
Taurodeoxycholic acid
Taurocholic acid
Glycochenodeoxycholic acid
Taurochenodeoxycholic acid
Matrix: Human saliva
Internal Standard: Glycocholic acid-d4
Matrix for Standards, QCs and Blanks: Cholestyramine-treated human saliva, pooled
Extraction Volume: 100 μL
Extraction Procedure: Protein precipitation
Calibration Ranges

Cholic acid 0.500 to 100 ng/mL

Chenodeoxycholic acid 5.00 to 1000 ng/mL

Glycocholic acid 0.500 to 100 ng/mL

Deoxycholic acid 0.500 to 100 ng/mL

Glycodeoxycholic acid 0.500 to 100 ng/mL

Lithocholic acid 10.0 to 1000 ng/mL

Taurodeoxycholic acid 0.500 to 100 ng/mL

Taurocholic acid 0.500 to 100 ng/mL

Glycochenodeoxycholic acid 0.500 to 100 ng/mL

Taurochenodeoxycholic acid 0.500 to 100 ng/mL
Quality Control Concentrations

Cholic acid 1.50, 8.00 and 80.0 ng/mL

Chenodeoxycholic acid 15.0, 80.0, 800 and 1000 ng/mL

Glycocholic acid 1.50, 8.00 and 80.0 ng/mL

Deoxycholic acid 1.50, 8.00, 80.0 and 100 ng/mL

Glycodeoxycholic acid 1.50, 8.00 and 80.0 ng/mL

Lithocholic acid 15.0, 80.0 and 800 ng/mL

Taurodeoxycholic acid 1.50, 8.00 and 80.0 ng/mL

Taurocholic acid 1.50, 8.00 and 80.0 ng/mL

Glycochenodeoxycholic acid 1.50, 8.00 and 80.0 ng/mL

Taurochenodeoxycholic acid 1.50, 8.00 and 80.0 ng/mL
LC Conditions: CTC PAL HTS-XT autosampler
   with Agilent 1260 series pumps
   Hypersil Gold, 1.9 μm column (50×2.1 mm)
MS Conditions: API-5500
   Electrospray ionization (positive-ion mode)
   Multiple-reaction-monitoring scan mode
Regression, Weighting: Linear, 1/x²
Acceptance Criteria: Within ±30% from nominal concentrations

TABLE 4
Cholic Acid: Concentrations in Human Saliva Samples

| | Concentration (ng/mL) | | | | |
|---|---|---|---|---|---|
| | Time Point (hour post breakfast) | | | | |
| Subject No. | Pre | 1 | 2 | 3 | 4 |
| 001-0001 | BQL | BQL | BQL | BQL | BQL |
| 001-0002 | 3.09 | 1.37 | 0.767 | BQL | BQL |
| 001-0003 | 1.33 | BQL | BQL | BQL | BQL |
| 001-0004 | 1.61 | BQL | BQL | BQL | BQL |
| 001-0005 | BQL | BQL | BQL | BQL | BQL |
| 001-0006 | BQL | BQL | BQL | BQL | BQL |
| 001-0007 | BQL | BQL | BQL | BQL | BQL |
| 001-0008 | 3.51 | 0.791 | 1.33 | 0.966 | 0.989 |
| 001-0009 | 2.06 | 1.09 | 1.48 | 1.54 | 1.70 |
| 001-0010 | 0.795 | BQL | BQL | BQL | BQL |
| 001-0011 | 1.58 | BQL | BQL | BQL | BQL |
| 001-0012 | 0.817 | 0.579 | 0.864 | 0.910 | 0.706 |
| 001-0013 | BQL | BQL | BQL | BQL | BQL |
| 001-0014 | 3.20 | 0.517 | BQL | 0.525 | BQL |
| 001-0015 | BQL | BQL | BQL | BQL | BQL |
| 001-0016 | 1.04 | BQL | BQL | BQL | BQL |
| 001-0017 | 3.63 | 1.02 | BQL | BQL | BQL |
| 001-0018 | BQL | BQL | BQL | BQL | BQL |
| 001-0019 | 2.13 | 1.49 | 2.11 | 2.27 | 1.84 |
| 001-0020 | BQL | BQL | BQL | BQL | BQL |
| 001-0021 | BQL | BQL | BQL | BQL | BQL |
| 001-0022 | 0.573 | BQL | BQL | BQL | BQL |
| 001-0023 | BQL | BQL | BQL | BQL | BQL |
| 001-0024 | 0.526 | BQL | BQL | BQL | BQL |
| 001-0025 | BQL | BQL | BQL | BQL | BQL |
| 001-0026 | BQL | 0.838 | 0.839 | 1.79 | 5.27 |
| 001-0027 | BQL | BQL | BQL | BQL | BQL |
| 001-0028 | 0.857 | BQL | BQL | BQL | BQL |
| 001-0029 | BQL | BQL | BQL | BQL | BQL |
| 001-0030 | BQL | BQL | BQL | BQL | BQL |
| 001-0031 | BQL | BQL | BQL | BQL | BQL |
| 001-0032 | 0.518 | BQL | 0.558 | 0.503 | 0.594 |
| 001-0033 | 0.956 | 0.843 | BQL | BQL | BQL |
| 001-0034 | 0.753 | 0.510 | BQL | BQL | BQL |
| 001-0035 | 1.34 | BQL | BQL | BQL | BQL |
| 001-0036 | 2.38 | 1.51 | 2.49 | 2.07 | 1.53 |
| 001-0037 | BQL | BQL | BQL | BQL | BQL |
| 001-0038 | 1.94 | 0.545 | 0.934 | 0.744 | BQL |
| 001-0039 | BQL | BQL | BQL | BQL | BQL |
| 001-0040 | BQL | BQL | BQL | BQL | BQL |
| 001-0041 | BQL | BQL | BQL | BQL | BQL |
| 001-0042 | 2.23 | BQL | 1.09 | BQL | BQL |
| 001-0043 | BQL | BQL | BQL | BQL | BQL |
| 001-0044 | BQL | 2.25 | 1.55 | 0.945 | 0.558 |
| 001-0045 | 4.08 | 1.25 | 0.972 | 0.592 | BQL |
| 001-0046 | 3.51 | 1.48 | 1.05 | 4.01 | 10.3 |
| 001-0047 | BQL | BQL | BQL | BQL | BQL |
| 001-0048 | BQL | BQL | BQL | BQL | BQL |
| 001-0049 | 1.15 | 3.33 | 3.15 | 3.08 | 2.50 |
| 001-0050 | BQL | BQL | BQL | BQL | BQL |
| 001-0051 | BQL | BQL | BQL | BQL | BQL |
| 001-0052 | 2.21 | BQL | BQL | BQL | BQL |
| 001-0053 | 0.623 | BQL | 0.817 | 0.892 | BQL |
| 001-0054 | BQL | BQL | BQL | BQL | BQL |
| 001-0055 | 0.517 | BQL | BQL | BQL | BQL |
| 001-0056 | 1.55 | BQL | BQL | BQL | BQL |
| 001-0057 | BQL | BQL | 0.951 | BQL | BQL |
| 001-0058 | 2.24 | 0.573 | BQL | BQL | BQL |
| 001-0059 | 4.88 | 4.65 | 1.07 | 0.568 | BQL |
| 001-0060 | 1.06 | 0.874 | 1.03 | BQL | 0.530 |

BQL: Below quantitation limit (0.500 ng/mL)

TABLE 5
Cholic Acid: Calibration Standard Back-Calculated Concentrations in Cholestyramine-Treated Human Saliva

| | Concentration (ng/mL) | | | | | | | | |
|---|---|---|---|---|---|---|---|---|---|
| Analytical | Std-1 | Std-2 | Std-3 | Std-4 | Std-5 | Std-6 | Std-7 | Std-8 | Std-9 |
| Run | 0.500 | 0.750 | 1.00 | 2.00 | 5.00 | 10.0 | 50.0 | 75.0 | 100 |
| B01 | 0.449 | 0.701 | 1.03 | 2.02 | 4.54 | 8.56 | 44.8 | 71.9 | 94.3 |
| | 0.515 | 0.800 | 1.05 | 2.33 | 5.00 | 10.3 | 60.7 | 75.6 | 99.6 |
| B02 | 0.486 | 0.753 | 0.985 | 2.02 | 4.89 | 9.85 | 50.7 | 76.4 | 93.3 |
| | 0.481 | 0.782 | 1.05 | 2.09 | 5.23 | 10.0 | 50.5 | 75.2 | 93.5 |
| B03 | 0.521 | 0.776 | 0.973 | 2.00 | 4.92 | 9.49 | 48.6 | 73.9 | 101 |
| | 0.506 | 0.730 | 0.924 | 1.89 | 5.31 | 10.5 | 50.1 | 75.2 | 108 |
| Mean | 0.493 | 0.757 | 1.00 | 2.06 | 4.98 | 9.78 | 50.9 | 74.7 | 98.3 |
| SD | 0.0267 | 0.0366 | 0.0502 | 0.148 | 0.275 | 0.695 | 5.28 | 1.59 | 5.78 |
| % CV | 5.4 | 4.8 | 5.0 | 7.2 | 5.5 | 7.1 | 10.4 | 2.1 | 5.9 |
| % Bias | −1.4 | 0.9 | 0.2 | 2.9 | −0.4 | −2.2 | 1.8 | −0.4 | −1.7 |
| n | 6 | 6 | 6 | 6 | 6 | 6 | 6 | 6 | 6 |

TABLE 6
Cholic Acid: Batch Acceptance Quality Controls
(Cholestyramine-Treated Human Saliva)

| | Concentration (ng/mL) | | |
|---|---|---|---|
| Analytical | QC-Low | QC-Mid | QC-High |
| Run | 1.50 | 8.00 | 80.0 |
| B01 | 1.07 | 5.91 | 58.5 |
| | 1.04 | 5.73 | 62.4 |
| | 1.14^{a} | 6.14 | 61.2 |
| B02 | 1.17 | 6.07 | 63.6 |
| | 1.18 | 6.14 | 63.0 |
| | 1.27 | 6.00 | 58.0 |
| B03 | 1.09 | 5.81 | 61.8 |
| | 1.06 | 5.57 | 60.0 |
| | 1.16 | 5.73 | 61.7 |
| Mean | 1.13 | 5.90 | 61.1 |
| SD | 0.0732 | 0.203 | 1.94 |
| % CV | 6.5 | 3.4 | 3.2 |
| % Bias | −24.6 | −26.3 | −23.6 |
| n | 9 | 9 | 9 |

^{a}Failed to meet acceptance criteria (within ±30% from nominal concentration); included in statistics.

TABLE 7
Chenodeoxycholic Acid: Concentrations in Human Saliva Samples

| | Concentration (ng/mL) | | | | |
|---|---|---|---|---|---|
| | Time Point (hour post breakfast) | | | | |
| Subject No. | Pre | 1 | 2 | 3 | 4 |
| 001-0001 | BQL2 | BQL2 | BQL | BQL | BQL |
| 001-0002 | BQL2 | BQL2 | BQL | BQL | BQL |
| 001-0003 | BQL2 | BQL2 | BQL | BQL | BQL |
| 001-0004 | BQL2 | BQL2 | BQL | BQL | BQL |
| 001-0005 | BQL | BQL | BQL | BQL | BQL |
| 001-0006 | BQL2 | BQL | BQL | BQL | BQL |
| 001-0007 | BQL2 | BQL | BQL | BQL | BQL |
| 001-0008 | 205^{a, b} | BQL | BQL | BQL | BQL |
| 001-0009 | BQL2 | BQL2 | BQL2 | BQL | BQL |
| 001-0010 | BQL2 | BQL | BQL | BQL | BQL |
| 001-0011 | BQL2 | BQL | BQL2 | BQL | BQL |
| 001-0012 | BQL2 | BQL2 | BQL2 | BQL | BQL |
| 001-0013 | BQL2 | BQL | BQL | BQL | BQL |
| 001-0014 | BQL2 | BQL | BQL | BQL | BQL |
| 001-0015 | BQL | BQL | BQL | BQL | BQL |
| 001-0016 | BQL2 | BQL | BQL | BQL | BQL |
| 001-0017 | BQL2 | BQL | BQL | BQL | BQL |
| 001-0018 | BQL | BQL | BQL | BQL | BQL |
| 001-0019 | BQL2 | BQL | BQL | BQL | BQL |
| 001-0020 | BQL2 | BQL | BQL | BQL | BQL |
| 001-0021 | BQL | BQL | BQL | BQL | BQL |
| 001-0022 | BQL2 | BQL | BQL | BQL | BQL |
| 001-0023 | BQL | BQL | BQL | BQL | BQL |
| 001-0024 | BQL2 | BQL2 | BQL2 | BQL2 | BQL2 |
| 001-0025 | BQL2 | BQL2 | BQL | BQL | BQL |
| 001-0026 | BQL2 | BQL2 | BQL2 | BQL | BQL |
| 001-0027 | BQL2 | BQL2 | BQL | BQL | BQL |
| 001-0028 | BQL2 | BQL | BQL | BQL | BQL |
| 001-0029 | BQL | BQL | BQL | BQL | BQL |
| 001-0030 | BQL | BQL | BQL | BQL | BQL |
| 001-0031 | BQL2 | BQL | BQL | BQL | BQL |
| 001-0032 | BQL | BQL | BQL | BQL | BQL |
| 001-0033 | BQL2 | BQL2 | BQL2 | BQL2 | BQL |
| 001-0034 | 185^{a, b} | BQL2 | BQL | BQL | BQL |
| 001-0035 | BQL | BQL | BQL | BQL | BQL |
| 001-0036 | BQL2 | BQL | BQL | BQL | BQL |
| 001-0037 | BQL2 | BQL | BQL | BQL | BQL |
| 001-0038 | 224^{a, b} | BQL | BQL2 | BQL | BQL |
| 001-0039 | BQL | BQL | BQL | BQL | BQL |
| 001-0040 | BQL2 | BQL | BQL | BQL | BQL |
| 001-0041 | BQL2 | BQL2 | BQL | BQL | BQL |
| 001-0042 | BQL2 | BQL | BQL | BQL | BQL |
| 001-0043 | BQL | BQL | BQL | BQL | BQL |
| 001-0044 | BQL | BQL | BQL | BQL | BQL |
| 001-0045 | BQL2 | BQL | BQL | BQL | BQL |
| 001-0046 | BQL2 | BQL | BQL | BQL | 17.6 |
| 001-0047 | BQL2 | BQL2 | BQL | BQL | BQL |
| 001-0048 | BQL2 | BQL | BQL | BQL | BQL |
| 001-0049 | BQL | BQL | BQL | BQL | BQL |
| 001-0050 | BQL | BQL | BQL | BQL | BQL |
| 001-0051 | BQL2 | BQL | BQL | BQL | BQL |
| 001-0052 | BQL2 | BQL | BQL | BQL | BQL |
| 001-0053 | BQL | BQL | BQL | BQL | BQL |
| 001-0054 | BQL2 | BQL | BQL | BQL | BQL |
| 001-0055 | BQL | BQL | BQL | BQL | BQL |
| 001-0056 | BQL2 | BQL2 | BQL2 | BQL2 | BQL |
| 001-0057 | BQL | BQL | BQL | BQL | BQL |
| 001-0058 | BQL2 | BQL | BQL | BQL | BQL |
| 001-0059 | BQL2 | BQL2 | BQL | BQL | BQL |
| 001-0060 | BQL2 | BQL | BQL | BQL | BQL |

BQL: Below quantitation limit (5.00 ng/mL)
BQL2: Below quantitation limit (25.0 ng/mL); Batch 04, diluted 5x with blank matrix prior to extraction.
^{a}Reassay result from Batch B04 (sample diluted 5x with blank matrix prior to extraction).
The reassay result does not match original result (BQL).
^{b}Matrix effects: no significant separation between chenodeoxycholic acid and deoxycholic acid. Reported concentration may not accurately reflect true concentration.

TABLE 8
Chenodeoxycholic Acid: Calibration Standard Back-Calculated Concentrations in
Cholestyramine-Treated Human Saliva

| | Concentration (ng/mL) | | | | | | | | |
|---|---|---|---|---|---|---|---|---|---|
| Analytical | Std-1 | Std-2 | Std-3 | Std-4 | Std-5 | Std-6 | Std-7 | Std-8 | Std-9 |
| Run | 5.00 | 7.50 | 10.0 | 20.0 | 50.0 | 100 | 500 | 750 | 1000 |
| B01 | 4.43 | 6.81 | 10.8 | 24.4 | 55.7 | 98.1 | 360 | 652 | 861 |
| | 4.77 | 7.48 | 11.2 | 23.9 | 53.2 | 119 | NR | 709 | 896 |
| B02 | 4.83 | 7.37 | 9.67 | 19.7 | 55.0 | 111 | 472 | 753 | 912 |
| | 4.85 | 7.69 | 11.0 | 19.6 | 57.6 | 108 | 481 | 709 | 806 |
| B03 | 5.23 | 7.41 | 10.4 | 22.5 | 55.3 | 107 | 490 | 667 | 844 |
| | 4.64 | 6.96 | 9.61 | 21.4 | 58.6 | 112 | 453 | 666 | 935 |
| B04 | 4.19 | 6.42 | 10.3 | 21.2 | 53.7 | 99.5 | 410 | 759 | 816 |
| | 5.51 | 7.27 | 11.2 | 24.4 | 59.5 | 113 | 479 | 765 | 782 |
| Mean | 4.81 | 7.18 | 10.5 | 22.1 | 56.1 | 108 | 449 | 710 | 857 |
| SD | 0.419 | 0.416 | 0.638 | 1.97 | 2.28 | 6.98 | 47.6 | 45.4 | 54.4 |
| % CV | 8.7 | 5.8 | 6.1 | 8.9 | 4.1 | 6.4 | 10.6 | 6.4 | 6.4 |
| % Bias | −3.9 | −4.3 | 5.2 | 10.7 | 12.2 | 8.5 | −10.1 | −5.3 | −14.4 |
| n | 8 | 8 | 8 | 8 | 8 | 8 | 7 | 8 | 8 |

NR: Not reported

TABLE 9
Chenodeoxycholic Acid: Batch Acceptance Quality
Controls (Cholestyramine-Treated Human Saliva)

Concentration (ng/mL)

| Analytical | QC-Low | QC-Mid | QC-High | QC-Dil |
|---|---|---|---|---|
| Run | 15.0 | 80.0 | 800 | 1000 |
| B01 | 15.4 | 85.7 | 727 | NA |
| | 14.8 | 80.9 | 681 | NA |
| | 17.5 | 95.7 | 725 | NA |
| B02 | 16.4 | 85.2 | 744 | NA |
| | 16.8 | 91.5 | 783 | NA |
| | 18.2 | 84.7 | 701 | NA |
| B03 | 14.9 | 79.6 | 751 | NA |
| | 15.7 | 77.2 | 708 | NA |
| | 16.1 | 79.6 | 697 | NA |
| B04 | 16.2 | 103 | 839 | 1090 |
| | 15.9 | 91.1 | 703 | 1220 |
| | 17.8 | 91.0 | 805 | 1040 |
| Mean | 16.3 | 87.1 | 739 | 1120 |
| SD | 1.09 | 7.61 | 48.3 | 92.9 |
| % CV | 6.7 | 8.7 | 6.5 | 8.3 |
| % Bias | 8.7 | 8.9 | −7.7 | 11.7 |
| n | 12 | 12 | 12 | 3 |

^{a}Diluted by a factor of 5 for analysis.

TABLE 10
Glycocholic Acid: Concentrations in Human Saliva Samples;
BQL: Below quantitation limit (0.500 ng/mL)

| | Concentration (ng/mL) | | | | |
|---|---|---|---|---|---|
| | Time Point (hour post breakfast) | | | | |
| Subject No. | Pre | 1 | 2 | 3 | 4 |
| 001-0001 | BQL | BQL | BQL | BQL | BQL |
| 001-0002 | BQL | BQL | BQL | BQL | BQL |
| 001-0003 | 0.556 | BQL | BQL | BQL | BQL |
| 001-0004 | BQL | BQL | BQL | BQL | BQL |
| 001-0005 | BQL | BQL | BQL | BQL | BQL |
| 001-0006 | 0.551 | BQL | BQL | BQL | BQL |
| 001-0007 | BQL | BQL | BQL | BQL | BQL |
| 001-0008 | 3.13 | 1.94 | 2.40 | 1.92 | 2.07 |
| 001-0009 | 0.741 | 1.49 | 2.86 | 3.19 | 3.77 |
| 001-0010 | BQL | BQL | BQL | BQL | BQL |
| 001-0011 | BQL | BQL | BQL | BQL | BQL |
| 001-0012 | BQL | 1.06 | 1.19 | 1.14 | 0.772 |
| 001-0013 | BQL | BQL | BQL | BQL | BQL |
| 001-0014 | BQL | 0.633 | 1.49 | 1.03 | 0.785 |
| 001-0015 | BQL | BQL | BQL | BQL | BQL |
| 001-0016 | 0.588 | BQL | BQL | BQL | BQL |
| 001-0017 | BQL | 0.985 | BQL | BQL | BQL |
| 001-0018 | 0.593 | 0.921 | 0.793 | 0.692 | 0.721 |
| 001-0019 | BQL | 0.545 | 0.674 | 1.15 | 0.877 |
| 001-0020 | BQL | BQL | BQL | BQL | BQL |
| 001-0021 | BQL | BQL | BQL | BQL | BQL |
| 001-0022 | 0.512 | 1.07 | 1.34 | 0.776 | 0.551 |
| 001-0023 | BQL | BQL | BQL | BQL | BQL |
| 001-0024 | 2.21 | 0.570 | BQL | BQL | BQL |
| 001-0025 | BQL | BQL | BQL | BQL | BQL |
| 001-0026 | 1.35 | 1.35 | 1.13 | 0.893 | 0.902 |
| 001-0027 | BQL | BQL | BQL | BQL | BQL |
| 001-0028 | 2.00 | 0.971 | 1.07 | 1.40 | 0.850 |
| 001-0029 | BQL | BQL | 0.618 | BQL | BQL |
| 001-0030 | BQL | BQL | BQL | BQL | BQL |
| 001-0031 | BQL | BQL | BQL | BQL | BQL |
| 001-0032 | BQL | BQL | 1.11 | 0.589 | 0.590 |
| 001-0033 | BQL | BQL | BQL | BQL | BQL |
| 001-0034 | 0.664 | 0.710 | 0.654 | BQL | 0.642 |
| 001-0035 | 0.670 | BQL | BQL | BQL | BQL |
| 001-0036 | 3.73 | 5.88 | 11.0 | 8.23 | 3.30 |
| 001-0037 | BQL | BQL | BQL | BQL | BQL |
| 001-0038 | 1.03 | 0.540 | 1.11 | 0.599 | BQL |
| 001-0039 | BQL | BQL | BQL | BQL | BQL |
| 001-0040 | 1.13 | 0.734 | 0.621 | 0.539 | BQL |
| 001-0041 | BQL | BQL | BQL | BQL | BQL |
| 001-0042 | BQL | BQL | BQL | BQL | BQL |
| 001-0043 | BQL | BQL | 0.522 | BQL | BQL |
| 001-0044 | 0.679 | 0.780 | 0.901 | 1.26 | 1.20 |
| 001-0045 | BQL | 1.22 | 1.41 | 1.25 | 1.09 |
| 001-0046 | BQL | BQL | BQL | BQL | BQL |
| 001-0047 | 0.641 | 1.30 | 0.989 | 0.846 | BQL |
| 001-0048 | BQL | BQL | 0.837 | 0.521 | BQL |
| 001-0049 | BQL | 0.785 | 1.17 | 1.30 | 1.04 |
| 001-0050 | 1.02 | 1.58 | 1.51 | 1.17 | 0.795 |
| 001-0051 | BQL | 0.585 | 0.928 | 0.536 | BQL |
| 001-0052 | BQL | BQL | 0.505 | BQL | BQL |
| 001-0053 | 0.649 | 1.43 | 1.30 | 0.933 | BQL |
| 001-0054 | BQL | BQL | 0.509 | 0.502 | BQL |
| 001-0055 | 1.91 | 1.50 | 1.12 | 0.869 | 0.843 |
| 001-0056 | BQL | 0.618 | BQL | BQL | BQL |
| 001-0057 | 0.801 | 1.93 | 3.05 | 1.66 | 1.78 |
| 001-0058 | 2.11 | 0.787 | 1.02 | 0.530 | BQL |
| 001-0059 | BQL | 0.576 | BQL | BQL | BQL |
| 001-0060 | 2.40 | 5.64 | 4.47 | 1.80 | 4.87 |

TABLE 11
Glycocholic Acid: Calibration Standard Back-Calculated Concentrations in
Cholestyramine-Treated Human Saliva

| | Concentration (ng/mL) | | | | | | | | |
|---|---|---|---|---|---|---|---|---|---|
| Analytical | Std-1 | Std-2 | Std-3 | Std-4 | Std-5 | Std-6 | Std-7 | Std-8 | Std-9 |
| Run | 0.500 | 0.750 | 1.00 | 2.00 | 5.00 | 10.0 | 50.0 | 75.0 | 100 |
| B01 | 0.492 | 0.727 | 1.04 | 2.06 | 4.78 | 9.28 | 44.7 | 76.2 | 98.4 |
| | 0.495 | 0.709 | 1.06 | 2.29 | 4.93 | 9.53 | 56.8 | 74.0 | 99.2 |
| B02 | 0.498 | 0.739 | 0.980 | 1.99 | 4.92 | 9.75 | 50.3 | 76.6 | 98.1 |
| | 0.486 | 0.778 | 1.05 | 2.03 | 5.15 | 9.85 | 50.3 | 76.3 | 96.6 |
| B03 | 0.514 | 0.750 | 0.969 | 2.08 | 5.06 | 9.73 | 48.4 | 76.5 | 99.3 |
| | 0.498 | 0.736 | 0.980 | 1.99 | 5.28 | 10.0 | 49.7 | 72.4 | 103 |
| Mean | 0.497 | 0.740 | 1.01 | 2.07 | 5.02 | 9.69 | 50.0 | 75.3 | 99.1 |
| SD | 0.00939 | 0.0232 | 0.0410 | 0.112 | 0.180 | 0.253 | 3.93 | 1.73 | 2.14 |
| % CV | 1.9 | 3.1 | 4.1 | 5.4 | 3.6 | 2.6 | 7.9 | 2.3 | 2.2 |
| % Bias | −0.6 | −1.4 | 1.3 | 3.7 | 0.4 | −3.1 | 0.1 | 0.4 | −0.9 |
| n | 6 | 6 | 6 | 6 | 6 | 6 | 6 | 6 | 6 |

TABLE 12
Glycocholic Acid: Batch Acceptance Quality Controls
(Cholestyramine-Treated Human Saliva)

| | Concentration (ng/mL) | | |
|---|---|---|---|
| Analytical | QC-Low | QC-Mid | QC-High |
| Run | 1.50 | 8.00 | 80.0 |
| B01 | 1.48 | 7.65 | 77.6 |
| | 1.53 | 7.89 | 81.2 |
| | 1.57 | 7.75 | 81.2 |
| B02 | 1.51 | 7.69 | 82.7 |
| | 1.50 | 7.82 | 79.9 |
| | 1.51 | 7.78 | 79.5 |
| B03 | 1.51 | 7.91 | 79.9 |
| | 1.47 | 7.59 | 81.0 |
| | 1.56 | 7.63 | 80.9 |
| Mean | 1.52 | 7.75 | 80.4 |
| SD | 0.0332 | 0.114 | 1.43 |
| % CV | 2.2 | 1.5 | 1.8 |
| % Bias | 1.0 | −3.2 | 0.5 |
| n | 9 | 9 | 9 |

TABLE 13
Deoxycholic Acid: Concentrations in Human Saliva Samples

| | Concentration (ng/mL) | | | | |
|---|---|---|---|---|---|
| | Time Point (hour post breakfast) | | | | |
| Subject No. | Pre | 1 | 2 | 3 | 4 |
| 001-0001 | 117^{a} | 22.3^{a} | 25.0 | 19.5 | 11.7 |
| 001-0002 | 123^{a} | 7.55^{a} | 11.9 | 4.76 | 4.03 |
| 001-0003 | 165^{a,b} | 10.6^{a} | 19.2 | 13.4 | 7.20 |
| 001-0004 | 111^{a,b} | 22.1^{a} | 21.2 | 8.70 | 13.8 |
| 001-0005 | BQL | BQL | BQL | 0.685 | 1.42 |
| 001-0006 | 17.5^{a} | 6.18 | 1.60 | 2.48 | 1.29 |
| 001-0007 | 166^{a,b} | 21.7 | 24.7 | 5.66 | 4.81 |
| 001-0008 | 73.3^{a,b} | 24.6 | 15.0 | 7.05 | 6.86 |
| 001-0009 | 117^{a,b} | 11.6^{a} | 22.8^{a} | 28.3 | 27.8 |
| 001-0010 | 115^{a} | 22.2 | 10.4 | 7.07 | 7.09 |
| 001-0011 | 157^{a} | 32.6 | 6.07^{a} | 9.25 | 8.47 |
| 001-0012 | 141^{a,b} | 24.4^{a} | 23.7^{a} | 32.8 | 11.1 |
| 001-0013 | 96.8^{a} | 13.2 | 5.21 | 5.60 | 1.11 |
| 001-0014 | 133^{a} | 20.1 | 17.1 | 17.2 | 10.4 |
| 001-0015 | BQL | BQL | BQL | BQL | BQL |
| 001-0016 | 84.0^{a} | 15.3 | 10.1 | 2.21 | 3.89 |
| 001-0017 | 21.0^{a} | 5.57 | 2.01 | 1.92 | 3.11 |
| 001-0018 | 1.19 | 0.530 | 0.533 | BQL | BQL |
| 001-0019 | 66.0^{a} | 12.0 | 15.9 | 15.2 | 6.95 |
| 001-0020 | 30.1^{a} | 2.17 | 2.55 | 1.03 | 0.593 |
| 001-0021 | 1.04 | 0.610 | 0.578 | 0.599 | 0.568 |
| 001-0022 | 2.98^{a} | 12.2 | 15.5 | 5.64 | 4.01 |
| 001-0023 | 27.0 | 2.66 | 2.55 | 1.65 | 0.937 |
| 001-0024 | 132^{a,b} | 85.1^{a} | 35.4^{a} | 5.25^{a} | 16.7^{a} |
| 001-0025 | 71.7^{a} | 9.12^{a} | 18.0 | 8.86 | 1.09 |
| 001-0026 | 161^{a,b} | 35.8^{a} | 24.0^{a} | 30.4 | 30.7 |
| 001-0027 | 146^{a,b} | 19.4 | 24.2 | 23.8 | 19.3 |
| 001-0028 | 68.6^{a,b} | 15.9 | 9.65 | 7.32 | 4.88 |
| 001-0029 | 1.42 | 0.859 | 0.833 | 0.566 | 1.05 |
| 001-0030 | 1.15 | BQL | BQL | BQL | BQL |
| 001-0031 | 126^{a,b} | 22.1 | 31.0 | 33.8 | 26.6 |
| 001-0032 | 2.57 | 2.37 | 3.59 | 2.97 | 2.65 |
| 001-0033 | 126^{a,b} | 24.7^{a} | 19.5^{a} | 10.8^{a} | 19.5 |
| 001-0034 | 74.6^{a,b} | 33.3^{a} | 34.2 | 20.8 | 25.3 |
| 001-0035 | 0.842 | BQL | BQL | BQL | BQL |
| 001-0036 | 138^{a} | 10.9 | 13.3 | 6.99 | 5.50 |
| 001-0037 | 18.7^{a} | 5.55 | 4.73 | 4.23 | 4.90 |
| 001-0038 | 99.3^{a,b} | 20.3 | 15.4^{a} | 18.0 | 8.72 |
| 001-0039 | 1.04 | BQL | 0.814 | 0.789 | 0.837 |
| 001-0040 | 204^{a} | 30.6 | 20.7 | 13.3 | 11.4 |
| 001-0041 | 117^{a,b} | 12.1^{a} | 31.6 | 20.5 | 14.6 |
| 001-0042 | 100^{a} | 7.74 | 8.81 | 4.89 | 3.68 |
| 001-0043 | BQL | BQL | BQL | BQL | BQL |
| 001-0044 | BQL | 0.915 | 0.902 | 0.999 | 0.788 |
| 001-0045 | 142^{a} | 30.7 | 37.4 | 16.9 | 15.1 |
| 001-0046 | 73.1^{a} | 7.10 | 5.31 | 10.3 | 14.0 |
| 001-0047 | 179^{a} | 19.4^{a} | 21.0 | 23.1 | 16.6 |
| 001-0048 | 197^{a,b} | 12.6 | 6.11 | 4.12 | 5.42 |
| 001-0049 | 0.536 | 0.579 | 0.925 | 0.751 | 0.897 |
| 001-0050 | BQL | BQL | BQL | BQL | BQL |
| 001-0051 | 17.0^{a} | 3.60 | 4.90 | 2.97 | 3.07 |
| 001-0052 | 22.2 | 1.20 | 1.73 | 1.43 | 0.966 |
| 001-0053 | 2.34 | BQL | 0.507 | 0.538 | BQL |
| 001-0054 | 88.3^{a} | 8.92 | 14.2 | 6.53 | 2.58 |
| 001-0055 | 4.75 | 1.09 | 3.66 | 1.70 | 3.01 |
| 001-0056 | 109^{a,b} | 25.7^{a} | 20.2^{a} | 10.9^{a} | 25.4 |
| 001-0057 | BQL | BQL | 0.581 | BQL | BQL |
| 001-0058 | 132^{a} | 19.4 | 15.5 | 8.21 | 2.01 |
| 001-0059 | 169^{a,b} | 26.3^{a} | 8.59 | 10.5 | 9.40 |
| 001-0060 | 36.0^{a} | 7.56 | 11.2 | 1.65 | 1.66 |

BQL: Below quantitation limit (0.500 ng/mL)
^{a}Reassay result from Batch B04 (sample diluted 5x with blank matrix prior to extraction) due to matrix effects in original analysis.
^{b}Matrix effects: no significant separation between chenodeoxycholic acid and deoxycholic acid. Reported concentration may not accurately reflect true concentration.

TABLE 14
Deoxycholic Acid: Calibration Standard Back-Calculated Concentrations in
Cholestyramine-Treated Human Saliva

| | Concentration (ng/mL) | | | | | | | | |
|---|---|---|---|---|---|---|---|---|---|
| Analytical | Std-1 | Std-2 | Std-3 | Std-4 | Std-5 | Std-6 | Std-7 | Std-8 | Std-9 |
| Run | 0.500 | 0.750 | 1.00 | 2.00 | 5.00 | 10.0 | 50.0 | 75.0 | 100 |
| B01 | 0.507 | 0.666 | 1.14 | 2.24 | 4.90 | 9.11 | 37.5 | 75.5 | 100 |
| | 0.440 | 0.796 | 1.01 | 2.30 | 4.95 | 10.5 | NR | 80.3 | 97.2 |
| B02 | 0.542 | 0.678 | 0.966 | 1.83 | 4.65 | 9.16 | 51.3 | 77.6 | 105 |
| | 0.511 | 0.762 | 0.955 | 2.12 | 5.01 | 10.2 | 52.2 | 78.7 | 100 |
| B03 | 0.571 | 0.765 | 0.982 | 2.13 | 4.98 | 9.53 | 47.5 | 74.5 | 103 |
| | 0.457 | 0.692 | 0.951 | 1.98 | 5.08 | 10.3 | 48.4 | 76.0 | 106 |
| B04 | 0.452 | 0.655 | 1.02 | 2.01 | 5.24 | 9.66 | 42.7 | 83.1 | 90.6 |
| | 0.521 | 0.794 | 1.10 | 2.21 | 5.27 | 10.8 | 47.0 | 78.0 | 89.4 |
| Mean | 0.500 | 0.726 | 1.02 | 2.10 | 5.01 | 9.91 | 46.7 | 78.0 | 98.9 |
| SD | 0.0466 | 0.0591 | 0.0698 | 0.155 | 0.197 | 0.631 | 5.09 | 2.79 | 6.20 |
| % CV | 9.3 | 8.1 | 6.9 | 7.4 | 3.9 | 6.4 | 10.9 | 3.6 | 6.3 |
| % Bias | 0.0 | −3.2 | 1.5 | 5.1 | 0.2 | −0.9 | −6.7 | 4.0 | −1.1 |
| n | 8 | 8 | 8 | 8 | 8 | 8 | 7 | 8 | 8 |

NR: Not reported

TABLE 15
Deoxycholic Acid: Batch Acceptance Quality Controls
(Cholestyramine-Treated Human Saliva)

Concentration (ng/mL)

| | QC-Low | QC-Mid | QC-High | QC-Dil |
|---|---|---|---|---|
| Analytical Run | 1.50 | 8.00 | 80.0 | 100 |
| B01 | 1.46 | 8.55 | 88.0 | NA |
| | 1.40 | 8.03 | 79.3 | NA |
| | 1.47 | 8.53 | 88.4 | NA |
| B02 | 1.53 | 8.43 | 84.4 | NA |
| | 1.57 | 8.63 | 86.1 | NA |
| | 1.48 | 7.93 | 79.8 | NA |
| B03 | 1.55 | 7.79 | 75.5 | NA |
| | 1.44 | 7.56 | 79.7 | NA |
| | 1.49 | 7.78 | 78.1 | NA |
| B04 | 1.58 | 8.15 | 82.2 | 102 |
| | 1.52 | 8.17 | 68.7 | 110 |
| | 1.69 | 8.08 | 82.7 | 96.4 |
| Mean | 1.52 | 8.14 | 81.1 | 103 |
| SD | 0.0769 | 0.343 | 5.57 | 6.84 |
| % CV | 5.1 | 4.2 | 6.9 | 6.6 |
| % Bias | 1.0 | 1.7 | 1.3 | 2.8 |
| n | 12 | 12 | 12 | 3 |

TABLE 16
Glycodeoxycholic Acid: Concentrations in Human Saliva Samples

| | Concentration (ng/mL) | | | | |
|---|---|---|---|---|---|
| | Time Point (hour post breakfast) | | | | |
| Subject No. | Pre | 1 | 2 | 3 | 4 |
| 001-0001 | BQL | 0.688 | 0.998 | 1.02 | 0.640 |
| 001-0002 | BQL | BQL | BQL | BQL | BQL |
| 001-0003 | 1.19 | 0.580 | 0.582 | 0.565 | 0.622 |
| 001-0004 | BQL | BQL | BQL | BQL | BQL |
| 001-0005 | BQL | BQL | BQL | BQL | BQL |
| 001-0006 | BQL | BQL | BQL | BQL | BQL |
| 001-0007 | BQL | 0.747 | 1.08 | BQL | BQL |
| 001-0008 | 3.69 | 1.94 | 2.70 | 2.08 | 2.46 |
| 001-0009 | 1.81 | 1.64 | 4.55 | 5.15 | 6.57 |
| 001-0010 | BQL | BQL | BQL | BQL | BQL |
| 001-0011 | 1.25 | 0.542 | 0.546 | BQL | BQL |
| 001-0012 | BQL | 0.616 | 0.761 | 0.791 | BQL |
| 001-0013 | BQL | BQL | BQL | BQL | BQL |
| 001-0014 | 1.06 | 0.700 | 1.59 | 1.70 | 1.36 |
| 001-0015 | BQL | BQL | 0.651 | BQL | BQL |
| 001-0016 | 1.11 | BQL | BQL | BQL | BQL |
| 001-0017 | BQL | 1.04 | BQL | BQL | BQL |
| 001-0018 | BQL | BQL | BQL | BQL | BQL |
| 001-0019 | BQL | BQL | BQL | 0.848 | 0.747 |
| 001-0020 | 0.715 | 0.877 | 1.81 | 0.864 | 0.529 |
| 001-0021 | BQL | BQL | BQL | BQL | BQL |
| 001-0022 | 0.960 | 0.889 | 1.45 | 0.995 | 0.802 |
| 001-0023 | BQL | BQL | BQL | BQL | BQL |
| 001-0024 | 1.73 | 0.725 | 0.603 | BQL | 0.567 |
| 001-0025 | BQL | BQL | BQL | BQL | BQL |
| 001-0026 | 0.911 | 0.531 | 0.520 | BQL | BQL |
| 001-0027 | BQL | BQL | BQL | BQL | BQL |
| 001-0028 | 1.44 | 0.545 | 0.581 | 0.757 | 0.531 |
| 001-0029 | 0.734 | 0.686 | 1.15 | 0.673 | 0.531 |
| 001-0030 | BQL | BQL | BQL | BQL | BQL |
| 001-0031 | BQL | BQL | BQL | BQL | BQL |
| 001-0032 | 0.888 | 0.538 | 2.00 | 1.31 | 1.17 |
| 001-0033 | BQL | BQL | BQL | BQL | BQL |
| 001-0034 | 2.10 | 1.33 | 1.29 | 1.07 | 1.31 |
| 001-0035 | BQL | BQL | BQL | BQL | BQL |
| 001-0036 | 10.1 | 11.2 | 21.6 | 17.1 | 8.97 |
| 001-0037 | BQL | BQL | BQL | BQL | BQL |
| 001-0038 | 1.41 | 0.514 | 1.30 | 0.786 | 0.556 |
| 001-0039 | 0.628 | BQL | 0.629 | 0.731 | 0.547 |
| 001-0040 | 2.71 | 1.09 | 0.947 | 0.840 | 0.541 |
| 001-0041 | BQL | BQL | BQL | BQL | BQL |
| 001-0042 | BQL | BQL | BQL | BQL | BQL |
| 001-0043 | BQL | BQL | BQL | BQL | BQL |
| 001-0044 | 3.66 | 2.81 | 3.89 | 3.87 | 2.72 |
| 001-0045 | BQL | BQL | 0.663 | 0.574 | BQL |
| 001-0046 | 1.00 | BQL | BQL | 0.853 | 1.54 |
| 001-0047 | 0.501 | BQL | BQL | BQL | BQL |
| 001-0048 | BQL | BQL | BQL | BQL | BQL |
| 001-0049 | BQL | BQL | 0.510 | 0.612 | 0.511 |
| 001-0050 | 0.767 | 0.758 | 0.785 | 0.827 | 0.848 |
| 001-0051 | BQL | BQL | BQL | BQL | BQL |
| 001-0052 | BQL | BQL | BQL | BQL | BQL |
| 001-0053 | 1.20 | BQL | BQL | BQL | BQL |
| 001-0054 | BQL | BQL | BQL | BQL | BQL |
| 001-0055 | BQL | 0.873 | 0.979 | 0.706 | 0.774 |
| 001-0056 | 0.680 | 0.658 | 0.666 | BQL | 0.524 |
| 001-0057 | BQL | BQL | 0.733 | BQL | BQL |
| 001-0058 | 2.36 | 0.748 | 0.854 | 0.606 | BQL |
| 001-0059 | BQL | BQL | BQL | BQL | BQL |
| 001-0060 | 1.75 | 4.13 | 3.34 | 1.20 | 3.65 |

BQL: Below quantitation limit (0.500 ng/mL)

TABLE 17
Glycodeoxycholic Acid: Calibration Standard Back-Calculated Concentrations in
Cholestyramine-Treated Human Saliva

| | Concentration (ng/mL) | | | | | | | | |
|---|---|---|---|---|---|---|---|---|---|
| Analytical | Std-1 | Std-2 | Std-3 | Std-4 | Std-5 | Std-6 | Std-7 | Std-8 | Std-9 |
| Run | 0.500 | 0.750 | 1.00 | 2.00 | 5.00 | 10.0 | 50.0 | 75.0 | 100 |
| B01 | 0.468 | 0.704 | 1.04 | 2.13 | 4.96 | 9.28 | 38.4 | 76.9 | 98.7 |
| | 0.516 | 0.711 | 1.07 | 2.37 | 5.09 | 9.84 | NR | 77.8 | 106 |
| B02 | 0.511 | 0.780 | 0.956 | 1.95 | 4.96 | 9.55 | 50.2 | 73.9 | 107 |
| | 0.498 | 0.759 | 0.948 | 1.99 | 5.27 | 10.0 | 51.0 | 76.9 | 95.1 |
| B03 | 0.513 | 0.727 | 0.988 | 2.15 | 5.18 | 9.49 | 47.6 | 76.2 | 99.8 |
| | 0.499 | 0.740 | 0.984 | 1.97 | 5.16 | 10.0 | 48.7 | 71.9 | 107 |
| Mean | 0.501 | 0.737 | 0.998 | 2.09 | 5.10 | 9.69 | 47.2 | 75.6 | 102 |
| SD | 0.0177 | 0.0290 | 0.0480 | 0.160 | 0.125 | 0.297 | 5.08 | 2.24 | 5.08 |
| % CV | 3.5 | 3.9 | 4.8 | 7.6 | 2.4 | 3.1 | 10.8 | 3.0 | 5.0 |
| % Bias | 0.2 | −1.8 | −0.2 | 4.7 | 2.1 | −3.1 | −5.6 | 0.8 | 2.3 |
| n | 6 | 6 | 6 | 6 | 6 | 6 | 5 | 6 | 6 |

NR: Not reported

TABLE 18
Glycodeoxycholic Acid: Batch Acceptance Quality
Controls (Cholestyramine-Treated Human Saliva)

| | Concentration (ng/mL) | | |
|---|---|---|---|
| | QC-Low | QC-Mid | QC-High |
| Analytical Run | 1.50 | 8.00 | 80.0 |
| B01 | 1.55 | 8.09 | 82.1 |
| | 1.54 | 8.60 | 84.9 |
| | 1.56 | 8.38 | 87.6 |
| B02 | 1.55 | 7.65 | 83.8 |
| | 1.61 | 8.10 | 80.6 |
| | 1.68 | 8.23 | 82.1 |
| B03 | 1.52 | 7.68 | 82.0 |
| | 1.53 | 7.53 | 79.9 |
| | 1.50 | 7.35 | 77.5 |
| Mean | 1.56 | 7.96 | 82.3 |
| SD | 0.0543 | 0.422 | 2.94 |
| % CV | 3.5 | 5.3 | 3.6 |
| % Bias | 4.0 | −0.5 | 2.8 |
| n | 9 | 9 | 9 |

TABLE 19
Lithocholic Acid: Concentrations in Human Saliva Samples

| | Concentration (ng/mL) | | | | |
|---|---|---|---|---|---|
| | Time Point (hour post breakfast) | | | | |
| Subject No. | Pre | 1 | 2 | 3 | 4 |
| 001-0001 | BQL | BQL | BQL | BQL | BQL |
| 001-0002 | BQL | BQL | BQL | BQL | BQL |
| 001-0003 | BQL | BQL | BQL | BQL | BQL |
| 001-0004 | BQL | BQL | BQL | BQL | BQL |
| 001-0005 | BQL | BQL | BQL | BQL | BQL |
| 001-0006 | BQL | BQL | BQL | BQL | BQL |
| 001-0007 | BQL | BQL | BQL | BQL | BQL |
| 001-0008 | BQL | BQL | BQL | BQL | BQL |
| 001-0009 | BQL | BQL | BQL | BQL | BQL |
| 001-0010 | BQL | BQL | BQL | BQL | BQL |
| 001-0011 | BQL | BQL | BQL | BQL | BQL |
| 001-0012 | BQL | BQL | BQL | BQL | BQL |
| 001-0013 | BQL | BQL | BQL | BQL | BQL |
| 001-0014 | BQL | BQL | BQL | BQL | BQL |
| 001-0015 | BQL | BQL | BQL | BQL | BQL |
| 001-0016 | BQL | BQL | BQL | BQL | BQL |
| 001-0017 | BQL | BQL | BQL | BQL | BQL |
| 001-0018 | BQL | BQL | BQL | BQL | BQL |
| 001-0019 | BQL | BQL | BQL | BQL | BQL |
| 001-0020 | BQL | BQL | BQL | BQL | BQL |
| 001-0021 | BQL | BQL | BQL | BQL | BQL |
| 001-0022 | BQL | BQL | BQL | BQL | BQL |
| 001-0023 | BQL | BQL | BQL | BQL | BQL |
| 001-0024 | BQL | BQL | BQL | BQL | BQL |
| 001-0025 | BQL | BQL | BQL | BQL | BQL |
| 001-0026 | BQL | BQL | BQL | BQL | BQL |
| 001-0027 | BQL | BQL | BQL | BQL | BQL |
| 001-0028 | BQL | BQL | BQL | BQL | BQL |
| 001-0029 | BQL | BQL | BQL | BQL | BQL |
| 001-0030 | BQL | BQL | BQL | BQL | BQL |
| 001-0031 | BQL | BQL | BQL | BQL | BQL |
| 001-0032 | BQL | BQL | BQL | BQL | BQL |
| 001-0033 | BQL | BQL | BQL | BQL | BQL |
| 001-0034 | BQL | BQL | BQL | BQL | BQL |
| 001-0035 | BQL | BQL | BQL | BQL | BQL |
| 001-0036 | BQL | BQL | BQL | BQL | BQL |
| 001-0037 | BQL | BQL | BQL | BQL | BQL |
| 001-0038 | BQL | BQL | BQL | BQL | BQL |
| 001-0039 | BQL | BQL | BQL | BQL | BQL |
| 001-0040 | BQL | BQL | BQL | BQL | BQL |
| 001-0041 | BQL | BQL | BQL | BQL | BQL |
| 001-0042 | BQL | BQL | BQL | BQL | BQL |
| 001-0043 | BQL | BQL | BQL | BQL | BQL |
| 001-0044 | BQL | BQL | BQL | BQL | BQL |
| 001-0045 | BQL | BQL | BQL | BQL | BQL |
| 001-0046 | BQL | BQL | BQL | BQL | BQL |
| 001-0047 | BQL | BQL | BQL | BQL | BQL |
| 001-0048 | BQL | BQL | BQL | BQL | BQL |
| 001-0049 | BQL | BQL | BQL | BQL | BQL |
| 001-0050 | BQL | BQL | BQL | BQL | BQL |
| 001-0051 | BQL | BQL | BQL | BQL | BQL |
| 001-0052 | BQL | BQL | BQL | BQL | BQL |
| 001-0053 | BQL | BQL | BQL | BQL | BQL |
| 001-0054 | BQL | BQL | BQL | BQL | BQL |
| 001-0055 | BQL | BQL | BQL | BQL | BQL |
| 001-0056 | BQL | BQL | BQL | BQL | BQL |
| 001-0057 | BQL | BQL | BQL | BQL | BQL |
| 001-0058 | BQL | BQL | BQL | BQL | BQL |
| 001-0059 | BQL | BQL | BQL | BQL | BQL |
| 001-0060 | BQL | BQL | BQL | BQL | BQL |

BQL: Below quantitation limit (10.0 ng/mL)

TABLE 20
Lithocholic Acid: Calibration Standard Back-Calculated
Concentrations in Cholestyramine-Treated Human Saliva

| | Concentration (ng/mL) | | | | | | |
|---|---|---|---|---|---|---|---|
| | Std-1 | Std-2 | Std-3 | Std-4 | Std-5 | Std-6 | Std-7 |
| Analytical Run | 10.0 | 20.0 | 50.0 | 100 | 500 | 750 | 1000 |
| B01 | 10.3 | 20.1 | 51.5 | 80.0 | NR | 763 | 1060 |
| | 8.66 | 24.8 | 47.0 | 106 | NR | 756 | 951 |
| B02 | 9.73 | 22.2 | 53.4 | 90.1 | 534 | 922 | 1030 |
| | 10.4 | 17.6 | 47.0 | 96.4 | 467 | 685 | 956 |
| B03 | 10.8 | 19.8 | 46.4 | 83.9 | 445 | 879 | 1070 |
| | 9.89 | 17.6 | 58.7 | 92.0 | 525 | 699 | 1090 |
| Mean | 9.96 | 20.4 | 50.7 | 91.4 | 493 | 784 | 1030 |
| SD | 0.743 | 2.78 | 4.85 | 9.23 | 43.5 | 96.2 | 59.5 |
| % CV | 7.5 | 13.7 | 9.6 | 10.1 | 8.8 | 12.3 | 5.8 |
| % Bias | −0.4 | 1.8 | 1.3 | −8.6 | −1.5 | 4.5 | 2.6 |
| n | 6 | 6 | 6 | 6 | 4 | 6 | 6 |

NR: Not reported

TABLE 21
Lithocholic Acid: Batch Acceptance Quality Controls
(Cholestyramine-Treated Human Saliva)

| | Concentration (ng/mL) | | |
|---|---|---|---|
| | QC-Low | QC-Mid | QC-High |
| Analytical Run | 15.0 | 80.0 | 800 |
| B01 | 15.3 | 81.4 | 834 |
| | 14.1 | 93.2 | 975 |
| | 15.4 | 100 | 953 |
| B02 | 15.5 | 76.4 | 802 |
| | 16.1 | 87.8 | 810 |
| | 17.3 | 74.9 | 727 |
| B03 | 13.4 | 80.0 | 781 |
| | 13.6 | 72.5 | 783 |
| | 12.6 | 68.1 | 669 |
| Mean | 14.8 | 81.6 | 815 |
| SD | 1.49 | 10.3 | 97.7 |
| % CV | 10.1 | 12.6 | 12.0 |
| % Bias | −1.3 | 2.0 | 1.9 |
| n | 9 | 9 | 9 |

TABLE 22
Taurodeoxycholic Acid: Concentrations in Human Saliva Samples

| | Concentration (ng/mL) | | | | |
|---|---|---|---|---|---|
| | Time Point (hour post breakfast) | | | | |
| Subject No. | Pre | 1 | 2 | 3 | 4 |
| 001-0001 | BQL | BQL | BQL | BQL | BQL |
| 001-0002 | BQL | BQL | BQL | BQL | BQL |
| 001-0003 | BQL | BQL | BQL | BQL | BQL |
| 001-0004 | BQL | BQL | BQL | BQL | BQL |
| 001-0005 | BQL | BQL | BQL | BQL | BQL |
| 001-0006 | BQL | BQL | BQL | BQL | BQL |
| 001-0007 | BQL | BQL | BQL | BQL | BQL |
| 001-0008 | 0.614 | BQL | BQL | BQL | BQL |
| 001-0009 | 0.696 | BQL | 1.41 | 1.72 | 2.34 |
| 001-0010 | BQL | BQL | BQL | BQL | BQL |
| 001-0011 | BQL | BQL | BQL | BQL | BQL |
| 001-0012 | BQL | BQL | BQL | BQL | BQL |
| 001-0013 | BQL | BQL | BQL | BQL | BQL |
| 001-0014 | BQL | BQL | BQL | BQL | BQL |
| 001-0015 | BQL | BQL | BQL | BQL | BQL |
| 001-0016 | BQL | BQL | BQL | BQL | BQL |
| 001-0017 | BQL | BQL | BQL | BQL | BQL |
| 001-0018 | BQL | BQL | BQL | BQL | BQL |
| 001-0019 | BQL | BQL | BQL | BQL | BQL |
| 001-0020 | BQL | BQL | BQL | BQL | BQL |
| 001-0021 | BQL | BQL | BQL | BQL | BQL |
| 001-0022 | BQL | BQL | BQL | BQL | BQL |
| 001-0023 | BQL | BQL | BQL | BQL | BQL |
| 001-0024 | 0.777 | BQL | BQL | BQL | BQL |
| 001-0025 | BQL | BQL | BQL | BQL | BQL |
| 001-0026 | BQL | BQL | BQL | BQL | BQL |
| 001-0027 | BQL | BQL | BQL | BQL | BQL |
| 001-0028 | BQL | BQL | BQL | BQL | BQL |
| 001-0029 | BQL | BQL | BQL | BQL | BQL |
| 001-0030 | BQL | BQL | BQL | BQL | BQL |
| 001-0031 | BQL | BQL | BQL | BQL | BQL |
| 001-0032 | BQL | BQL | BQL | BQL | BQL |
| 001-0033 | BQL | BQL | BQL | BQL | BQL |
| 001-0034 | BQL | BQL | BQL | BQL | BQL |
| 001-0035 | BQL | BQL | BQL | BQL | BQL |
| 001-0036 | 1.89 | 2.06 | 4.36 | 3.22 | 1.42 |
| 001-0037 | BQL | BQL | BQL | BQL | BQL |
| 001-0038 | BQL | BQL | BQL | BQL | BQL |
| 001-0039 | BQL | BQL | BQL | BQL | BQL |
| 001-0040 | BQL | BQL | BQL | BQL | BQL |
| 001-0041 | BQL | BQL | BQL | BQL | BQL |
| 001-0042 | BQL | BQL | BQL | BQL | BQL |
| 001-0043 | BQL | BQL | BQL | BQL | BQL |
| 001-0044 | 0.744 | BQL | BQL | 0.509 | BQL |
| 001-0045 | BQL | BQL | BQL | BQL | BQL |
| 001-0046 | BQL | BQL | BQL | BQL | BQL |
| 001-0047 | BQL | BQL | BQL | BQL | BQL |
| 001-0048 | BQL | BQL | BQL | BQL | BQL |
| 001-0049 | BQL | BQL | BQL | BQL | BQL |
| 001-0050 | BQL | BQL | BQL | BQL | BQL |
| 001-0051 | BQL | BQL | BQL | BQL | BQL |
| 001-0052 | BQL | BQL | BQL | BQL | BQL |
| 001-0053 | BQL | BQL | BQL | BQL | BQL |
| 001-0054 | BQL | BQL | BQL | BQL | BQL |
| 001-0055 | 0.976 | BQL | BQL | BQL | BQL |
| 001-0056 | BQL | BQL | BQL | BQL | BQL |
| 001-0057 | BQL | BQL | 0.627 | BQL | BQL |
| 001-0058 | BQL | BQL | BQL | BQL | BQL |
| 001-0059 | BQL | BQL | BQL | BQL | BQL |
| 001-0060 | BQL | BQL | BQL | BQL | BQL |

BQL: Below quantitation limit (0.500 ng/mL)

TABLE 23
Taurodeoxycholic Acid: Calibration Standard Back-Calculated
Concentrations in Cholestyramin-Treated Human Saliva

| | Concentration (ng/mL) | | | | | | | | |
|---|---|---|---|---|---|---|---|---|---|
| Analytical | Std-1 | Std-2 | Std-3 | Std-4 | Std-5 | Std-6 | Std-7 | Std-8 | Std-9 |
| Run | 0.500 | 0.750 | 1.00 | 2.00 | 5.00 | 10.0 | 50.0 | 75.0 | 100 |
| B01 | 0.496 | 0.716 | 1.08 | 2.10 | 5.13 | 9.33 | 36.9 | 77.1 | 102 |
| | 0.471 | 0.743 | 1.03 | 2.32 | 4.94 | 9.74 | NR | 78.5 | 105 |
| B02 | 0.497 | 0.735 | 0.944 | 2.00 | 4.85 | 9.41 | 49.5 | 74.9 | 108 |
| | 0.518 | 0.770 | 0.992 | 2.00 | 5.19 | 10.1 | 51.8 | 76.1 | 95.7 |
| B03 | 0.509 | 0.737 | 0.989 | 2.11 | 5.04 | 9.53 | 49.5 | 77.4 | 99.0 |
| | 0.511 | 0.738 | 0.950 | 1.99 | 5.17 | 9.80 | 48.4 | 72.1 | 109 |
| Mean | 0.500 | 0.740 | 0.998 | 2.09 | 5.05 | 9.65 | 47.2 | 76.0 | 103 |
| SD | 0.0167 | 0.0175 | 0.0512 | 0.126 | 0.136 | 0.285 | 5.90 | 2.27 | 5.20 |
| % CV | 3.3 | 2.4 | 5.1 | 6.0 | 2.7 | 3.0 | 12.5 | 3.0 | 5.0 |
| % Bias | 0.1 | −1.4 | −0.2 | 4.3 | 1.1 | −3.5 | −5.6 | 1.4 | 3.1 |
| n | 6 | 6 | 6 | 6 | 6 | 6 | 5 | 6 | 6 |

TABLE 24
Taurodeoxycholic Acid: Batch Acceptance Quality
Controls (Cholestyramine-Treated Human Saliva)

| | Concentration (ng/mL) | | |
|---|---|---|---|
| | QC-Low | QC-Mid | QC-High |
| Analytical Run | 1.50 | 8.00 | 80.0 |
| B01 | 1.55 | 8.35 | 86.3 |
| | 1.55 | 8.74 | 89.4 |
| | 1.63 | 8.71 | 89.2 |
| B02 | 1.58 | 7.87 | 85.7 |
| | 1.55 | 8.18 | 83.3 |
| | 1.67 | 8.18 | 85.6 |
| B03 | 1.56 | 8.07 | 86.2 |
| | 1.57 | 7.66 | 82.7 |
| | 1.45 | 7.33 | 80.7 |
| Mean | 1.57 | 8.12 | 85.5 |
| SD | 0.0606 | 0.460 | 2.87 |
| % CV | 3.9 | 5.7 | 3.4 |
| % Bias | 4.5 | 1.5 | 6.8 |
| n | 9 | 9 | 9 |

TABLE 25
Taurocholic Acid: Concentrations in Human Saliva Samples

| | Concentration (ng/mL) | | | | |
|---|---|---|---|---|---|
| | Time Point (hour post breakfast) | | | | |
| Subject No. | Pre | 1 | 2 | 3 | 4 |
| 001-0001 | BQL | BQL | BQL | BQL | BQL |
| 001-0002 | BQL | BQL | BQL | BQL | BQL |
| 001-0003 | BQL | BQL | BQL | BQL | BQL |
| 001-0004 | BQL | BQL | BQL | BQL | BQL |
| 001-0005 | BQL | BQL | BQL | BQL | BQL |
| 001-0006 | BQL | 0.502 | BQL | BQL | BQL |
| 001-0007 | BQL | BQL | BQL | BQL | BQL |
| 001-0008 | 0.543 | BQL | BQL | 0.671 | 0.616 |
| 001-0009 | BQL | BQL | 2.56 | 1.93 | 2.14 |
| 001-0010 | BQL | BQL | BQL | BQL | BQL |
| 001-0011 | BQL | BQL | BQL | BQL | BQL |
| 001-0012 | BQL | BQL | 1.25 | 0.811 | 0.535 |
| 001-0013 | BQL | BQL | BQL | BQL | BQL |
| 001-0014 | BQL | BQL | BQL | BQL | BQL |
| 001-0015 | BQL | BQL | BQL | BQL | BQL |
| 001-0016 | BQL | BQL | BQL | BQL | BQL |
| 001-0017 | BQL | BQL | BQL | BQL | BQL |
| 001-0018 | BQL | BQL | BQL | BQL | BQL |
| 001-0019 | BQL | BQL | BQL | BQL | BQL |
| 001-0020 | BQL | BQL | BQL | BQL | BQL |
| 001-0021 | BQL | BQL | BQL | BQL | BQL |
| 001-0022 | BQL | 0.656 | BQL | BQL | BQL |
| 001-0023 | BQL | BQL | BQL | BQL | BQL |
| 001-0024 | BQL | 0.947 | BQL | BQL | BQL |
| 001-0025 | BQL | BQL | BQL | BQL | BQL |
| 001-0026 | BQL | 1.41 | 0.834 | BQL | BQL |
| 001-0027 | BQL | BQL | BQL | BQL | BQL |
| 001-0028 | BQL | BQL | BQL | BQL | BQL |
| 001-0029 | BQL | BQL | BQL | BQL | BQL |
| 001-0030 | BQL | BQL | BQL | BQL | BQL |
| 001-0031 | BQL | BQL | BQL | BQL | BQL |
| 001-0032 | BQL | BQL | BQL | BQL | BQL |
| 001-0033 | BQL | 1.00 | BQL | BQL | BQL |
| 001-0034 | BQL | 1.69 | 0.581 | BQL | BQL |
| 001-0035 | BQL | BQL | BQL | BQL | BQL |
| 001-0036 | 0.939 | 1.72 | 3.25 | 2.40 | 0.994 |
| 001-0037 | BQL | BQL | BQL | BQL | BQL |
| 001-0038 | BQL | BQL | BQL | BQL | BQL |
| 001-0039 | BQL | BQL | BQL | BQL | BQL |
| 001-0040 | BQL | 2.62 | 1.63 | 1.15 | 0.898 |
| 001-0041 | BQL | BQL | BQL | BQL | BQL |
| 001-0042 | BQL | BQL | BQL | BQL | BQL |
| 001-0043 | BQL | 0.609 | 0.542 | BQL | BQL |
| 001-0044 | BQL | 2.05 | 1.86 | 1.51 | 0.860 |
| 001-0045 | BQL | 2.48 | 1.50 | 0.656 | BQL |
| 001-0046 | BQL | 1.25 | BQL | BQL | BQL |
| 001-0047 | BQL | 1.90 | 0.832 | 0.844 | BQL |
| 001-0048 | BQL | 1.52 | 0.652 | BQL | BQL |
| 001-0049 | BQL | BQL | BQL | BQL | BQL |
| 001-0050 | 0.847 | 1.61 | 1.08 | 0.944 | 0.564 |
| 001-0051 | BQL | 0.502 | BQL | BQL | BQL |
| 001-0052 | BQL | BQL | BQL | BQL | BQL |
| 001-0053 | BQL | 1.85 | 1.41 | 0.852 | BQL |
| 001-0054 | BQL | 0.549 | 0.869 | 0.549 | BQL |
| 001-0055 | 0.835 | 2.19 | 0.996 | 0.595 | 0.531 |
| 001-0056 | BQL | 0.721 | BQL | BQL | BQL |
| 001-0057 | 0.772 | 2.73 | 3.68 | 1.79 | 2.09 |
| 001-0058 | BQL | 1.10 | BQL | BQL | BQL |
| 001-0059 | BQL | 4.09 | BQL | BQL | BQL |
| 001-0060 | BQL | 0.635 | 0.547 | BQL | BQL |

BQL: Below quantitation limit (0.500 ng/mL)

TABLE 26
Taurocholic Acid: Calibration Standard Back-Calculated Concentrations in
Cholestyramine-Treated Human Saliva

| | Concentration (ng/mL) | | | | | | | | |
|---|---|---|---|---|---|---|---|---|---|
| Analytical | Std-1 | Std-2 | Std-3 | Std-4 | Std-5 | Std-6 | Std-7 | Std-8 | Std-9 |
| Run | 0.500 | 0.750 | 1.00 | 2.00 | 5.00 | 10.0 | 50.0 | 75.0 | 100 |
| B01 | 0.479 | 0.736 | 1.04 | 2.03 | 4.59 | 8.92 | 43.6 | 74.4 | 97.0 |
| | 0.505 | 0.735 | 1.03 | 2.26 | 4.96 | 9.43 | 62.1 | 75.6 | 102 |
| B02 | 0.501 | 0.703 | 0.938 | 1.95 | 4.89 | 9.44 | 49.9 | 75.9 | 101 |
| | 0.515 | 0.778 | 1.04 | 2.03 | 5.23 | 9.99 | 51.0 | 77.2 | 98.8 |
| B03 | 0.530 | 0.708 | 0.937 | 2.08 | 5.22 | 9.66 | 49.2 | 77.2 | 101 |
| | 0.504 | 0.767 | 0.94 | 1.99 | 5.21 | 9.8 | 49.6 | 71.5 | 105 |
| Mean | 0.506 | 0.738 | 0.988 | 2.06 | 5.02 | 9.55 | 50.9 | 75.3 | 101 |
| SD | 0.0168 | 0.0303 | 0.0536 | 0.109 | 0.255 | 0.378 | 6.07 | 2.14 | 2.74 |
| % CV | 3.3 | 4.1 | 5.4 | 5.3 | 5.1 | 4.0 | 11.9 | 2.8 | 2.7 |
| % Bias | 1.1 | −1.6 | −1.2 | 2.8 | 0.3 | −4.5 | 1.8 | 0.4 | 0.8 |
| n | 6 | 6 | 6 | 6 | 6 | 6 | 6 | 6 | 6 |

TABLE 27
Taurocholic Acid: Batch Acceptance Quality Controls
(Cholestyramine-Treated Human Saliva)

| | Concentration (ng/mL) | | |
|---|---|---|---|
| | QC-Low | QC-Mid | QC-High |
| Analytical Run | 1.50 | 8.00 | 80.0 |
| B01 | 1.48 | 8.04 | 81.8 |
| | 1.54 | 8.11 | 88.1 |
| | 1.65 | 8.43 | 87.0 |
| B02 | 1.59 | 7.98 | 85.4 |
| | 1.61 | 8.24 | 85.3 |
| | 1.64 | 8.44 | 84.9 |
| B03 | 1.55 | 8.45 | 88.0 |
| | 1.53 | 8.14 | 86.5 |
| | 1.54 | 7.82 | 85.8 |
| Mean | 1.57 | 8.18 | 85.9 |
| SD | 0.0561 | 0.224 | 1.91 |
| % CV | 3.6 | 2.7 | 2.2 |
| % Bias | 4.7 | 2.3 | 7.3 |
| n | 9 | 9 | 9 |

TABLE 28
Glycochenodeoxycholic Acid: Concentrations
in Human Saliva Samples

| | Concentration (ng/mL) | | | | |
|---|---|---|---|---|---|
| | Time Point (hour post breakfast) | | | | |
| Subject No. | Pre | 1 | 2 | 3 | 4 |
| 001-0001 | BQL | 0.670 | 1.02 | 1.18 | 0.797 |
| 001-0002 | 0.550 | BQL | BQL | BQL | BQL |
| 001-0003 | 2.16 | 1.27 | 1.40 | 1.29 | 1.42 |
| 001-0004 | 1.77 | 0.998 | 1.32 | 1.46 | 1.84 |
| 001-0005 | BQL | BQL | BQL | BQL | BQL |
| 001-0006 | 1.04 | BQL | BQL | 0.528 | BQL |
| 001-0007 | 0.774 | 1.57 | 2.67 | 0.762 | 0.776 |
| 001-0008 | 6.29 | 3.51 | 4.99 | 3.82 | 4.56 |
| 001-0009 | 1.24 | 0.990 | 3.45 | 3.80 | 4.87 |
| 001-0010 | 1.06 | 0.756 | 0.777 | 0.739 | 0.913 |
| 001-0011 | 2.06 | 1.07 | 0.892 | 0.690 | 0.784 |
| 001-0012 | 0.713 | 1.92 | 2.46 | 2.57 | 1.64 |
| 001-0013 | 0.798 | BQL | BQL | BQL | BQL |
| 001-0014 | 1.07 | 0.663 | 1.53 | 1.75 | 1.46 |
| 001-0015 | 0.582 | 0.903 | 1.41 | 0.570 | 0.753 |
| 001-0016 | 3.88 | 1.64 | 1.79 | 1.05 | 1.67 |
| 001-0017 | BQL | 1.40 | 0.500 | BQL | BQL |
| 001-0018 | 1.01 | 1.12 | 1.10 | 0.976 | 1.16 |
| 001-0019 | 2.13 | 1.98 | 2.79 | 4.76 | 4.01 |
| 001-0020 | 1.18 | 1.25 | 3.24 | 1.63 | 1.02 |
| 001-0021 | BQL | 0.576 | 0.804 | 0.878 | 1.03 |
| 001-0022 | 1.43 | 1.26 | 2.08 | 1.57 | 1.22 |
| 001-0023 | BQL | BQL | BQL | BQL | BQL |
| 001-0024 | 8.59 | 0.936 | 0.798 | 0.564 | 0.822 |
| 001-0025 | BQL | BQL | BQL | BQL | BQL |
| 001-0026 | 2.89 | 1.52 | 1.56 | 1.33 | 1.62 |
| 001-0027 | BQL | BQL | BQL | BQL | BQL |
| 001-0028 | 5.89 | 1.81 | 1.93 | 2.34 | 1.80 |
| 001-0029 | BQL | BQL | 0.666 | BQL | BQL |
| 001-0030 | BQL | BQL | BQL | BQL | BQL |
| 001-0031 | BQL | BQL | BQL | BQL | BQL |
| 001-0032 | 1.32 | 0.944 | 3.18 | 2.09 | 1.80 |
| 001-0033 | 1.69 | 1.37 | 2.18 | 1.55 | 1.14 |
| 001-0034 | 2.44 | 1.75 | 1.61 | 1.45 | 1.93 |
| 001-0035 | 2.09 | 0.759 | 0.671 | 0.601 | 0.668 |
| 001-0036 | 12.3 | 14.8 | 29.2 | 24.9 | 12.9 |
| 001-0037 | BQL | BQL | BQL | BQL | 0.505 |
| 001-0038 | 4.77 | 1.61 | 4.07 | 2.69 | 1.87 |
| 001-0039 | 0.812 | BQL | 0.825 | 0.933 | 0.673 |
| 001-0021 | BQL | 0.576 | 0.804 | 0.878 | 1.03 |
| 001-0041 | BQL | BQL | BQL | 0.552 | BQL |
| 001-0042 | 1.22 | BQL | BQL | BQL | BQL |
| 001-0043 | 0.591 | 0.511 | 0.753 | 0.557 | BQL |
| 001-0044 | 2.52 | 1.81 | 2.77 | 2.82 | 1.91 |
| 001-0045 | 1.24 | 2.39 | 3.25 | 2.81 | 2.61 |
| 001-0046 | 2.15 | BQL | 1.09 | 2.35 | 3.19 |
| 001-0047 | 2.03 | 1.35 | 1.37 | 1.46 | 1.11 |
| 001-0048 | 0.698 | 0.728 | 1.43 | 1.48 | 1.41 |
| 001-0049 | 0.546 | 0.942 | 1.54 | 1.84 | 1.51 |
| 001-0050 | 0.869 | 0.993 | 1.05 | 1.18 | 1.39 |
| 001-0051 | BQL | 0.525 | 1.31 | 1.06 | 1.05 |
| 001-0052 | 0.548 | 1.01 | 1.21 | 1.27 | 1.10 |
| 001-0053 | 2.68 | 2.03 | 2.48 | 2.13 | 1.08 |
| 001-0054 | BQL | BQL | 0.689 | 0.728 | 0.553 |
| 001-0055 | 1.85 | 0.752 | 0.901 | 0.597 | 0.767 |
| 001-0056 | 1.51 | 1.18 | 1.16 | 0.938 | 1.04 |
| 001-0057 | 1.60 | 2.53 | 6.12 | 2.06 | 2.86 |
| 001-0058 | 5.89 | 1.88 | 2.28 | 1.58 | 0.910 |
| 001-0059 | 0.697 | 0.518 | BQL | BQL | BQL |
| 001-0060 | 11.7 | 29.9 | 24.5 | 8.80 | 28.5 |

BQL: Below quantitation limit (0.500 ng/mL)

TABLE 29
Glycochenodeoxycholic Acid: Calibration Standard Back-Calculated Concentrations in
Cholestyramine-Treated Human Saliva

| | Concentration (ng/mL) | | | | | | | | |
|---|---|---|---|---|---|---|---|---|---|
| Analytical | Std-1 | Std-2 | Std-3 | Std-4 | Std-5 | Std-6 | Std-7 | Std-8 | Std-9 |
| Run | 0.500 | 0.750 | 1.00 | 2.00 | 5.00 | 10.0 | 50.0 | 75.0 | 100 |
| B01 | 0.473 | 0.710 | 1.04 | 2.13 | 4.84 | 9.17 | 38.5 | 77.2 | 101 |
| | 0.502 | 0.742 | 1.03 | 2.38 | 5.15 | 9.84 | NR | 77.9 | 104 |
| B02 | 0.505 | 0.732 | 0.943 | 1.97 | 4.79 | 9.79 | 50.3 | 74.7 | 106 |
| | 0.513 | 0.761 | 1.00 | 2.00 | 5.26 | 9.78 | 51.1 | 76.6 | 96.3 |
| B03 | 0.505 | 0.752 | 0.989 | 2.16 | 5.11 | 9.46 | 49.3 | 75.9 | 98.9 |
| | 0.502 | 0.736 | 0.963 | 1.99 | 5.24 | 9.68 | 49.4 | 71.5 | 106 |
| Mean | 0.500 | 0.739 | 0.994 | 2.11 | 5.07 | 9.62 | 47.7 | 75.6 | 102 |
| SD | 0.0138 | 0.0177 | 0.0375 | 0.156 | 0.202 | 0.259 | 5.21 | 2.30 | 3.98 |
| % CV | 2.8 | 2.4 | 3.8 | 7.4 | 4.0 | 2.7 | 10.9 | 3.0 | 3.9 |
| % Bias | 0.0 | −1.5 | −0.6 | 5.3 | 1.3 | −3.8 | −4.6 | 0.8 | 2.0 |
| n | 6 | 6 | 6 | 6 | 6 | 6 | 5 | 6 | 6 |

TABLE 30
Glycochenodeoxycholic Acid: Batch Acceptance Quality
Controls (Cholestyramine-Treated Human Saliva)

| | Concentration (ng/mL) | | |
|---|---|---|---|
| | QC-Low | QC-Mid | QC-High |
| Analytical Run | 1.50 | 8.00 | 80.0 |
| B01 | 1.63 | 8.74 | 86.8 |
| | 1.63 | 9.10 | 90.6 |
| | 1.71 | 8.90 | 92.5 |
| B02 | 1.70 | 8.20 | 87.6 |
| | 1.68 | 8.60 | 86.4 |
| | 1.78 | 8.62 | 86.9 |
| B03 | 1.62 | 8.18 | 88.0 |
| | 1.62 | 8.10 | 86.6 |
| | 1.58 | 7.77 | 82.6 |
| Mean | 1.66 | 8.47 | 87.6 |
| SD | 0.0615 | 0.430 | 2.78 |
| % CV | 3.7 | 5.1 | 3.2 |
| % Bias | 10.7 | 5.8 | 9.4 |
| n | 9 | 9 | 9 |

TABLE 31
Taurochenodeoxycholic Acid: Concentrations
in Human Saliva Samples

| | Concentration (ng/mL) | | | | |
|---|---|---|---|---|---|
| | Time Point (hour post breakfast) | | | | |
| Subject No. | Pre | 1 | 2 | 3 | 4 |
| 001-0001 | BQL | 2.35 | 1.36 | 1.01 | 0.709 |
| 001-0002 | BQL | 2.18 | BQL | BQL | BQL |
| 001-0003 | 0.818 | 6.74 | 2.44 | 1.37 | 1.07 |
| 001-0004 | BQL | 3.56 | 1.68 | BQL | BQL |
| 001-0005 | BQL | 1.38 | BQL | BQL | BQL |
| 001-0006 | 0.572 | 9.69 | 1.71 | 1.13 | 0.591 |
| 001-0007 | BQL | 1.68 | 1.26 | BQL | BQL |
| 001-0008 | 1.02 | 28.6 | 25.5 | 15.2 | 8.44 |
| 001-0009 | 0.537 | 24.3 | 19.3 | 10.8 | 10.2 |
| 001-0010 | 0.604 | 1.62 | 0.791 | 0.520 | 0.645 |
| 001-0011 | 0.711 | 4.41 | 1.57 | 0.860 | 0.953 |
| 001-0012 | BQL | 18.7 | 14.6 | 9.59 | 4.35 |
| 001-0013 | 0.622 | 1.40 | BQL | BQL | BQL |
| 001-0014 | BQL | 11.9 | 4.72 | 3.86 | 2.16 |
| 001-0015 | BQL | 2.15 | 1.55 | 0.612 | 0.690 |
| 001-0016 | 0.617 | 24.4 | 8.48 | 2.29 | 1.96 |
| 001-0017 | BQL | 0.938 | BQL | BQL | BQL |
| 001-0018 | BQL | 2.70 | 1.20 | 0.683 | BQL |
| 001-0019 | BQL | 3.02 | 3.70 | 3.60 | 1.95 |
| 001-0020 | BQL | 7.68 | 3.35 | 1.18 | 0.651 |
| 001-0021 | BQL | BQL | BQL | BQL | BQL |
| 001-0022 | BQL | 3.52 | 2.13 | 0.883 | 0.546 |
| 001-0023 | BQL | 1.06 | 0.500 | BQL | BQL |
| 001-0024 | 0.942 | 26.0 | 8.36 | 1.44 | 1.34 |
| 001-0025 | BQL | 1.63 | 0.779 | BQL | BQL |
| 001-0026 | 0.891 | 19.6 | 7.30 | 3.16 | 2.59 |
| 001-0027 | 0.503 | BQL | BQL | BQL | BQL |
| 001-0028 | 0.517 | 4.50 | 1.25 | 0.913 | 1.05 |
| 001-0029 | BQL | 6.08 | 1.83 | 0.517 | BQL |
| 001-0030 | BQL | 5.77 | 2.93 | 1.07 | 0.917 |
| 001-0031 | BQL | BQL | BQL | BQL | BQL |
| 001-0032 | BQL | BQL | 0.551 | BQL | BQL |
| 001-0033 | 0.619 | 28.0 | 7.09 | 3.05 | 1.48 |
| 001-0034 | BQL | 44.4 | 14.5 | 6.09 | 4.96 |
| 001-0035 | 0.769 | 2.40 | 0.730 | BQL | BQL |
| 001-0036 | 2.12 | 6.10 | 7.49 | 5.22 | 3.30 |
| 001-0037 | BQL | 3.15 | 1.19 | 0.575 | 0.544 |
| 001-0038 | BQL | 13.1 | 8.74 | 2.45 | 1.22 |
| 001-0039 | BQL | 5.17 | 3.75 | 2.83 | 1.20 |
| 001-0040 | 0.803 | 40.6 | 27.8 | 21.0 | 17.5 |
| 001-0041 | BQL | BQL | BQL | BQL | BQL |
| 001-0042 | BQL | BQL | BQL | BQL | BQL |
| 001-0043 | BQL | 0.609 | 0.542 | BQL | BQL |
| 001-0044 | BQL | 2.05 | 1.86 | 1.51 | 0.860 |
| 001-0045 | BQL | 2.48 | 1.50 | 0.656 | BQL |
| 001-0046 | BQL | 1.25 | BQL | BQL | BQL |
| 001-0047 | BQL | 1.90 | 0.832 | 0.844 | BQL |
| 001-0048 | BQL | 1.52 | 0.652 | BQL | BQL |
| 001-0049 | BQL | BQL | BQL | BQL | BQL |
| 001-0050 | 0.847 | 1.61 | 1.08 | 0.944 | 0.564 |
| 001-0051 | BQL | 0.502 | BQL | BQL | BQL |
| 001-0052 | BQL | BQL | BQL | BQL | BQL |
| 001-0053 | BQL | 1.85 | 1.41 | 0.852 | BQL |
| 001-0054 | BQL | 0.549 | 0.869 | 0.549 | BQL |
| 001-0055 | 0.835 | 2.19 | 0.996 | 0.595 | 0.531 |
| 001-0056 | BQL | 0.721 | BQL | BQL | BQL |
| 001-0057 | 0.772 | 2.73 | 3.68 | 1.79 | 2.09 |
| 001-0058 | BQL | 1.10 | BQL | BQL | BQL |
| 001-0059 | BQL | 4.09 | BQL | BQL | BQL |
| 001-0060 | BQL | 0.635 | 0.547 | BQL | BQL |

BQL: Below quantitation limit (0.500 ng/mL)

TABLE 32
Taurochenodeoxycholic Acid: Calibration Standard Back-Calculated
Concentrations in Cholestyramine-Treated Human Saliva

| | Concentration (ng/mL) | | | | | | | | |
|---|---|---|---|---|---|---|---|---|---|
| Analytical | Std-1 | Std-2 | Std-3 | Std-4 | Std-5 | Std-6 | Std-7 | Std-8 | Std-9 |
| Run | 0.500 | 0.750 | 1.00 | 2.00 | 5.00 | 10.0 | 50.0 | 75.0 | 100 |
| B01 | 0.488 | 0.704 | 1.04 | 2.17 | 5.26 | 9.45 | 35.5 | 79.9 | 104 |
| | 0.492 | 0.738 | 1.03 | 2.33 | 4.87 | 9.65 | NR | 77.8 | 101 |
| B02 | 0.500 | 0.750 | 1.00 | 2.00 | 5.00 | 10.0 | 50.0 | 75.0 | 100 |
| | 0.513 | 0.753 | 1.02 | 2.02 | 5.12 | 10.0 | 51.2 | 77.4 | 96.3 |
| B03 | 0.537 | 0.732 | 0.993 | 2.13 | 5.08 | 9.45 | 48.8 | 77.5 | 99.9 |
| | 0.491 | 0.734 | 0.92 | 1.99 | 5.15 | 9.8 | 48.7 | 72.1 | 111 |
| Mean | 0.504 | 0.735 | 1.00 | 2.11 | 5.08 | 9.72 | 46.8 | 76.6 | 102 |
| SD | 0.0187 | 0.0175 | 0.0425 | 0.132 | 0.134 | 0.248 | 6.42 | 2.71 | 5.04 |
| % CV | 3.7 | 2.4 | 4.2 | 6.3 | 2.6 | 2.6 | 13.7 | 3.5 | 4.9 |
| % Bias | 0.7 | −2.0 | 0.1 | 5.3 | 1.6 | −2.8 | −6.3 | 2.2 | 2.0 |
| n | 6 | 6 | 6 | 6 | 6 | 6 | 5 | 6 | 6 |

NR: Not reported

TABLE 33
Taurochenodeoxycholic Acid: Batch Acceptance Quality
Controls (Cholestyramine-Treated Human Saliva)

| | Concentration (ng/mL) | | |
|---|---|---|---|
| | QC-Low | QC-Mid | QC-High |
| Analytical Run | 1.50 | 8.00 | 80.0 |
| B01 | 1.50 | 8.19 | 83.4 |
| | 1.54 | 8.63 | 85.9 |
| | 1.63 | 8.32 | 85.2 |
| B02 | 1.57 | 7.42 | 81.7 |
| | 1.63 | 7.89 | 80.4 |
| | 1.67 | 7.97 | 83.5 |
| B03 | 1.54 | 7.75 | 82.7 |
| | 1.55 | 7.28 | 81.0 |
| | 1.47 | 7.15 | 77.3 |
| Mean | 1.57 | 7.84 | 82.3 |
| SD | 0.0654 | 0.496 | 2.62 |
| % CV | 4.2 | 6.3 | 3.2 |
| % Bias | 4.4 | −1.9 | 2.9 |
| n | 9 | 9 | 9 |

### Example 3: Qualification of an Lc-Ms/Ms Method for the Analysis of Bile Acids in Human Saliva

Analytes: Cholic acid; Chenodeoxycholic acid; Glycocholic acid; Deoxycholic acid; Glycodeoxycholic acid; Lithocholic acid; Taurodeoxycholic acid; Taurocholic acid; Glycochenodeoxycholic acid; Taurochenodeoxycholic acid.

Matrix: Human saliva; Internal Standard: Glycocholic acid-d4; Matrix for Standards, QCs and Blanks: Cholestyramine-treated human saliva, pooled; Extraction Volume: 100 μL; Extraction Procedure: Protein precipitation; Column: Hypersil Gold, 50×2.1 mm, 1.9 μm; Instrumentation: API-5500; Detection: Electrospray ionization (positive-ion mode); Multiple-reaction-monitoring scan mode; Regression, Weighting: Linear, 1/x2; Acceptance Criteria: Within ±30% from nominal concentrations; Cholic Acid: Assay Performance; Accepted Calibration Range: 0.500 to 100 ng/mL;

Calibration Standard Performance:

| | |
|---|---|
| Inter-Run % Bias | −2.7 → 2.9 |
| Inter-Run % CV | 3.2 → 6.9 |

Quality Control Concentrations: 0.500, 1.50, 8.00 and 80.0 ng/mL

Accuracy and Precision QCs (3 Runs):

| | |
|---|---|
| Intra-Run Accuracy (% bias) | −6.8 → 9.1 |
| Intra-Run Precision (% CV) | 1.1 → 5.9 |
| Inter-Run Accuracy (% bias) | −2.0 → 1.4 |
| Inter-Run Precision (% CV) | 3.9 → 7.4 |

Stability in Treated Human Saliva: 2 Days at −20° C.

Matrix Effects on Quantification: Passed (1 lot of untreated human saliva)

Stability in Untreated Human Saliva: 1 Month at −20° C.; 1 Month at −80° C.

Chenodeoxycholic Acid: Assay Performance

Accepted Calibration Range: 5.00 to 1000 ng/mL

Calibration Standard Performance:

| | |
|---|---|
| Inter-Run % Bias | −13.5 → 13.0 |
| Inter-Run % CV | 4.5 → 9.5 |

Quality Control Concentrations: 5.00, 15.0, 80.0 and 800 ng/mL

Accuracy and Precision QCs (3 Runs):

| | |
|---|---|
| Intra-Run Accuracy (% bias) | −10.3 → 23.7 |
| Intra-Run Precision (% CV) | 3.8 → 13.9 |
| Inter-Run Accuracy (% bias) | −6.0 → 9.3 |
| Inter-Run Precision (% CV) | 8.9 → 15.8 |

Stability in Treated Human Saliva: 2 Days at −20° C.

Matrix Effects on Quantification: Passed (1 lot of untreated human saliva)

Stability in Untreated Human Saliva: 1 Month at −20° C.; 1 Month at −80° C.

Glycocholic Acid: Assay Performance

Accepted Calibration Range: 0.500 to 100 ng/mL

Calibration Standard Performance: Inter-Run % Bias −1.9→3.8; Inter-Run % CV 2.6→6.2

Quality Control Concentrations: 0.500, 1.50, 8.00 and 80.0 ng/mL

Accuracy and Precision QCs (3 Runs):

| | |
|---|---|
| Intra-Run Accuracy (% bias) | −5.9 → 14.8 |
| Intra-Run Precision (% CV) | 0.6 → 8.1 |
| Inter-Run Accuracy (% bias) | −2.4 → 3.7 |
| Inter-Run Precision (% CV) | 3.3 → 8.4 |

Stability in Treated Human Saliva: 2 Days at −20° C.

Matrix Effects on Quantification: Passed (1 lot of untreated human saliva)

Stability in Untreated Human Saliva: 1 Month at −20° C.; 1 Month at −80° C.

Deoxycholic Acid: Assay Performance

Accepted Calibration Range: 0.500 to 100 ng/mL

Calibration Standard Performance: Inter-Run % Bias −4.7→3.5; Inter-Run % CV 4.9→7.8

Quality Control Concentrations: 0.500, 1.50, 8.00 and 80.0 ng/mL

Accuracy and Precision QCs (3 Runs):

| | |
|---|---|
| Intra-Run Accuracy (% bias) | −1.0 → 20.6 |
| Intra-Run Precision (% CV) | 2.0 → 9.5 |
| Inter-Run Accuracy (% bias) | 3.4 → 8.4 |
| Inter-Run Precision (% CV) | 5.3 → 11.1 |

Stability in Treated Human Saliva: 2 Days at −20° C.

Matrix Effects on Quantification: Passed (1 lot of untreated human saliva)

Stability in Untreated Human Saliva: 1 Month at −20° C.; 1 Month at −80° C.

Glycodeoxycholic Acid: Assay Performance

Accepted Calibration Range: 0.500 to 100 ng/mL

Calibration Standard Performance:

| | |
|---|---|
| Inter-Run % Bias | −2.7 → 4.9 |
| Inter-Run % CV | 2.9 → 7.9 |

Quality Control Concentrations: 0.500, 1.50, 8.00 and 80.0 ng/mL

Accuracy and Precision QCs (3 Runs):

| | |
|---|---|
| Intra-Run Accuracy (% bias) | −2.7 → 19.5 |
| Intra-Run Precision (% CV) | 0.9 → 9.0 |
| Inter-Run Accuracy (% bias) | 3.6 → 10.6 |
| Inter-Run Precision (% CV) | 6.2 → 8.6 |

Stability in Treated Human Saliva: 2 Days at −20° C.

Matrix Effects on Quantification: Passed (1 lot of untreated human saliva)

Stability in Untreated Human Saliva: 1 Month at −20° C.; 1 Month at −80° C.

Lithocholic Acid: Assay Performance

Accepted Calibration Range: 10.0 to 1000 ng/mL

Calibration Standard Performance:

| | |
|---|---|
| Inter-Run % Bias | −4.8 → 3.7 |
| Inter-Run % CV | 7.6 → 12.7 |

Quality Control Concentrations: 15.0, 80.0 and 800 ng/mL

Accuracy and Precision QCs (3 Runs):

| | |
|---|---|
| Intra-Run Accuracy (% bias) | −3.9 → 24.0 |
| Intra-Run Precision (% CV) | 3.3 → 19.1 |
| Inter-Run Accuracy (% bias) | 6.9 → 16.1 |
| Inter-Run Precision (% CV) | 8.5 → 13.6 |

Stability in Treated Human Saliva: 2 Days at −20° C. (15.0 to 800 ng/mL)

Matrix Effects on Quantification: Passed (1 lot of untreated human saliva)

Stability in Untreated Human Saliva: Failed 1 Month at −20° C.

Failed 1 Month at −80° C.

Taurodeoxycholic Acid: Assay Performance

Accepted Calibration Range: 0.500 to 100 ng/mL

Calibration Standard Performance:

| | |
|---|---|
| Inter-Run % Bias | −2.1 → 4.7 |
| Inter-Run % CV | 2.4 → 8.0 |

Quality Control Concentrations: 0.500, 1.50, 8.00 and 80.0 ng/mL

Accuracy and Precision QCs (3 Runs):

| | |
|---|---|
| Intra-Run Accuracy (% bias) | −0.6 → 20.5 |
| Intra-Run Precision (% CV) | 1.0 → 10.7 |
| Inter-Run Accuracy (% bias) | 4.1 → 10.5 |
| Inter-Run Precision (% CV) | 4.9 → 8.3 |

Stability in Treated Human Saliva: 2 Days at −20° C.

Matrix Effects on Quantification: Passed (1 lot of untreated human saliva)

Stability in Untreated Human Saliva: 1 Month at −20° C.; 1 Month at −80° C.

Taurocholic Acid: Assay Performance

Accepted Calibration Range: 0.500 to 100 ng/mL

Calibration Standard Performance:

| | |
|---|---|
| Inter-Run % Bias | −1.5 → 3.2 |
| Inter-Run % CV | 2.8 → 7.2 |

Quality Control Concentrations: 0.500, 1.50, 8.00 and 80.0 ng/mL

Accuracy and Precision QCs (3 Runs):

| | |
|---|---|
| Intra-Run Accuracy (% bias) | −6.6 → 19.3 |
| Intra-Run Precision (% CV) | 0.8 → 7.7 |
| Inter-Run Accuracy (% bias) | −1.9 → 7.3 |
| Inter-Run Precision (% CV) | 4.4 → 9.6 |

Stability in Treated Human Saliva: 2 Days at −20° C.

Matrix Effects on Quantification: Passed (1 lot of untreated human saliva)

Stability in Untreated Human Saliva: 1 Month at −20° C.; 1 Month at −80° C.

Glycochenodeoxycholic Acid: Assay Performance

Accepted Calibration Range: 0.500 to 100 ng/mL

Calibration Standard Performance:

| | |
|---|---|
| Inter-Run % Bias | −2.2 → 3.8 |
| Inter-Run % CV | 2.9 → 7.3 |

Quality Control Concentrations: 0.500, 1.50, 8.00 and 80.0 ng/mL

Accuracy and Precision QCs (3 Runs):

| | |
|---|---|
| Intra-Run Accuracy (% bias) | −2.1 → 28.9 |
| Intra-Run Precision (% CV) | 0.5 → 10.1 |
| Inter-Run Accuracy (% bias) | 2.3 → 13.3 |
| Inter-Run Precision (% CV) | 3.1 → 11.7 |

Stability in Treated Human Saliva: 2 Days at −20° C.

Matrix Effects on Quantification: Passed (1 lot of untreated human saliva)

Stability in Untreated Human Saliva: 1 Month at −20° C.; 1 Month at −80° C.

Taurochenodeoxycholic Acid: Assay Performance

Accepted Calibration Range: 0.500 to 100 ng/mL

Calibration Standard Performance:

| | |
|---|---|
| Inter-Run % Bias | −2.7 → 5.1 |
| Inter-Run % CV | 1.8 → 6.9 |

Quality Control Concentrations: 0.500, 1.50, 8.00 and 80.0 ng/mL

Accuracy and Precision QCs (3 Runs): including failed QC-LLOQ in Run 31

| | |
|---|---|
| Intra-Run Accuracy (% bias) | −3.4 → 43.8 |
| Intra-Run Precision (% CV) | 1.4 → 10.8 |
| Inter-Run Accuracy (% bias) | 8.7 → 20.5 |
| Inter-Run Precision (% CV) | 4.1 → 14.7 |

Stability in Treated Human Saliva: 2 Days at −20° C.

Matrix Effects on Quantification: Passed (1 lot of untreated human saliva)

Stability in Untreated Human Saliva: 1 Month at −20° C.; 1 Month at −80° C.

Abbreviations

BQL: Below quantitation limit; CV: Coefficient of variation; Dil: Dilution; ID: Identification; IS: Internal standard; LC-MS/MS: Liquid chromatography with tandem mass spectrometry; N: Number of samples; MPA: Mobile phase A; MPB: Mobile phase B; NA: Not applicable; QC: Quality control; SD: Standard deviation; Std: Standard.

The objective of this study was to qualify an analytical method for quantification of bile acids (cholic acid, chenodeoxycholic acid, glycocholic acid, deoxycholic acid, glycodeoxycholic acid, lithocholic acid, taurodeoxycholic acid, taurocholic acid, glycochenodeoxycholic acid and aurochenodeoxycholic acid) in human saliva using glycocholic acid-d4 as the internal standard.

Materials and Methods

Reference and Internal Standards

Reference Standard Cholic acid; Manufacturer/Supplier: Sigma-Aldrich; Batch Number MKBR9198V; Storage Conditions Ambient

Reference Standard Chenodeoxycholic acid; Manufacturer/Supplier Santa Cruz Biotechnology, Inc.; Lot Number K1514; Storage Conditions Ambient

Reference Standard: Glycocholic acid hydrate; Manufacturer/Supplier: Sigma-Aldrich; Batch Number SLBH5157V; Storage Conditions Ambient

Reference Standard Deoxycholic acid Manufacturer/Supplier Sigma-Aldrich Batch Number BCBN9953V Storage Conditions Ambient

Reference Standard Glycodeoxycholic acid; Manufacturer/Supplier IsoSciences, LLC; Lot Number EH1-2014-028A1; Storage Conditions −20° C., desiccated

Reference Standard Lithocholic acid; Manufacturer/Supplier IsoSciences, LLC; Lot Number EH1-2014-030A1; Storage Conditions −20° C., desiccated

Reference Standard Sodium taurodeoxycholate hydrate; Manufacturer/Supplier: Sigma-Aldrich Batch Number SLBJ4610V; Storage Conditions Ambient

Reference Standard Taurocholic acid sodium salt hydrate Manufacturer/Supplier: Sigma-Aldrich Batch Number SLBH5200V Storage Conditions Ambient

Reference Standard Sodium Glycochenodeoxycholate; Manufacturer/Supplier: Sigma-Aldrich; Batch Number SLBG7615V; Storage Conditions Ambient

Reference Standard Sodium Taurochenodeoxycholate; Manufacturer/Supplier: Sigma-Aldrich; Batch Number SLBH9352V; Storage Conditions Ambient

Internal Standard Glycocholic Acid-d4; Manufacturer/Supplier: C/D/N Isotopes, Inc.; Lot Number R376P48; Storage Conditions Ambient

Blank Matrix

Saliva was collected from human volunteers and then pooled. Due to endogenous levels of bile acids, pooled saliva was stripped of endogenous bile acids by treatment with 2 mg/mL of cholestyramine resin (Sigma Lot No. 1425455V) for 1 hour at 37° C. followed by centrifugation. The treatment/centrifugation cycle was repeated four times for a total of five cycles. After the final treatment, the treated saliva was pooled for use in preparation of calibration standards and quality control samples.

Preparation of Calibration Standards

Calibration standards were prepared at concentrations of 0.500, 0.750, 1.00, 2.00, 5.00, 10.0, 50.0, 75.0 and 100 ng/mL cholic acid, glycocholic acid, deoxycholic acid, glycodeoxycholic acid, taurodeoxycholic acid, taurocholic acid, glycochenodeoxycholic acid, taurochenodeoxycholic acid and 5.00, 7.50, 10.0, 20.0, 50.0, 100, 500, 750 and 1000 ng/mL chenodeoxycholic acid and lithocholic acid in blank matrix. Standards were prepared on the day of sample extraction and were analyzed in duplicate in each analytical run. Following initial method development and discussions, the decision was made that calibration standards are to be prepared in small volumes for analysis.

Preparation of QC Samples

Quality control samples were prepared containing cholic acid, glycocholic acid, deoxycholic acid, glycodeoxycholic acid, taurodeoxycholic acid, taurocholic acid, glycochenodeoxycholic acid, taurochenodeoxycholic acid at 0.500 ng/mL (QC-LLOQ), 1.50 ng/mL (QC-Low), 8.00 ng/mL (QC-Mid) and 80.0 ng/mL (QC-High) and chenodeoxycholic acid and lithocholic acid at 5.00 ng/mL (QC-LLOQ), 15.0 ng/mL (QC-Low), 80.0 ng/mL (QC-Mid) and 800 ng/mL (QC-High) in blank matrix. Quality control samples for the evaluation of accuracy and precision and for run acceptance were prepared on the day of sample extraction. QC samples prepared in cholestyramine-treated saliva are to be prepared in small volumes for analysis. Stability for these QC samples would be limited to short-term storage (i.e., 2 days) whereas untreated human saliva would be evaluated for long-term storage.

Samples for Frozen Stability

For stability in cholestyramine-treated human saliva, samples were prepared by spiking cholestyramine-treated human saliva with each analyte at the QC-LLOQ, QC-Low, QC-Mid and QC-High levels. The samples were stored in a −20° C. freezer. After 1 day and 2 days of storage, each sample was extracted in replicates of four for analysis.

For long-term stability in untreated human saliva, samples were prepared by spiking untreated human saliva with each analyte at the QC-Low and QC-High levels. The samples were stored in a −20° C. freezer and in a −80° C. freezer. After 1 month of storage, each sample was extracted in replicates of three for analysis.

Samples for Matrix Effects

Matrix effects in untreated human saliva were evaluated by spiking untreated human saliva with each analyte at the QC-Low and QC-High levels and measuring back-calculated concentrations based on calibrants prepared in cholestyramine-treated saliva. The spiked, untreated-saliva samples and a control blank of untreated human saliva (endogenous level) were extracted in replicates of six for analysis.

Sample Extraction

A 100-μL aliquot of sample (calibration standards, quality controls, blanks, and stability samples) was transferred into a 96 well plate, according to a pre-defined layout. Three hundred microliters (300 μL) of ice-cold internal standard spiking solution (2 ng/mL glycocholic acid-d4 in acetonitrile) was added to each sample, except for matrix blanks to which 300 μL of acetonitrile was added. The plates were covered, vortex-mixed and then centrifuged for 5 minutes at 3200 rpm. Supernatants (350 μL each) were transferred into the corresponding wells of a clean 96 well plate and evaporated to dryness under nitrogen in a Turbovap set to 40° C. The dried residue in each well was reconstituted with 75 μL of 50:50 (v:v) methanol:water.

Liquid Chromatography and Mass Spectrometer Conditions

The LC system used was an CTC PAL Autosampler along with Agilent 1260 series pumps. A Hypersil Gold, 1.9 μm column (50×2.1 mm) was used and maintained at 40° C. during analysis. The gradient and mobile phases used are shown below. The flow rate was 0.500 mL/min and the injection volume was 10 μL.

Mobile Phase A: 0.2% (v/v) Formic acid in water

Mobile Phase B: 0.2% (v/v) Formic acid in acetonitrile

Gradient Program:

| Total Time (min) | % MPA | % MPB |
|---|---|---|
| 0.00 | 70 | 30 |
| 0.30 | 50 | 50 |
| 2.27 | 20 | 80 |
| 6.00 | 2 | 98 |
| 7.00 | 2 | 98 |
| 7.10 | 70 | 30 |
| 8.00 | 70 | 30 |

The detector was an Applied Biosystems Sciex API-5500 triple quadrupole mass spectrometer. The instrument was equipped with an electrospray ionization source in positive-ion mode and the analytes were monitored in the multiple-reaction-monitoring scan mode. Q1 and Q3 were operated with unit resolution. The MS/MS transition masses used for the bile acids and internal standard are listed below.

| Compound | Q1 m/z | Q3 m/z |
|---|---|---|
| Cholic acid | 407.1 | 407.1 |
| Chenodeoxycholic acid | 391.2 | 391.2 |
| Glycocholic acid | 464.1 | 74.1 |
| Deoxycholic acid | 391.2 | 391.2 |
| Glycodeoxycholic acid | 448.2 | 74.0 |
| Lithocholic acid | 375.1 | 375.1 |
| Taurodeoxycholic acid | 498.2 | 80.0 |
| Taurocholic acid | 514.2 | 80.0 |
| Glycochenodeoxycholic acid | 448.2 | 74.0 |
| Taurochenodeoxycholic acid | 498.2 | 80.0 |
| Glycocholic acid-d4 | 468.1 | 74.1 |

Data Collection and Analysis

Analyst (Applied Biosystems Sciex) version 1.6 and Aria was used for data acquisition and processing. Descriptive statistics were calculated using Excel (Microsoft).

Run Acceptance Criteria

Data were considered acceptable if the following criteria were met: At least 75% of the total number of calibration standards are within ±30% of their nominal concentrations. At least two-thirds of the total number of quality control samples and at least 50% of the QC replicate per level are within ±30% of their nominal concentrations.

Results

Cholic Acid

In the five accepted qualification runs, no calibration standards were rejected. Back-calculated concentrations for all calibration standards are reported in Table 34. For the LLOQ-Std (0.500 ng/mL), the % bias for the mean back-calculated concentration was 0.8% (4.6% CV).

The assay method meets the requirements for accuracy and precision. At each concentration, at least two out of four QCs and least two-thirds of all QCs were within ±30.0 from nominal concentration. Accuracy (bias of the mean) was within ±30.0% from nominal concentration within and between runs and precision (CV) was ≤30.0% within and between runs. Results are reported in Table 35.

Short-term stability of cholic acid in cholestyramine-treated human saliva was shown for up to 2 days at 20° C. For each stability sample, two of four replicates were within ±30.0% from nominal concentration and accuracy (bias) of the mean value was within ±30.0% with precision (CV)≤30.0%. Results are reported in Table 36.

Results for the evaluation of matrix effects on quantification were acceptable. For the lot of untreated human saliva, six out of six replicates at each concentration were within ±30.0% from nominal concentration, the accuracy (bias) of the mean value was within ±30.0% from nominal concentration, and the precision (CV) was ≤30.0%. Results are reported in Table 37.

Long-term stability of cholic acid in untreated human saliva was shown for up to 1 month at 20° C. and for up to 1 month at 80° C. The mean measured concentration for each stored sample was within ±30% from the mean measured concentration for freshly prepared QC samples (Batch 34). Results are reported in Table 38. Results for batch acceptance quality controls are reported in Table 39.

Chenodeoxycholic Acid

In the five accepted qualification runs, no calibration standards were rejected. Back-calculated concentrations for all calibration standards are reported in Table 40. For the LLOQ-Std (5.00 ng/mL), the % bias for the mean back-calculated concentration was −5.9% (6.6% CV).

The assay method meets the requirements for accuracy and precision. At each concentration, at least two out of four QCs and least two-thirds of all QCs were within ±30.0 from nominal concentration. Accuracy (bias of the mean) was within ±30.0% from nominal concentration within and between runs and precision (CV) was ≤30.0% within and between runs. Results are reported in Table 41.

Short-term stability of chenodeoxycholic acid in cholestyramine-treated human saliva was shown for up to 2 days at 20° C. For each stability sample, two of four replicates were within ±30.0% from nominal concentration and accuracy (bias) of the mean value was within ±30.0% with precision (CV)≤30.0%. Results are reported in Table 42.

Results for the evaluation of matrix effects on quantification were acceptable. For the lot of untreated human saliva, six out of six replicates at each concentration were within ±30.0% from nominal concentration, the accuracy (bias) of the mean value was within ±30.0% from nominal concentration, and the precision (CV) was ≤30.0%. Results are reported in Table 43.

Long-term stability of chenodeoxycholic acid in untreated human saliva was shown for up to 1 month at 20° C. and for up to 1 month at 80° C. The mean measured concentration for each stored sample was within ±30% from the mean measured concentration for freshly prepared QC samples (Batch 34). Results are reported in Table 44. Results for batch acceptance quality controls are reported in Table 45.

Glycocholic Acid

In the five accepted qualification runs, no calibration standards were rejected. Back-calculated concentrations for all calibration standards are reported in Table 46. For the LLOQ-Std (0.500 ng/mL), the % bias for the mean back-calculated concentration was 0.5% (2.6% CV).

The assay method meets the requirements for accuracy and precision. At each concentration, at least two out of four QCs and least two-thirds of all QCs were within ±30.0 from nominal concentration. Accuracy (bias of the mean) was within ±30.0% from nominal concentration within and between runs and precision (CV) was ≤30.0% within and between runs. Results are reported in Table 47.

Short-term stability of glycocholic acid in cholestyramine-treated human saliva was shown for up to 2 days at 20° C. For each stability sample, two of four replicates were within ±30.0% from nominal concentration and accuracy (bias) of the mean value was within ±30.0% with precision (CV) ≤30.0%. Results are reported in Table 48.

Results for the evaluation of matrix effects on quantification were acceptable. For the lot of untreated human saliva, six out of six replicates at each concentration were within ±30.0% from nominal concentration, the accuracy (bias) of the mean value was within ±30.0% from nominal concentration, and the precision (CV) was ≤30.0%. Results are reported in Table 49.

Long-term stability of glycocholic acid in untreated human saliva was shown for up to 1 month at 20° C. and for up to 1 month at 80° C. The mean measured concentration for each stored sample was within ±30% from the mean measured concentration for freshly prepared QC samples (Batch 34). Results are reported in Table 50. Results for batch acceptance quality controls are reported in Table 51.

Deoxycholic Acid

In the five accepted qualification runs, no calibration standards were rejected. Back-calculated concentrations for all calibration standards are reported in Table 52. For the LLOQ-Std (0.500 ng/mL), the % bias for the mean back-calculated concentration was 0.3% (6.0% CV).

The assay method meets the requirements for accuracy and precision. At each concentration, at least two out of four QCs and least two-thirds of all QCs were within ±30.0 from nominal concentration. Accuracy (bias of the mean) was within ±30.0% from nominal concentration within and between runs and precision (CV) was ≤30.0% within and between runs. Results are reported in Table 53.

Short-term stability of deoxycholic acid in cholestyramine-treated human saliva was shown for up to 2 days at 20° C. For each stability sample, two of four replicates were within ±30.0% from nominal concentration and accuracy (bias) of the mean value was within ±30.0% with precision (CV) ≤30.0%. Results are reported in Table 54.

Results for the evaluation of matrix effects on quantification were acceptable. For the lot of untreated human saliva, six out of six replicates at each concentration were within ±30.0% from nominal concentration, the accuracy (bias) of the mean value was within ±30.0% from nominal concentration, and the precision (CV) was ≤30.0%. Results are reported in Table 55.

Long-term stability of deoxycholic acid in untreated human saliva was shown for up to 1 month at 20° C. and for up to 1 month at 80° C. The mean measured concentration for each stored sample was within ±30% from the mean measured concentration for freshly prepared QC samples (Batch 34). Results are reported in Table 56. Results for batch acceptance quality controls are reported in Table 57.

Glycodeoxycholic Acid

In the five accepted qualification runs, no calibration standards were rejected. Back-calculated concentrations for all calibration standards are reported in Table 58. For the LLOQ-Std (0.500 ng/mL), the % bias for the mean back-calculated concentration was −1.3% (2.9% CV).

The assay method meets the requirements for accuracy and precision. At each concentration, at least two out of four QCs and least two-thirds of all QCs were within ±30.0 from nominal concentration. Accuracy (bias of the mean) was within ±30.0% from nominal concentration within and between runs and precision (CV) was ≤30.0% within and between runs Results are reported in Table 59.

Short-term stability of glycodeoxycholic acid in cholestyramine-treated human saliva was shown for up to 2 days at 20° C. For each stability sample, two of four replicates were within ±30.0% from nominal concentration and accuracy (bias) of the mean value was within ±30.0% with precision (CV)≤30.0%. Results are reported in Table 60.

Results for the evaluation of matrix effects on quantification were acceptable. For the lot of untreated human saliva, six out of six replicates at each concentration were within ±30.0% from nominal concentration, the accuracy (bias) of the mean value was within ±30.0% from nominal concentration, and the precision (CV) was ≤30.0%. Results are reported in Table 61.

Long-term stability of glycodeoxycholic acid in untreated human saliva was shown for up to 1 month at 20° C. and for up to 1 month at 80° C. The mean measured concentration for each stored sample was within ±30% from the mean measured concentration for freshly prepared QC samples (Batch 34). Results are reported in Table 62. Results for batch acceptance quality controls are reported in Table 63.

Lithocholic Acid

In the five accepted qualification runs with the truncated curve (10.0 to 1000 ng/mL), no calibration standards were rejected. Back-calculated concentrations for all calibration standards are reported in Table 64. For the LLOQ-Std (10.0 ng/mL), the % bias for the mean back-calculated concentration was −1.1% (11.6% CV).

The assay method meets the requirements for accuracy and precision (excluding the QC-LLOQ due to truncated curve. At each concentration, at least two out of four QCs and least two-thirds of all QCs were within ±30.0 from nominal concentration. Accuracy (bias of the mean) was within ±30.0% from nominal concentration within and between runs and precision (CV) was ≤30.0% within and between runs. Results are reported in Table 65.

Short-term stability of lithocholic acid in cholestyramine-treated human saliva was shown for up to 2 days at 20° C. For each stability sample, two of four replicates were within ±30.0% from nominal concentration and accuracy (bias) of the mean value was within ±30.0% with precision (CV) was ≤30.0%. Results are reported in Table 66.

Results for the evaluation of matrix effects on quantification were acceptable. For the lot of untreated human saliva, six out of six replicates at each concentration were within ±30.0% from nominal concentration, the accuracy (bias) of the mean value was within ±30.0% from nominal concentration, and the precision (CV) was ≤30.0%. Results are reported in Table 67.

Long-term stability of lithocholic acid in untreated human saliva for 1 month at 20° C. or for 1 month at −80° C. was not shown. For both storage temperatures, the mean measured concentration at the QC-Low level was not within ±30% from the mean measured concentration for freshly prepared QC samples (Batch 34). Results are reported in Table 68. Results for batch acceptance quality controls are reported in Table 69.

Taurodeoxycholic Acid

In the five accepted qualification runs, no calibration standards were rejected. Back-calculated concentrations for all calibration standards are reported in Table 70. For the LLOQ-Std (0.500 ng/mL), the % bias for the mean back-calculated concentration was 0.8% (2.4% CV).

The assay method meets the requirements for accuracy and precision. At each concentration, at least two out of four QCs and least two-thirds of all QCs were within ±30.0 from nominal concentration. Accuracy (bias of the mean) was within ±30.0% from nominal concentration within and between runs and precision (CV) was ≤30.0% within and between runs. Results are reported in Table 71.

Short-term stability of taurodeoxycholic acid in cholestyramine-treated human saliva was shown for up to 2 days at 20° C. For each stability sample, two of four replicates were within ±30.0% from nominal concentration and accuracy (bias) of the mean value was within ±30.0% with precision (CV) was ≤30.0%. Results are reported in Table 72.

Results for the evaluation of matrix effects on quantification were acceptable. For the lot of untreated human saliva, six out of six replicates at each concentration were within ±30.0% from nominal concentration, the accuracy (bias) of the mean value was within ±30.0% from nominal concentration, and the precision (CV) was ≤30.0%. Results are reported in Table 73.

Long-term stability of taurodeoxycholic acid in untreated human saliva was shown for up to 1 month at 20° C. and for up to 1 month at 80° C. The mean measured concentration for each stored sample was within ±30% from the mean measured concentration for freshly prepared QC samples (Batch 34). Results are reported in Table 74. Results for batch acceptance quality controls are reported in Table 75.

Taurocholic Acid

In the five accepted qualification runs, no calibration standards were rejected. Back-calculated concentrations for all calibration standards are reported in Table 76. For the LLOQ-Std (0.500 ng/mL), the % bias for the mean back-calculated concentration was 1.0% (3.4% CV).

The assay method meets the requirements for accuracy and precision. At each concentration, at least two out of four QCs and least two-thirds of all QCs were within ±30.0 from nominal concentration. Accuracy (bias of the mean) was within ±30.0% from nominal concentration within and between runs and precision (CV) was ≤30.0% within and between runs. Results are reported in Table 77.

Short-term stability of taurocholic acid in cholestyramine-treated human saliva was shown for up to 2 days at 20° C. For each stability sample, two of four replicates were within ±30.0% from nominal concentration and accuracy (bias) of the mean value was within ±30.0% with precision (CV) ≤30.0%. Results are reported in Table 78.

Results for the evaluation of matrix effects on quantification were acceptable. For the lot of untreated human saliva, six out of six replicates at each concentration were within ±30.0% from nominal concentration, the accuracy (bias) of the mean value was within ±30.0% from nominal concentration, and the precision (CV) was ≤30.0%. Results are reported in Table 79.

Long-term stability of taurocholic acid in untreated human saliva was shown for up to 1 month at 20° C. and for up to 1 month at 80° C. The mean measured concentration for each stored sample was within ±30% from the mean measured concentration for freshly prepared QC samples (Batch 34). Results are reported in Table 80. Results for batch acceptance quality controls are reported in Table 81.

Glycochenodeoxycholic Acid

In the five accepted qualification runs, no calibration standards were rejected. Back-calculated concentrations for all calibration standards are reported in Table 82. For the LLOQ-Std (0.500 ng/mL), the % bias for the mean back-calculated concentration was 0.1% (2.9% CV).

The assay method meets the requirements for accuracy and precision. At each concentration, at least two out of four QCs and least two-thirds of all QCs were within ±30.0 from nominal concentration. Accuracy (bias of the mean) was within ±30.0% from nominal concentration within and between runs and precision (CV) was ≤30.0% within and between runs. Results are reported in Table 83.

Short-term stability of glycochenodeoxycholic acid in cholestyramine-treated human saliva was shown for up to 2 days at 20° C. For each stability sample, two of four replicates were within ±30.0% from nominal concentration and accuracy (bias) of the mean value was within ±30.0% with precision (CV)≤30.0%. Results are reported in Table 84.

Results for the evaluation of matrix effects on quantification were acceptable. For the lot of untreated human saliva, six out of six replicates at each concentration were within ±30.0% from nominal concentration, the accuracy (bias) of the mean value was within ±30.0% from nominal concentration, and the precision (CV) was ≤30.0%. Results are reported in Table 85.

Long-term stability of glycochenodeoxycholic acid in untreated human saliva was shown for up to 1 month at 20° C. and for up to 1 month at 80° C. The mean measured concentration for each stored sample was within ±30% from the mean measured concentration for freshly prepared QC samples (Batch 34). Results are reported in Table 86. Results for batch acceptance quality controls are reported in Table 87.

Taurochenodeoxycholic Acid

In the five accepted qualification runs, no calibration standards were rejected. Back-calculated concentrations for all calibration standards are reported in Table 88. For the LLOQ-Std (0.500 ng/mL), the % bias for the mean back-calculated concentration was 1.8% (3.2% CV).

The assay method meets the requirements for accuracy and precision with the exception of the QC-LLOQ in Run 32. At each concentration, at least two out of four QCs and least two-thirds of all QCs were within ±30.0 from nominal concentration. Accuracy (bias of the mean) was within ±30.0% from nominal concentration within and between runs and precision (CV) was ≤30.0% within and between runs. Results are reported in Table 89.

Short-term stability of taurochenodeoxycholic acid in cholestyramine-treated human saliva was shown for up to 2 days at 20° C. For each stability sample, two of four replicates were within ±30.0% from nominal concentration and accuracy (bias) of the mean value was within ±30.0% with precision (CV)≤30.0%. Results are reported in Table 90.

Results for the evaluation of matrix effects on quantification were acceptable. For the lot of untreated human saliva, six out of six replicates at each concentration were within ±30.0% from nominal concentration, the accuracy (bias) of the mean value was within ±30.0% from nominal concentration, and the precision (CV) was ≤30.0%. Results are reported in Table 91.

Long-term stability of taurochenodeoxycholic acid in untreated human saliva was shown for up to 1 month at 20° C. and for up to 1 month at 80° C. The mean measured concentration for each stored sample was within ±30% from the mean measured concentration for freshly prepared QC samples (Batch 34). Results are reported in Table 92. Results for batch acceptance quality controls are reported in Table 93.

TABLE 34
Cholic Acid: Calibration Standard Back-Calculated Concentrations in
Cholestyramine-Treated Human Saliva

| | Concentration (ng/mL) | | | | | | | | |
|---|---|---|---|---|---|---|---|---|---|
| Analytical | Std-1 | Std-2 | Std-3 | Std-4 | Std-5 | Std-6 | Std-7 | Std-8 | Std-9 |
| Run | 0.500 | 0.750 | 1.00 | 2.00 | 5.00 | 10.0 | 50.0 | 75.0 | 100 |
| B31 | 0.496 | 0.740 | 0.910 | 1.89 | 4.55 | 9.72 | 51.1 | 82.3 | 97.5 |
| | 0.525 | 0.806 | 0.973 | 1.99 | 4.73 | 10.8 | 48.7 | 83.6 | 98.7 |
| B32 | 0.502 | 0.767 | 0.993 | 2.16 | 4.94 | 9.78 | 51.0 | 77.7 | 96.3 |
| | 0.490 | 0.712 | 0.996 | 2.16 | 5.11 | 9.79 | 49.9 | 75.7 | 89.9 |
| B33 | 0.509 | 0.693 | 0.999 | 2.12 | 5.01 | 11.2 | 46.5 | 77.3 | 96.4 |
| | 0.512 | 0.752 | 0.957 | 2.01 | 5.08 | 11.0 | 44.3 | 72.8 | 98.2 |
| B34 | 0.500 | 0.727 | 1.06 | 2.13 | 5.23 | 9.51 | 49.6 | 75.1 | 102 |
| | 0.496 | 0.727 | 0.987 | 2.02 | 5.43 | 9.28 | 49.4 | 72.2 | 97.3 |
| B35 | 0.551 | 0.763 | 0.968 | 2.18 | 4.88 | 10.70 | 52.8 | 76.5 | 96.7 |
| | 0.461 | 0.746 | 0.973 | 1.91 | 4.25 | 10.20 | 46.9 | 81.5 | 99.7 |
| Mean | 0.504 | 0.743 | 0.982 | 2.06 | 4.92 | 10.2 | 49.0 | 77.5 | 97.3 |
| SD | 0.0234 | 0.0317 | 0.0378 | 0.107 | 0.342 | 0.679 | 2.52 | 3.89 | 3.12 |
| % CV | 4.6 | 4.3 | 3.9 | 5.2 | 6.9 | 6.7 | 5.1 | 5.0 | 3.2 |
| % Bias | 0.8 | −0.9 | −1.8 | 2.9 | −1.6 | 2.0 | −2.0 | 3.3 | −2.7 |
| n | 10 | 10 | 10 | 10 | 10 | 10 | 10 | 10 | 10 |

TABLE 35
Cholic Acid: Accuracy and Precision for Quality
Controls (Cholestyramine-Treated Human Saliva)

| | Concentration (ng/mL) | | | |
|---|---|---|---|---|
| | QC-LLOQ | QC-Low | QC-Mid | QC-High |
| Analytical Run | 0.500 | 1.50 | 8.00 | 80.0 |
| 31 | 0.554 | 1.35 | 7.85 | 77.3 |
| | 0.530 | 1.40 | 8.51 | 83.6 |
| | 0.565 | 1.38 | 7.54 | 77.0 |
| | 0.532 | 1.46 | 7.77 | 80.7 |
| Intra-Run Mean | 0.545 | 1.40 | 7.92 | 79.7 |
| Intra-Run SD | 0.0171 | 0.0465 | 0.416 | 3.12 |
| Intra-Run % CV | 3.1 | 3.3 | 5.3 | 3.9 |
| Intra-Run % Bias | 9.1 | −6.8 | −1.0 | −0.4 |
| n | 4 | 4 | 4 | 4 |
| 32 | 0.436 | 1.58 | 7.96 | 82.4 |
| | 0.496 | 1.57 | 7.77 | 76.1 |
| | 0.479 | 1.61 | 7.07 | 79.9 |
| | 0.496 | 1.58 | 8.07 | 79.2 |
| Intra-Run Mean | 0.477 | 1.59 | 7.72 | 79.4 |
| Intra-Run SD | 0.0283 | 0.0173 | 0.449 | 2.59 |
| Intra-Run % CV | 5.9 | 1.1 | 5.8 | 3.3 |
| Intra-Run % Bias | −4.7 | 5.7 | −3.5 | −0.7 |
| n | 4 | 4 | 4 | 4 |
| 33 | 0.470 | 1.54 | 7.85 | 82.1 |
| | 0.479 | 1.50 | 7.90 | 79.0 |
| | 0.521 | 1.59 | 8.25 | 76.5 |
| | 0.527 | 1.60 | 7.52 | 86.0 |
| Intra-Run Mean | 0.499 | 1.56 | 7.88 | 80.9 |
| Intra-Run SD | 0.0289 | 0.0465 | 0.299 | 4.10 |
| Intra-Run % CV | 5.8 | 3.0 | 3.8 | 5.1 |
| Intra-Run % Bias | −0.2 | 3.8 | −1.5 | 1.1 |
| n | 4 | 4 | 4 | 4 |
| Inter-Run Mean | 0.507 | 1.51 | 7.84 | 80.0 |
| Inter-Run SD | 0.0376 | 0.0934 | 0.367 | 3.09 |
| Inter-Run % CV | 7.4 | 6.2 | 4.7 | 3.9 |
| Inter-Run % Bias | 1.4 | 0.9 | −2.0 | 0.0 |
| n | 12 | 12 | 12 | 12 |

TABLE 36
Cholic Acid: Frozen (−20° C.) Stability
in Cholestyramine-Treated Human Saliva

| | | Concentration (ng/mL) | | | |
|---|---|---|---|---|---|
| Storage | Analytical | QC-LLOQ | QC-Low | QC-Mid | QC-High |
| Period | Run | 0.500 | 1.50 | 8.00 | 80.0 |
| 1 Day | 33 | 0.486 | 1.44 | 7.70 | 69.8 |
| | | 0.533 | 1.55 | 7.64 | 75.0 |
| | | 0.466 | 1.65 | 7.09 | 76.1 |
| | | 0.497 | 1.57 | 7.67 | 77.8 |
| | Mean | 0.496 | 1.55 | 7.53 | 74.7 |
| | SD | 0.0281 | 0.0866 | 0.291 | 3.45 |
| | % CV | 5.7 | 5.6 | 3.9 | 4.6 |
| | % Bias | −0.9 | 3.5 | −5.9 | −6.7 |
| | n | 4 | 4 | 4 | 4 |
| 2 Days | 33 | 0.535 | 1.34 | 7.43 | 74.6 |
| | | 0.561 | 1.34 | 7.73 | 77.9 |
| | | 0.555 | 1.37 | 7.80 | 72.3 |
| | | 0.591 | 1.44 | 7.90 | 75.5 |
| | Mean | 0.561 | 1.37 | 7.72 | 75.1 |
| | SD | 0.0232 | 0.0472 | 0.202 | 2.32 |
| | % CV | 4.1 | 3.4 | 2.6 | 3.1 |
| | % Bias | 12.1 | −8.5 | −3.6 | −6.2 |
| | n | 4 | 4 | 4 | 4 |

TABLE 37
Cholic Acid: Matrix Effects on Quantification (Untreated Human Saliva)
Concentration (ng/mL)

| | Cholestyramine-Treated Human | | | | | |
|---|---|---|---|---|---|---|
| | Saliva | | | Untreated Human Saliva | | |
| Analytical | QC-Low | QC-Mid | QC-High | Control | QC-Low | QC-High |
| Run | 1.50 | 8.00 | 80.0 | Blank | 1.50 | 80.0 |
| 34 | 1.57 | 8.57 | 79.2 | BQL | 1.33 | 79.6 |
| | 1.50 | 8.25 | 81.1 | BQL | 1.29 | 79.1 |
| | 1.54 | 7.69 | 78.1 | BQL | 1.23 | 77.0 |
| | NA | NA | NA | BQL | 1.25 | 82.8 |
| | NA | NA | NA | BQL | 1.25 | 80.5 |
| | NA | NA | NA | BQL | 1.32 | 80.7 |
| Mean | 1.54 | 8.17 | 79.5 | BQL | 1.28 | 80.0 |
| SD | 0.0351 | 0.445 | 1.52 | NA | 0.0412 | 1.93 |
| % CV | 2.3 | 5.5 | 1.9 | NA | 3.2 | 2.4 |
| % Bias | 2.4 | 2.1 | −0.7 | NA | −14.8 | −0.1 |
| n | 3 | 3 | 3 | 6 | 6 | 6 |

BQL Below quantitation limit (<0.500 ng/mL)
Note:
Bile acids are detected in some lots of untreated human saliva. The endogenous level for the lot used in this batch was BQL, therefore, the theoretical concentrations for QC-Low and QC-High in untreated human saliva are equal to the spiked concentrations (1.50 and 80.0 ng/mL).

TABLE 38
Cholic Acid: Long-Term (1 Month at −20° C. and at −80°
C.) Stability in Untreated Human Saliva

| | Concentration (ng/mL) | | | |
|---|---|---|---|---|
| | −20° C. Storage | | −80° C. Storage | |
| | QC-Low | QC-High | QC-Low | QC-High |
| Analytical Run | 1.50 | 80.0 | 1.50 | 80.0 |
| 35 | 1.31 | 76.0 | 1.20 | 77.2 |
| | 1.24 | 72.6 | 1.19 | 79.6 |
| | 1.21 | 76.7 | 1.19 | 77.9 |
| Mean | 1.25 | 75.1 | 1.19 | 78.2 |
| SD | 0.0513 | 2.19 | 0.00577 | 1.23 |
| % CV | 4.1 | 2.9 | 0.5 | 1.6 |
| % Bias | −16.4 | −6.1 | −20.4 | −2.2 |
| n | 3 | 3 | 3 | 3 |
| Fresh QCs (Batch 34) | | | | |
| Mean at Time Zero | 1.28 | 80.0 | 1.28 | 80.0 |
| Difference from | −2.1 | −6.1 | −6.8 | −2.2 |
| Fresh (%) | | | | |

TABLE 39
Cholic Acid: Batch Acceptance Quality Controls
(Cholestyramine-Treated Human Saliva)

| | Concentration (ng/mL) | | |
|---|---|---|---|
| | QC-Low | QC-Mid | QC-High |
| Analytical Run | 1.50 | 8.00 | 80.0 |
| 35 | 1.88 | 7.48 | 68.3 |
| | 1.55 | 7.60 | 76.7 |
| | 1.56 | 8.78 | 76.0 |
| Mean | 1.66 | 7.95 | 73.7 |
| SD | 0.1877 | 0.718 | 4.66 |
| % CV | 11.3 | 9.0 | 6.3 |
| % Bias | 10.9 | −0.6 | −7.9 |
| n | 3 | 3 | 3 |

TABLE 40
Chenodeoxycholic Acid: Calibration Standard Back-Calculated Concentrations
in Cholestyramine-Treated Human Saliva

| | Concentration (ng/mL) | | | | | | | | |
|---|---|---|---|---|---|---|---|---|---|
| Analytical | Std-1 | Std-2 | Std-3 | Std-4 | Std-5 | Std-6 | Std-7 | Std-8 | Std-9 |
| Run | 5.00 | 7.50 | 10.0 | 20.0 | 50.0 | 100 | 500 | 750 | 1000 |
| B31 | 4.44 | 7.22 | 9.71 | 21.9 | 46.1 | 95.5 | 534 | 806 | 861 |
| | 5.21 | 8.11 | 10.1 | 22.5 | 51.3 | 106 | 489 | 759 | 864 |
| B32 | 4.40 | 7.81 | 10.2 | 22.7 | 50.7 | 101 | 475 | 714 | 896 |
| | 5.05 | 7.32 | 9.77 | 24.0 | 58.2 | 104 | 483 | 726 | 808 |
| B33 | 4.41 | 7.63 | 9.43 | 23.8 | 52.1 | 115 | 456 | 635 | 835 |
| | 4.95 | 7.58 | 11.0 | 21.6 | 51.6 | 125 | 401 | 657 | 1050 |
| B34 | 4.86 | 7.20 | 10.4 | 22.2 | 57.7 | 105 | 467 | 684 | 888 |
| | 4.76 | 7.08 | 10.5 | 22.0 | 63.9 | 103 | 461 | 652 | 836 |
| B35 | 4.64 | 7.93 | 10.7 | 24.3 | 55.5 | 117 | 488 | 640 | 791 |
| | 4.31 | 7.62 | 10.7 | 20.9 | 50.6 | 116 | 454 | 694 | 824 |
| Mean | 4.70 | 7.55 | 10.3 | 22.6 | 53.8 | 109 | 471 | 697 | 865 |
| SD | 0.311 | 0.340 | 0.502 | 1.12 | 5.08 | 9.05 | 33.8 | 55.5 | 72.9 |
| % CV | 6.6 | 4.5 | 4.9 | 4.9 | 9.5 | 8.3 | 7.2 | 8.0 | 8.4 |
| % Bias | −5.9 | 0.7 | 2.5 | 13.0 | 7.5 | 8.8 | −5.8 | −7.1 | −13.5 |
| n | 10 | 10 | 10 | 10 | 10 | 10 | 10 | 10 | 10 |

TABLE 41
Chenodeoxycholic Acid: Accuracy and Precision for Quality
Controls (Cholestyramine-Treated Human Saliva)

| | Concentration (ng/mL) | | | |
|---|---|---|---|---|
| | QC-LLOQ | QC-Low | QC-Mid | QC-High |
| Analytical Run | 5.00 | 15.0 | 80.0 | 800 |
| 31 | 6.31 | 16.2 | 87.5 | 762 |
| | 5.84 | 17.0 | 94.8 | 787 |
| | 6.81^{a} | 15.1 | 88.5 | 728 |
| | 6.40 | 13.8 | 97.4 | 805 |
| Intra-Run Mean | 6.18 | 15.5 | 92.1 | 771 |
| Intra-Run SD | 0.301 | 1.39 | 4.81 | 33.4 |
| Intra-Run % CV | 4.9 | 8.9 | 5.2 | 4.3 |
| Intra-Run % Bias | 23.7 | 3.5 | 15.1 | −3.7 |
| n | 3 | 4 | 4 | 4 |
| 32 | 4.56 | 18.7 | 85.5 | 843 |
| | 4.75 | 20.0^{a} | 91.7 | 721 |
| | 4.14 | 17.4 | 87.6 | 760 |
| | 5.28 | 18.5 | 101 | 746 |
| Intra-Run Mean | 4.68 | 18.2 | 91.5 | 768 |
| Intra-Run SD | 0.473 | 0.700 | 6.87 | 52.9 |
| Intra-Run % CV | 10.1 | 3.8 | 7.5 | 6.9 |
| Intra-Run % Bias | −6.4 | 21.3 | 14.3 | −4.1 |
| n | 4 | 3 | 4 | 4 |
| 33 | 4.76 | 13.5 | 75.5 | 706 |
| | 4.82 | 14.4 | 85.7 | 671 |
| | 4.06 | 16.5 | 78.5 | 633 |
| | 4.86 | 14.8 | 75.5 | 861 |
| Intra-Run Mean | 4.63 | 14.8 | 78.8 | 718 |
| Intra-Run SD | 0.379 | 1.26 | 4.81 | 100 |
| Intra-Run % CV | 8.2 | 8.5 | 6.1 | 13.9 |
| Intra-Run % Bias | −7.5 | −1.3 | −1.5 | −10.3 |
| n | 4 | 4 | 4 | 4 |
| Inter-Run Mean | 5.07 | 16.0 | 87.4 | 752 |
| Inter-Run SD | 0.800 | 1.81 | 8.14 | 66.6 |
| Inter-Run % CV | 15.8 | 11.3 | 9.3 | 8.9 |
| Inter-Run % Bias | 1.4 | 6.6 | 9.3 | −6.0 |
| n | 11 | 11 | 12 | 12 |

^{a}Failed to meet acceptance criteria (within ±30% from nominal concentration); excluded from statistics.

TABLE 42
Chenodeoxycholic Acid: Frozen (−20° C.) Stability
in Cholestyramine-Treated Human Saliva

| | | Concentration (ng/mL) | | | |
|---|---|---|---|---|---|
| Storage | Analytical | QC-LLOQ | QC-Low | QC-Mid | QC-High |
| Period | Run | 5.00 | 15.0 | 80.0 | 800 |
| 1 Day | 33 | 4.89 | 14.9 | 78.0 | 671 |
| | | 5.28 | 16.2 | 79.2 | 717 |
| | | 4.72 | 16.9 | 85.9 | 644 |
| | | 5.20 | 17.4 | 84.8 | 699 |
| | Mean | 5.02 | 16.4 | 82.0 | 683 |
| | SD | 0.263 | 1.08 | 3.95 | 32.0 |
| | % CV | 5.2 | 6.6 | 4.8 | 4.7 |
| | % Bias | 0.4 | 9.0 | 2.5 | −14.7 |
| | n | 4 | 4 | 4 | 4 |
| 2 Days | 33 | 5.73 | 14.3 | 80.0 | 693 |
| | | 6.15 | 14.3 | 87.1 | 777 |
| | | 5.60 | 14.1 | 88.9 | 725 |
| | | 6.18 | 14.6 | 78.8 | 706 |
| | Mean | 5.92 | 14.3 | 83.7 | 725 |
| | SD | 0.294 | 0.21 | 5.04 | 36.9 |
| | % CV | 5.0 | 1.4 | 6.0 | 5.1 |
| | % Bias | 18.3 | −4.5 | 4.6 | −9.3 |
| | n | 4 | 4 | 4 | 4 |

TABLE 43
Chenodeoxycholic Acid: Matrix Effects on Quantification (Untreated Human Saliva
Concentration (ng/mL)

| | Cholestyramine-Treated | | | Saliva Untreated Human | | |
|---|---|---|---|---|---|---|
| | Human | | | Saliva | | |
| Analytical | QC-Low | QC-Mid | QC-High | Control | QC-Low | QC-High |
| Run | 15.0 | 80.0 | 800 | Blank | 15.0 | 800 |
| 34 | 18.1 | 94.2 | 717 | BQL | 15.5 | 724 |
| | 16.6 | 91.6 | 717 | BQL | 14.4 | 708 |
| | 17.3 | 88.9 | 719 | BQL | 13.8 | 826 |
| | NA | NA | NA | BQL | 14.0 | 703 |
| | NA | NA | NA | BQL | 13.3 | 877 |
| | NA | NA | NA | BQL | 14.2 | 870 |
| Mean | 17.3 | 91.6 | 718 | BQL | 14.2 | 785 |
| SD | 0.751 | 2.65 | 1.15 | NA | 0.740 | 82.2 |
| % CV | 4.3 | 2.9 | 0.2 | NA | 5.2 | 10.5 |
| % Bias | 15.6 | 14.5 | −10.3 | NA | −5.3 | −1.9 |
| n | 3 | 3 | 3 | 6 | 6 | 6 |

BQL Below quantitation limit (<5.00 ng/mL)
Note:
Bile acids are detected in some lots of untreated human saliva. The endogenous level for the lot used in this batch was BQL, therefore, the theoretical concentrations for QC-Low and QC-High in untreated human saliva are equal to the spiked concentrations (15.0 and 800 ng/mL).

TABLE 44
Chenodeoxycholic Acid: Long-Term (1 Month at −20° C. and
at −80° C.) Stability in Untreated Human Saliva

| | Concentration (ng/mL) | | | |
|---|---|---|---|---|
| | −20° C. Storage | | −80° C. Storage | |
| | QC-Low | QC-High | QC-Low | QC-High |
| Analytical Run | 15.0 | 800 | 15.0 | 800 |
| 35 | 16.0 | 659 | 15.1 | 630 |
| | 16.9 | 693 | 14.1 | 701 |
| | 15.9 | 686 | 15.5 | 692 |
| Mean | 16.3 | 679 | 14.9 | 674 |
| SD | 0.551 | 18.0 | 0.721 | 38.7 |
| % CV | 3.4 | 2.6 | 4.8 | 5.7 |
| % Bias | 8.4 | −15.1 | −0.7 | −15.7 |
| n | 3 | 3 | 3 | 3 |
| Fresh QCs (Batch 34) | | | | |
| Mean at Time Zero | 14.2 | 785 | 14.2 | 785 |
| Difference from | 14.6 | −13.5 | 4.9 | −14.1 |
| Fresh (%) | | | | |

TABLE 45
Chenodeoxycholic Acid: Batch Acceptance Quality
Controls (Cholestyramine-Treated Human Saliva)

| | Concentration (ng/mL) | | |
|---|---|---|---|
| | QC-Low | QC-Mid | QC-High |
| Analytical Run | 15.0 | 80.0 | 800 |
| 35 | 22.3^{a} | 95.8 | 655 |
| | 17.7 | 92.9 | 753 |
| | 18.2 | 108 | 712 |
| Mean | 18.0 | 98.9 | 707 |
| SD | 0.354 | 8.01 | 49.2 |
| % CV | 2.0 | 8.1 | 7.0 |
| % Bias | 19.7 | 23.6 | −11.7 |
| n | 2 | 3 | 3 |

^{a}Failed to meet acceptance criteria (within ±30% from nominal concentration); excluded from statistics.

TABLE 46
Glycocholic Acid: Calibration Standard Back-Calculated Concentrations in
Cholestyramine-Treated Human Saliva

| | Concentration (ng/mL) | | | | | | | | |
|---|---|---|---|---|---|---|---|---|---|
| Analytical | Std-1 | Std-2 | Std-3 | Std-4 | Std-5 | Std-6 | Std-7 | Std-8 | Std-9 |
| Run | 0.500 | 0.750 | 1.00 | 2.00 | 5.00 | 10.0 | 50.0 | 75.0 | 100 |
| B31 | 0.517 | 0.769 | 0.966 | 1.93 | 4.54 | 9.92 | 51.5 | 83.4 | 95.8 |
| | 0.501 | 0.732 | 0.995 | 2.05 | 4.61 | 10.2 | 47.9 | 83.1 | 100 |
| B32 | 0.478 | 0.724 | 1.04 | 2.25 | 5.12 | 9.82 | 50.2 | 77.0 | 95.9 |
| | 0.504 | 0.737 | 0.999 | 2.17 | 4.98 | 9.65 | 48.4 | 74.1 | 92.4 |
| B33 | 0.491 | 0.727 | 1.00 | 2.09 | 5.01 | 11.0 | 47.7 | 73.7 | 98.3 |
| | 0.524 | 0.736 | 0.955 | 2.03 | 5.01 | 11.2 | 45.9 | 70.7 | 99.7 |
| B34 | 0.500 | 0.727 | 1.06 | 2.13 | 5.23 | 9.51 | 49.6 | 75.1 | 102 |
| | 0.496 | 0.727 | 0.987 | 2.02 | 5.43 | 9.28 | 49.4 | 72.2 | 97.3 |
| B35 | 0.511 | 0.799 | 0.924 | 2.05 | 4.68 | 10.3 | 52.5 | 76.7 | 98.5 |
| | 0.502 | 0.729 | 0.980 | 2.03 | 4.57 | 10.2 | 47.6 | 81.1 | 101 |
| Mean | 0.502 | 0.741 | 0.991 | 2.08 | 4.92 | 10.1 | 49.1 | 76.7 | 98.1 |
| SD | 0.0130 | 0.0242 | 0.0393 | 0.0896 | 0.305 | 0.616 | 1.98 | 4.47 | 2.85 |
| % CV | 2.6 | 3.3 | 4.0 | 4.3 | 6.2 | 6.1 | 4.0 | 5.8 | 2.9 |
| % Bias | 0.5 | −1.2 | −0.9 | 3.8 | −1.6 | 1.1 | −1.9 | 2.3 | −1.9 |
| n | 10 | 10 | 10 | 10 | 10 | 10 | 10 | 10 | 10 |

TABLE 47
Glycocholic Acid: Accuracy and Precision for Quality
Controls (Cholestyramine-Treated Human Saliva)

| | Concentration (ng/mL) | | | |
|---|---|---|---|---|
| | QC-LLOQ | QC-Low | QC-Mid | QC-High |
| Analytical Run | 0.500 | 1.50 | 8.00 | 80.0 |
| 31 | 0.551 | 1.44 | 8.08 | 80.9 |
| | 0.587 | 1.46 | 8.17 | 82.6 |
| | 0.597 | 1.40 | 7.97 | 76.8 |
| | 0.560 | 1.41 | 8.09 | 81.5 |
| Intra-Run Mean | 0.574 | 1.43 | 8.08 | 80.5 |
| Intra-Run SD | 0.0218 | 0.0275 | 0.0822 | 2.53 |
| Intra-Run % CV | 3.8 | 1.9 | 1.0 | 3.1 |
| Intra-Run % Bias | 14.8 | −4.8 | 1.0 | 0.6 |
| n | 4 | 4 | 4 | 4 |
| 32 | 0.486 | 1.66 | 7.87 | 85.3 |
| | 0.482 | 1.57 | 7.75 | 76.7 |
| | 0.484 | 1.53 | 7.72 | 81.4 |
| | 0.489 | 1.62 | 7.95 | 77.9 |
| Intra-Run Mean | 0.485 | 1.60 | 7.82 | 80.3 |
| Intra-Run SD | 0.00299 | 0.0569 | 0.107 | 3.87 |
| Intra-Run % CV | 0.6 | 3.6 | 1.4 | 4.8 |
| Intra-Run % Bias | −3.0 | 6.3 | −2.2 | 0.4 |
| n | 4 | 4 | 4 | 4 |
| 33 | 0.488 | 1.52 | 7.42 | 81.1 |
| | 0.509 | 1.56 | 7.66 | 76.9 |
| | 0.480 | 1.59 | 7.69 | 72.1 |
| | 0.509 | 1.55 | 7.34 | 87.3 |
| Intra-Run Mean | 0.497 | 1.56 | 7.53 | 79.4 |
| Intra-Run SD | 0.0148 | 0.0289 | 0.174 | 6.45 |
| Intra-Run % CV | 3.0 | 1.9 | 2.3 | 8.1 |
| Intra-Run % Bias | −0.7 | 3.7 | −5.9 | −0.8 |
| n | 4 | 4 | 4 | 4 |
| Inter-Run Mean | 0.519 | 1.53 | 7.81 | 80.0 |
| Inter-Run SD | 0.0434 | 0.0830 | 0.261 | 4.18 |
| Inter-Run % CV | 8.4 | 5.4 | 3.3 | 5.2 |
| Inter-Run % Bias | 3.7 | 1.7 | −2.4 | 0.1 |
| n | 12 | 12 | 12 | 12 |

TABLE 48
Glycocholic Acid: Frozen (−20° C.) Stability
in Cholestyramine-Treated Human Saliva

| | | Concentration (ng/mL) | | | |
|---|---|---|---|---|---|
| Storage | Analytical | QC-LLOQ | QC-Low | QC-Mid | QC-High |
| Period | Run | 0.500 | 1.50 | 8.00 | 80.0 |
| 1 Day | 33 | 0.464 | 1.43 | 7.28 | 73.6 |
| | | 0.541 | 1.51 | 7.01 | 74.2 |
| | | 0.485 | 1.56 | 7.20 | 73.2 |
| | | 0.474 | 1.45 | 7.21 | 74.9 |
| | Mean | 0.491 | 1.49 | 7.18 | 74.0 |
| | SD | 0.0344 | 0.0591 | 0.116 | 0.741 |
| | % CV | 7.0 | 4.0 | 1.6 | 1.0 |
| | % Bias | −1.8 | −0.8 | −10.3 | −7.5 |
| | n | 4 | 4 | 4 | 4 |
| 2 Days | 33 | 0.464 | 1.43 | 7.28 | 73.6 |
| | | 0.541 | 1.51 | 7.01 | 74.2 |
| | | 0.485 | 1.56 | 7.20 | 73.2 |
| | | 0.474 | 1.45 | 7.21 | 74.9 |
| | Mean | 0.542 | 1.29 | 7.34 | 74.7 |
| | SD | 0.0197 | 0.0250 | 0.234 | 0.650 |
| | % CV | 3.6 | 1.9 | 3.2 | 0.9 |
| | % Bias | 8.5 | −13.8 | −8.3 | −6.7 |
| | n | 4 | 4 | 4 | 4 |

TABLE 49
Glycocholic Acid: Matrix Effects on Quantification
(Untreated Human Saliva)

| | Concentration (ng/mL) | | | | | |
|---|---|---|---|---|---|---|
| | Cholestyramine- | | | Untreated | | |
| | Treated Human Saliva | | | Human Saliva | | |
| | QC- | QC- | QC- | | QC- | QC- |
| Analytical | Low | Mid | High | Control | Low | High |
| Run | 1.50 | 8.00 | 80.0 | Blank | 1.50 | 80.0 |
| 34 | 1.47 | 8.56 | 81.1 | BQL | 1.35 | 80.6 |
| | 1.59 | 8.30 | 81.4 | BQL | 1.27 | 77.6 |
| | 1.52 | 8.04 | 80.8 | BQL | 1.35 | 79.9 |
| | NA | NA | NA | BQL | 1.30 | 82.1 |
| | NA | NA | NA | BQL | 1.26 | 80.5 |
| | NA | NA | NA | BQL | 1.33 | 81.4 |
| Mean | 1.53 | 8.30 | 81.1 | BQL | 1.31 | 80.4 |
| SD | 0.0603 | 0.260 | 0.300 | NA | 0.0395 | 1.55 |
| % CV | 3.9 | 3.1 | 0.4 | NA | 3.0 | 1.9 |
| % Bias | 1.8 | 3.7 | 1.4 | NA | −12.7 | 0.4 |
| n | 3 | 3 | 3 | 6 | 6 | 6 |

BQL Below quantitation limit (<0.500 ng/mL)
Note:
Bile acids are detected in some lots of untreated human saliva. The endogenous level for the lot used in this batch was BQL, therefore, the theoretical concentrations for QC-Low and QC-High in untreated human saliva are equal to the spiked concentrations (1.50 and 80.0 ng/mL).

TABLE 50
Glycocholic Acid: Long-Term (1 Month at −20° C. and at −80°
C.) Stability in Untreated Human Saliva

| | Concentration (ng/mL) | | | |
|---|---|---|---|---|
| | −20° C. Storage | | −80° C. Storage | |
| Analytical | QC-Low | QC-High | QC-Low | QC-High |
| Run | 1.50 | 80.0 | 1.50 | 80.0 |
| 35 | 1.15 | 77.5 | 1.12 | 74.1 |
| | 1.33 | 72.8 | 1.18 | 78.4 |
| | 1.24 | 75.6 | 1.18 | 75.9 |
| Mean | 1.24 | 75.3 | 1.16 | 76.1 |
| SD | 0.0900 | 2.36 | 0.0346 | 2.16 |
| % CV | 7.3 | 3.1 | 3.0 | 2.8 |
| % Bias | −17.3 | −5.9 | −22.7 | −4.8 |
| n | 3 | 3 | 3 | 3 |
| Fresh QCs (Batch 34) | | | | |
| Mean at Time Zero | 1.31 | 80.4 | 1.31 | 80.4 |
| Difference from | −5.3 | −6.3 | −11.5 | −5.3 |
| Fresh (%) | | | | |

TABLE 51
Glycocholic Acid: Batch Acceptance Quality Controls
(Cholestyramine-Treated Human Saliva)

| | Concentration (ng/mL) | | |
|---|---|---|---|
| Analytical | QC-Low | QC-Mid | QC-High |
| Run | 1.50 | 8.00 | 80.0 |
| 35 | 1.93 | 7.99 | 75.2 |
| | 1.77 | 7.61 | 83.1 |
| | 1.61 | 8.94 | 79.2 |
| Mean | 1.77 | 8.18 | 79.2 |
| SD | 0.160 | 0.685 | 3.95 |
| % CV | 9.0 | 8.4 | 5.0 |
| % Bias | 18.0 | 2.3 | −1.0 |
| n | 3 | 3 | 3 |

TABLE 52
Deoxycholic Acid: Calibration Standard Back-Calculated Concentrations in
Cholestyramine-Treated Human Saliva

| | Concentration (ng/mL) | | | | | | | | |
|---|---|---|---|---|---|---|---|---|---|
| | Std-1 | Std-2 | Std-3 | Std-4 | Std-5 | Std-6 | Std-7 | Std-8 | Std-9 |
| Analytical Run | 0.500 | 0.750 | 1.00 | 2.00 | 5.00 | 10.0 | 50.0 | 75.0 | 100 |
| B31 | 0.474 | 0.744 | 1.05 | 1.76 | 4.36 | 9.61 | 51.2 | 80.7 | 91.5 |
| | 0.511 | 0.820 | 0.967 | 2.07 | 4.75 | 10.8 | 49.7 | 85.2 | 100 |
| B32 | 0.504 | 0.760 | 0.974 | 2.04 | 4.69 | 10.4 | 51.9 | 77.6 | 102 |
| | 0.491 | 0.749 | 1.04 | 2.01 | 4.65 | 9.46 | 54.1 | 78.0 | 89.1 |
| B33 | 0.481 | 0.686 | 1.060 | 2.28 | 4.46 | 10.8 | 48.4 | 76.8 | 100 |
| | 0.532 | 0.783 | 0.924 | 1.87 | 4.74 | 11.2 | 42.6 | 72.0 | 111 |
| B34 | 0.457 | 0.722 | 0.970 | 1.98 | 5.13 | 9.25 | 51.9 | 77.6 | 100 |
| | 0.550 | 0.775 | 1.01 | 1.96 | 5.60 | 9.58 | 47.9 | 73.3 | 101 |
| B35 | 0.535 | 0.822 | 1.020 | 2.21 | 4.86 | 10.5 | 51.0 | 74.7 | 98.6 |
| | 0.479 | 0.670 | 0.927 | 1.91 | 4.40 | 10.3 | 48.3 | 80.7 | 99.2 |
| Mean | 0.501 | 0.753 | 0.994 | 2.01 | 4.76 | 10.2 | 49.7 | 77.7 | 99.2 |
| SD | 0.0303 | 0.0507 | 0.0490 | 0.154 | 0.373 | 0.670 | 3.17 | 3.89 | 5.91 |
| % CV | 6.0 | 6.7 | 4.9 | 7.7 | 7.8 | 6.6 | 6.4 | 5.0 | 6.0 |
| % Bias | 0.3 | 0.4 | −0.6 | 0.5 | −4.7 | 1.9 | −0.6 | 3.5 | −0.8 |
| n | 10 | 10 | 10 | 10 | 10 | 10 | 10 | 10 | 10 |

TABLE 53
Deoxycholic Acid: Accuracy and Precision for Quality
Controls (Cholestyramine-Treated Human Saliva)

| | Concentration (ng/mL) | | | |
|---|---|---|---|---|
| Analytical | QC-LLOQ | QC-Low | QC-Mid | QC-High |
| Run | 0.500 | 1.50 | 8.00 | 80.0 |
| 31 | 0.569 | 1.59 | 8.38 | 84.7 |
| | 0.641 | 1.55 | 8.72 | 92.1 |
| | 0.580 | 1.50 | 7.90 | 74.0 |
| | 0.622 | 1.47 | 8.14 | 90.1 |
| Intra-Run Mean | 0.603 | 1.53 | 8.29 | 85.2 |
| Intra-Run SD | 0.0341 | 0.0532 | 0.350 | 8.11 |
| Intra-Run % CV | 5.7 | 3.5 | 4.2 | 9.5 |
| Intra-Run % Bias | 20.6 | 1.8 | 3.6 | 6.5 |
| n | 4 | 4 | 4 | 4 |
| 32 | 0.438 | 1.70 | 7.70 | 84.8 |
| | 0.479 | 1.62 | 8.46 | 84.6 |
| | 0.523 | 1.60 | 7.43 | 81.0 |
| | 0.541 | 1.71 | 8.17 | 79.6 |
| Intra-Run Mean | 0.495 | 1.66 | 7.94 | 82.5 |
| Intra-Run SD | 0.0462 | 0.0556 | 0.462 | 2.61 |
| Intra-Run % CV | 9.3 | 3.4 | 5.8 | 3.2 |
| Intra-Run % Bias | −1.0 | 10.5 | −0.8 | 3.1 |
| n | 4 | 4 | 4 | 4 |
| 33 | 0.549 | 1.67 | 9.12 | 94.3 |
| | 0.500 | 1.74 | 8.36 | 90.3 |
| | 0.497 | 1.67 | 8.16 | 81.5 |
| | 0.499 | 1.69 | 8.72 | 99.2 |
| Intra-Run Mean | 0.511 | 1.69 | 8.59 | 91.3 |
| Intra-Run SD | 0.0252 | 0.0330 | 0.423 | 7.49 |
| Intra-Run % CV | 4.9 | 2.0 | 4.9 | 8.2 |
| Intra-Run % Bias | 2.3 | 12.8 | 7.4 | 14.2 |
| n | 4 | 4 | 4 | 4 |
| Inter-Run Mean | 0.537 | 1.63 | 8.27 | 86.4 |
| Inter-Run SD | 0.0594 | 0.0861 | 0.466 | 7.07 |
| Inter-Run % CV | 11.1 | 5.3 | 5.6 | 8.2 |
| Inter-Run % Bias | 7.3 | 8.4 | 3.4 | 7.9 |
| n | 12 | 12 | 12 | 12 |

TABLE 54
Deoxycholic Acid: Frozen (−20° C.) Stability
in Cholestyramine-Treated Human Saliva

| | | Concentration (ng/mL) | | | |
|---|---|---|---|---|---|
| Storage | Analytical | QC-LLOQ | QC-Low | QC-Mid | QC-High |
| Period | Run | 0.500 | 1.50 | 8.00 | 80.0 |
| 1 Day | 33 | 0.455 | 1.55 | 6.75 | 62.5 |
| | | 0.407 | 1.47 | 7.31 | 70.2 |
| | | 0.501 | 1.42 | 6.57 | 67.7 |
| | | 0.532 | 1.44 | 7.09 | 70.7 |
| | Mean | 0.474 | 1.47 | 6.93 | 67.8 |
| | SD | 0.0546 | 0.0572 | 0.333 | 3.75 |
| | % CV | 11.5 | 3.9 | 4.8 | 5.5 |
| | % Bias | −5.3 | −2.0 | −13.4 | −15.3 |
| | n | 4 | 4 | 4 | 4 |
| 2 Days | 33 | 0.551 | 1.44 | 8.24 | 77.4 |
| | | 0.651^{a} | 1.39 | 7.98 | 79.6 |
| | | 0.548 | 1.41 | 8.25 | 80.4 |
| | | 0.616 | 1.39 | 8.28 | 82.0 |
| | Mean | 0.572 | 1.41 | 8.19 | 79.9 |
| | SD | 0.0384 | 0.0236 | 0.139 | 1.91 |
| | % CV | 6.7 | 1.7 | 1.7 | 2.4 |
| | % Bias | 14.3 | −6.2 | 2.3 | −0.2 |
| | n | 3 | 4 | 4 | 4 |

^{a}Failed to meet acceptance criteria (within ±30% from nominal concentration); excluded from statistics.

TABLE 55
Deoxycholic Acid: Matrix Effects on Quantification
(Untreated Human Saliva)

| | Concentration (ng/mL) | | | | | |
|---|---|---|---|---|---|---|
| | Cholestyramine-Treated | | | Untreated | | |
| | Human Saliva | | | Human Saliva | | |
| | QC- | QC- | QC- | | QC- | QC- |
| Analytical | Low | Mid | High | Control | Low | High |
| Run | 1.50 | 8.00 | 80.0 | Blank | 1.50 | 80.0 |
| 34 | 1.54 | 8.78 | 80.9 | BQL | 1.41 | 86.0 |
| | 1.51 | 8.39 | 81.7 | BQL | 1.42 | 79.5 |
| | 1.59 | 7.71 | 82.5 | BQL | 1.34 | 78.2 |
| | NA | NA | NA | BQL | 1.34 | 80.4 |
| | NA | NA | NA | BQL | 1.29 | 82.3 |
| | NA | NA | NA | BQL | 1.42 | 81.6 |
| Mean | 1.55 | 8.29 | 81.7 | BQL | 1.37 | 81.3 |
| SD | 0.0404 | 0.542 | 0.800 | NA | 0.0544 | 2.71 |
| % CV | 2.6 | 6.5 | 1.0 | NA | 4.0 | 3.3 |
| % Bias | 3.1 | 3.7 | 2.1 | NA | −8.7 | 1.7 |
| n | 3 | 3 | 3 | 6 | 6 | 6 |

BQL Below quantitation limit (<0.500 ng/mL)
Note:
Bile acids are detected in some lots of untreated human saliva. The endogenous level for the lot used in this batch was BQL, therefore, the theoretical concentrations for QC-Low and QC-High in untreated human saliva are equal to the spiked concentrations (1.50 and 80.0 ng/mL).

TABLE 56
Deoxycholic Acid: Long-Tenn (1 Month at −20° C. and at −80°
C.) Stability in Untreated Human Saliva

| | Concentration (ng/mL) | | | |
|---|---|---|---|---|
| | −20° C. Storage | | −80° C. Storage | |
| Analytical | QC-Low | QC-High | QC-Low | QC-High |
| Run | 1.50 | 80.0 | 1.50 | 80.0 |
| 35 | 1.55 | 78.4 | 1.47 | 75.5 |
| | 1.65 | 74.8 | 1.32 | 78.1 |
| | 1.60 | 77.6 | 1.52 | 83.6 |
| Mean | 1.60 | 76.9 | 1.44 | 79.1 |
| SD | 0.0500 | 1.89 | 0.104 | 4.14 |
| % CV | 3.1 | 2.5 | 7.2 | 5.2 |
| % Bias | 6.7 | −3.8 | −4.2 | −1.2 |
| n | 3 | 3 | 3 | 3 |
| Fresh QCs (Batch 34) | | | | |
| Mean at Time Zero | 1.37 | 81.3 | 1.37 | 81.3 |
| Difference from | 16.8 | −5.4 | 4.9 | −2.7 |
| Fresh (%) | | | | |

TABLE 57
Deoxycholic Acid: Batch Acceptance Quality Controls
(Cholestyramine-Treated Human Saliva)

| | Concentration (ng/mL) | | |
|---|---|---|---|
| Analytical | QC-Low | QC-Mid | QC-High |
| Run | 1.50 | 8.00 | 80.0 |
| 35 | 2.13^{a} | 8.07 | 72.6 |
| | 1.60 | 8.59 | 84.4 |
| | 1.56 | 9.27 | 76.8 |
| Mean | 1.58 | 8.64 | 77.9 |
| SD | 0.0283 | 0.602 | 5.98 |
| % CV | 1.8 | 7.0 | 7.7 |
| % Bias | 5.3 | 8.0 | −2.6 |
| n | 2 | 3 | 3 |

^{a}Failed to meet acceptance criteria (within ±30% from nominal concentration); excluded from statistics.

TABLE 58
Glycodeoxycholic Acid: Calibration Standard Back-Calculated Concentrations in
Cholestyramine-Treated Human Saliva

| | Concentration (ng/mL) | | | | | | | | |
|---|---|---|---|---|---|---|---|---|---|
| | Std-1 | Std-2 | Std-3 | Std-4 | Std-5 | Std-6 | Std-7 | Std-8 | Std-9 |
| Analytical Run | 0.500 | 0.750 | 1.00 | 2.00 | 5.00 | 10.0 | 50.0 | 75.0 | 100 |
| B31 | 0.469 | 0.739 | 0.996 | 2.05 | 4.73 | 8.81 | 48.2 | 80.8 | 90.7 |
| | 0.501 | 0.783 | 1.05 | 2.15 | 5.02 | 10.6 | 46.7 | 82.5 | 101 |
| B32 | 0.502 | 0.725 | 0.985 | 2.17 | 4.87 | 9.63 | 49.9 | 76.0 | 97.3 |
| | 0.511 | 0.713 | 0.983 | 2.22 | 5.10 | 9.67 | 49.9 | 77.6 | 95.0 |
| B33 | 0.491 | 0.769 | 1.040 | 2.07 | 5.23 | 10.6 | 46.2 | 71.9 | 93.6 |
| | 0.480 | 0.742 | 0.981 | 2.19 | 4.78 | 11.1 | 45.4 | 72.4 | 103 |
| B34 | 0.477 | 0.756 | 1.05 | 2.09 | 4.88 | 8.69 | 46.3 | 69.9 | 100 |
| | 0.495 | 0.760 | 0.971 | 2.16 | 5.39 | 10.1 | 53.6 | 80.9 | 94.4 |
| B35 | 0.510 | 0.762 | 0.929 | 1.91 | 4.46 | 9.98 | 51.3 | 76.0 | 99.2 |
| | 0.499 | 0.794 | 0.994 | 1.97 | 4.59 | 10.5 | 48.9 | 85.6 | 103 |
| Mean | 0.494 | 0.754 | 0.998 | 2.10 | 4.91 | 9.97 | 48.6 | 77.4 | 97.7 |
| SD | 0.0141 | 0.0251 | 0.0385 | 0.100 | 0.286 | 0.786 | 2.60 | 5.08 | 4.20 |
| % CV | 2.9 | 3.3 | 3.9 | 4.8 | 5.8 | 7.9 | 5.3 | 6.6 | 4.3 |
| % Bias | −1.3 | 0.6 | −0.2 | 4.9 | −1.9 | −0.3 | −2.7 | 3.1 | −2.3 |
| n | 10 | 10 | 10 | 10 | 10 | 10 | 10 | 10 | 10 |

TABLE 59
Glycodeoxycholic Acid: Accuracy and Precision for Quality
Controls (Cholestyramine-Treated Human Saliva)

| | Concentration (ng/mL) | | | |
|---|---|---|---|---|
| Analytical | QC-LLOQ | QC-Low | QC-Mid | QC-High |
| Run | 0.500 | 1.50 | 8.00 | 80.0 |
| 31 | 0.566 | 1.54 | 8.05 | 79.5 |
| | 0.573 | 1.56 | 9.15 | 87.9 |
| | 0.592 | 1.57 | 9.10 | 77.2 |
| | 0.609 | 1.57 | 8.92 | 83.2 |
| Intra-Run Mean | 0.585 | 1.56 | 8.81 | 82.0 |
| Intra-Run SD | 0.0194 | 0.0141 | 0.513 | 4.67 |
| Intra-Run % CV | 3.3 | 0.9 | 5.8 | 5.7 |
| Intra-Run % Bias | 17.0 | 4.0 | 10.1 | 2.4 |
| n | 4 | 4 | 4 | 4 |
| 32 | 0.479 | 1.63 | 7.85 | 86.3 |
| | 0.457 | 1.67 | 7.86 | 76.3 |
| | 0.511 | 1.54 | 7.74 | 82.3 |
| | 0.500 | 1.66 | 8.34 | 78.6 |
| Intra-Run Mean | 0.487 | 1.63 | 7.95 | 80.9 |
| Intra-Run SD | 0.0239 | 0.0592 | 0.267 | 4.38 |
| Intra-Run % CV | 4.9 | 3.6 | 3.4 | 5.4 |
| Intra-Run % Bias | −2.7 | 8.3 | −0.7 | 1.1 |
| n | 4 | 4 | 4 | 4 |
| 33 | 0.535 | 1.74 | 8.92 | 86.7 |
| | 0.516 | 1.75 | 8.80 | 83.2 |
| | 0.559 | 1.86 | 8.75 | 77.6 |
| | 0.560 | 1.82 | 8.14 | 96.1 |
| Intra-Run Mean | 0.543 | 1.79 | 8.65 | 85.9 |
| Intra-Run SD | 0.0211 | 0.0574 | 0.349 | 7.76 |
| Intra-Run % CV | 3.9 | 3.2 | 4.0 | 9.0 |
| Intra-Run % Bias | 8.5 | 19.5 | 8.2 | 7.4 |
| n | 4 | 4 | 4 | 4 |
| Inter-Run Mean | 0.538 | 1.66 | 8.47 | 82.9 |
| Inter-Run SD | 0.0463 | 0.111 | 0.526 | 5.72 |
| Inter-Run % CV | 8.6 | 6.7 | 6.2 | 6.9 |
| Inter-Run % Bias | 7.6 | 10.6 | 5.9 | 3.6 |
| n | 12 | 12 | 12 | 12 |

TABLE 60
Glycodeoxycholic Acid: Frozen (−20° C.) Stability
in Cholestyramine-Treated Human Saliva

| | | Concentration (ng/mL) | | | |
|---|---|---|---|---|---|
| Storage | Analytical | QC-LLOQ | QC-Low | QC-Mid | QC-High |
| Period | Run | 0.500 | 1.50 | 8.00 | 80.0 |
| 1 Day | 33 | 0.488 | 1.55 | 7.61 | 73.6 |
| | | 0.519 | 1.72 | 7.21 | 75.5 |
| | | 0.500 | 1.68 | 7.43 | 76.0 |
| | | 0.472 | 1.62 | 8.14 | 78.9 |
| | Mean | 0.495 | 1.64 | 7.60 | 76.0 |
| | SD | 0.0198 | 0.0741 | 0.397 | 2.19 |
| | % CV | 4.0 | 4.5 | 5.2 | 2.9 |
| | % Bias | −1.1 | 9.5 | −5.0 | −5.0 |
| | n | 4 | 4 | 4 | 4 |
| 2 Days | 33 | 0.654^{a} | 1.47 | 7.91 | 74.0 |
| | | 0.589 | 1.46 | 7.87 | 73.3 |
| | | 0.551 | 1.48 | 8.54 | 75.6 |
| | | 0.599 | 1.50 | 8.49 | 77.7 |
| | Mean | 0.575 | 1.48 | 8.20 | 75.2 |
| | SD | 0.0339 | 0.0171 | 0.362 | 1.95 |
| | % CV | 5.9 | 1.2 | 4.4 | 2.6 |
| | % Bias | 15.0 | −1.5 | 2.5 | −6.1 |
| | n | 3 | 4 | 4 | 4 |

^{a}Failed to meet acceptance criteria (within ±30% from nominal concentration); excluded from statistics.

TABLE 61
Glycodeoxycholic Acid: Matrix Effects on Quantification
(Untreated Human Saliva)

| | Concentration (ng/mL) | | | | | |
|---|---|---|---|---|---|---|
| | Cholestyramine- | | | Untreated | | |
| | Treated Human Saliva | | | Human Saliva | | |
| | QC- | QC- | QC- | | QC- | QC- |
| | Low | Mid | High | Control | Low | High |
| Analytical Run | 1.50 | 8.00 | 80.0 | Blank | 1.50 | 80.0 |
| 34 | 1.64 | 8.93 | 75.7 | BQL | 1.55 | 77.2 |
| | 1.65 | 8.91 | 77.8 | BQL | 1.53 | 73.8 |
| | 1.67 | 8.45 | 79.1 | BQL | 1.45 | 76.5 |
| | NA | NA | NA | BQL | 1.46 | 77.3 |
| | NA | NA | NA | BQL | 1.45 | 77.3 |
| | NA | NA | NA | BQL | 1.42 | 75.7 |
| Mean | 1.65 | 8.76 | 77.5 | BQL | 1.48 | 76.3 |
| SD | 0.0153 | 0.272 | 1.72 | NA | 0.0513 | 1.38 |
| % CV | 0.9 | 3.1 | 2.2 | NA | 3.5 | 1.8 |
| % Bias | 10.2 | 9.5 | −3.1 | NA | −1.6 | −4.6 |
| n | 3 | 3 | 3 | 6 | 6 | 6 |

BQL Below quantitation limit (<0.500 ng/mL)
Note:
Bile acids are detected in some lots of untreated human saliva. The endogenous level for the lot used in this batch was BQL, therefore, the theoretical concentrations for QC-Low and QC-High in untreated human saliva are equal to the spiked concentrations (1.50 and 80.0 ng/mL).

TABLE 62
Glycodeoxycholic Acid: Long-Term (1 Month at −20° C. and
at −80° C.) Stability in Untreated Human Saliva

| | Concentration (ng/mL) | | | |
|---|---|---|---|---|
| | −20° C. Storage | | −80° C. Storage | |
| Analytical | QC-Low | QC-High | QC-Low | QC-High |
| Run | 1.50 | 80.0 | 1.50 | 80.0 |
| 35 | 1.42 | 79.2 | 1.28 | 77.8 |
| | 1.47 | 75.5 | 1.29 | 81.9 |
| | 1.48 | 75.9 | 1.35 | 80.0 |
| Mean | 1.46 | 76.9 | 1.31 | 79.9 |
| SD | 0.0321 | 2.03 | 0.0379 | 2.05 |
| % CV | 2.2 | 2.6 | 2.9 | 2.6 |
| % Bias | −2.9 | −3.9 | −12.9 | −0.1 |
| n | 3 | 3 | 3 | 3 |
| Fresh QCs (Batch 34) | | | | |
| Mean at Time Zero | 1.48 | 76.3 | 1.48 | 76.3 |
| Difference from | −1.6 | 0.7 | −11.7 | 4.7 |
| Fresh (%) | | | | |

TABLE 63
Glycodeoxycholic Acid: Batch Acceptance Quality
Controls (Cholestyramine-Treated Human Saliva)

| | Concentration (ng/mL) | | |
|---|---|---|---|
| Analytical | QC-Low | QC-Mid | QC-High |
| Run | 1.50 | 8.00 | 80.0 |
| 35 | 1.99^{a} | 8.31 | 77.6 |
| | 1.73 | 7.94 | 84.9 |
| | 1.83 | 9.84 | 83.3 |
| Mean | 1.78 | 8.70 | 81.9 |
| SD | 0.0707 | 1.01 | 3.84 |
| % CV | 4.0 | 11.6 | 4.7 |
| % Bias | 18.7 | 8.7 | 2.4 |
| n | 2 | 3 | 3 |

^{a}Failed to meet acceptance criteria (within ±30% from nominal concentration); excluded from statistics.

TABLE 64
Lithocholic Acid: Calibration Standard Back-Calculated Concentrations in Cholestyramine-Treated Human
Saliva

| | Concentration (ng/mL) | | | | | | | | |
|---|---|---|---|---|---|---|---|---|---|
| | Std-1 | Std-2 | Std-3 | Std-4 | Std-5 | Std-6 | Std-7 | Std-8 | Std-9 |
| Analytical Run | 5.00 | 7.50 | 10.0 | 20.0 | 50.0 | 100 | 500 | 750 | 1000 |
| B31 | NR | NR | 10.70 | 24.5 | 42.6 | 88.5 | 475 | 884 | 999 |
| | NR | NR | 9.37 | 18.3 | 42.3 | 95.8 | 461 | 880 | 1090 |
| B32 | NR | NR | 11.9 | 23.2 | 47.8 | 91.3 | 480 | 858 | 1100 |
| | NR | NR | 7.81 | 19.1 | 50.2 | 84.5 | 500 | 754 | 990 |
| B33 | NR | NR | 11.3 | 20.4 | 47.0 | 114 | 462 | 708 | 1060 |
| | NR | NR | 9.53 | 16.4 | 45.9 | 111 | 396 | 824 | 1140 |
| B34 | NR | NR | 9.73 | 21.8 | 51.2 | 97.5 | 526 | 748 | 1000 |
| | NR | NR | 9.62 | 19.8 | 57.4 | 94.8 | 488 | 674 | 964 |
| B36 | NR | NR | 9.41 | 23.3 | 58.2 | 115 | 513 | 762 | 898 |
| | NR | NR | 9.57 | 18.5 | 52.1 | 104 | 458 | 686 | 840 |
| Mean | NA | NA | 9.89 | 20.5 | 49.5 | 100 | 476 | 778 | 1010 |
| SD | NA | NA | 1.15 | 2.60 | 5.48 | 10.8 | 36.1 | 78.9 | 93.1 |
| % CV | NA | NA | 11.6 | 12.7 | 11.1 | 10.9 | 7.6 | 10.1 | 9.2 |
| % Bias | NA | NA | −1.1 | 2.7 | −1.1 | −0.4 | −4.8 | 3.7 | 0.8 |
| n | NA | NA | 10 | 10 | 10 | 10 | 10 | 10 | 10 |

NR: Not reported: calibration standard was not used: curve was truncated

TABLE 65
Lithocholic Acid: Accuracy and Precision for Quality
Controls (Cholestyramine-Treated Human Saliva)

| | Concentration (ng/mL) | | | |
|---|---|---|---|---|
| Analytical | QC-LLOQ^{a} | QC-Low | QC-Mid | QC-High |
| Run | 5.00 | 15.0 | 80.0 | 800 |
| 31 | NA | 21.5^{b} | 106^{b} | 834 |
| | NA | 25.2^{b} | 104^{b} | 951 |
| | NA | 19.2 | 95.7 | 757 |
| | NA | 17.3 | 102 | 892 |
| Intra-Run Mean | NA | 18.3 | 98.9 | 859 |
| Intra-Run SD | NA | 1.34 | 4.45 | 82.8 |
| Intra-Run % CV | NA | 7.4 | 4.5 | 9.6 |
| Intra-Run % Bias | NA | 21.7 | 23.6 | 7.3 |
| n | NA | 2 | 2 | 4 |
| 32 | NA | 19.0 | 82.1 | 1110^{b} |
| | NA | 18.9 | 85.3 | 910 |
| | NA | 17.9 | 89.4 | 981 |
| | NA | 20.5^{b} | 93.4 | 989 |
| Intra-Run Mean | NA | 18.6 | 87.6 | 960 |
| Intra-Run SD | NA | 0.608 | 4.91 | 43.5 |
| Intra-Run % CV | NA | 3.3 | 5.6 | 4.5 |
| Intra-Run % Bias | NA | 24.0 | 9.4 | 20.0 |
| n | NA | 3 | 4 | 3 |
| 33 | NA | 17.4 | 69.1 | 728 |
| | NA | 16.0 | 74.8 | 657 |
| | NA | 15.2 | 89.1 | 799 |
| | NA | 15.8 | 74.6 | 1010 |
| Intra-Run Mean | NA | 16.1 | 76.9 | 799 |
| Intra-Run SD | NA | 0.931 | 8.55 | 152 |
| Intra-Run % CV | NA | 5.8 | 11.1 | 19.1 |
| Intra-Run % Bias | NA | 7.3 | −3.9 | −0.2 |
| n | NA | 4 | 4 | 4 |
| Inter-Run Mean | NA | 17.4 | 85.6 | 864 |
| Inter-Run SD | NA | 1.49 | 10.4 | 118 |
| Inter-Run % CV | NA | 8.5 | 12.2 | 13.6 |
| Inter-Run % Bias | NA | 16.1 | 6.9 | 8.0 |
| n | NA | 9 | 10 | 11 |

^{a}Due to truncated curve, QC was not included.
^{b}Failed to meet acceptance criteria (within ±30% from nominal concentration); excluded from statistics.

TABLE 66
Lithocholic Acid: Frozen (−20° C.) Stability
in Cholestyramine-Treated Human Saliva

| | | Concentration (ng/mL) | | | |
|---|---|---|---|---|---|
| Storage | Analytical | QC-LLOQ^{a} | QC-Low | QC-Mid | QC-High |
| Period | Run | 5.00 | 15.0 | 80.0 | 800 |
| 1 Day | 33 | NA | 16.9 | 77.6 | 746 |
| | | NA | 17.0 | 83.0 | 833 |
| | | NA | 15.9 | 81.3 | 676 |
| | | NA | 16.4 | 83.8 | 655 |
| | Mean | NA | 16.6 | 81.4 | 728 |
| | SD | NA | 0.507 | 2.75 | 80.4 |
| | % CV | NA | 3.1 | 3.4 | 11.0 |
| | % Bias | NA | 10.3 | 1.8 | −9.1 |
| | n | NA | 4 | 4 | 4 |
| 2 Days | 33 | NA | 17.6 | 96.5 | 788 |
| | | NA | 13.7 | 76.5 | 722 |
| | | NA | 15.6 | 73.5 | 947 |
| | | NA | 16.0 | 101 | 794 |
| | Mean | NA | 15.7 | 86.9 | 813 |
| | SD | NA | 1.60 | 13.9 | 95.3 |
| | % CV | NA | 10.2 | 16.0 | 11.7 |
| | % Bias | NA | 4.8 | 8.6 | 1.6 |
| | n | NA | 4 | 4 | 4 |

^{a}Due to truncated curve, QC was not included.

TABLE 67
Lithocholic Acid: Matrix Effects on Quantification
(Untreated Human Saliva)

| | Concentration (ng/mL) | | | | | |
|---|---|---|---|---|---|---|
| | Cholestyramine- | | | Untreated | | |
| | Treated Human Saliva | | | Human Saliva | | |
| | QC- | QC- | QC- | | QC- | QC- |
| | Low | Mid | High | Control | Low | High |
| Analytical Run | 15.0 | 80.0 | 800 | Blank | 15.0 | 800 |
| 34 | 16.8 | 94.6 | 852 | BQL | 13.2 | 832 |
| | 15.9 | 87.8 | 797 | BQL | 12.8 | 761 |
| | 16.8 | 84.8 | 807 | BQL | 12.3 | 748 |
| | NA | NA | NA | BQL | 12.7 | 793 |
| | NA | NA | NA | BQL | 12.7 | 826 |
| | NA | NA | NA | BQL | 13.4 | 798 |
| Mean | 16.5 | 89.1 | 819 | BQL | 12.9 | 793 |
| SD | 0.520 | 5.02 | 29.3 | NA | 0.394 | 33.7 |
| % CV | 3.1 | 5.6 | 3.6 | NA | 3.1 | 4.3 |
| % Bias | 10.0 | 11.3 | 2.3 | NA | −14.3 | −0.9 |
| n | 3 | 3 | 3 | 6 | 6 | 6 |

BQL Below quantitation limit (<10.0 ng/mL)
Note:
Bile acids are detected in some lots of untreated human saliva. The endogenous level for the lot used in this batch was BQL, therefore, the theoretical concentrations for QC-Low and QC-High in untreated human saliva are equal to the spiked concentrations (15.0 and 800 ng/mL).

TABLE 68
Lithocholic Acid: Long-Tenn (1 Month at −20° C. and at −80°
C.) Stability in Untreated Human Saliva

| | Concentration (ng/mL) | | | |
|---|---|---|---|---|
| | −20° C. Storage | | −80° C. Storage | |
| Analytical | QC-Low | QC-High | QC-Low | QC-High |
| Run | 15.0 | 800 | 15.0 | 800 |
| 36 | 16.7 | 822 | 9.13^{a} | 708 |
| | 17.5 | 782 | 7.70^{a} | 670 |
| | 18.2 | 794 | 9.96^{a} | 683 |
| Mean | 17.5 | 799 | 8.93 | 687 |
| SD | 0.751 | 20.5 | 1.14 | 19.3 |
| % CV | 4.3 | 2.6 | 12.8 | 2.8 |
| % Bias | 16.4 | −0.1 | −40.5 | −14.1 |
| n | 3 | 3 | 3 | 3 |
| Fresh QCs (Batch 34) | | | | |
| Mean at Time Zero | 12.9 | 793 | 12.9 | 793 |
| Difference from | 35.4 | 0.8 | −30.8 | −13.4 |
| Fresh (%) | | | | |

^{a}Failed to meet acceptance criteria (within ±30% from nominal concentration).

TABLE 69
Lithocholic Acid: Batch Acceptance Quality Controls
(Cholestyramine-Treated Human Saliva)

| | Concentration (ng/mL) | | |
|---|---|---|---|
| Analytical | QC-Low | QC-Mid | QC-High |
| Run | 15.0 | 80.0 | 800 |
| 36 | 19.1 | 95.5 | 876 |
| | 17.0 | 95.1 | 890 |
| | 15.3 | 94.6 | 790 |
| Mean | 17.1 | 95.1 | 852 |
| SD | 1.90 | 0.451 | 54.1 |
| % CV | 11.1 | 0.5 | 6.4 |
| % Bias | 14.2 | 18.8 | 6.5 |
| n | 3 | 3 | 3 |

TABLE 70
Taurodeoxycholic Acid: Calibration Standard Back-Calculated
Concentrations in Cholestyramine-Treated Human Saliva

| | Concentration (ng/mL) | | | | | | | | |
|---|---|---|---|---|---|---|---|---|---|
| | Std-1 | Std-2 | Std-3 | Std-4 | Std-5 | Std-6 | Std-7 | Std-8 | Std-9 |
| Analytical Run | 0.500 | 0.750 | 1.00 | 2.00 | 5.00 | 10.0 | 50.0 | 75.0 | 100 |
| B31 | 0.493 | 0.755 | 0.978 | 1.97 | 4.28 | 9.41 | 49.5 | 83.9 | 94.9 |
| | 0.489 | 0.780 | 1.06 | 2.08 | 4.92 | 9.72 | 48.8 | 83.7 | 102 |
| B32 | 0.499 | 0.713 | 1.02 | 2.20 | 4.90 | 9.40 | 49.2 | 76.3 | 96.4 |
| | 0.498 | 0.751 | 0.970 | 2.15 | 5.16 | 9.58 | 50.2 | 77.5 | 96.9 |
| B33 | 0.509 | 0.732 | 0.974 | 2.06 | 4.93 | 10.9 | 46.5 | 72.9 | 95.8 |
| | 0.505 | 0.755 | 0.966 | 1.98 | 5.02 | 11.3 | 45.6 | 74.3 | 106 |
| B34 | 0.508 | 0.721 | 0.991 | 1.92 | 5.09 | 9.49 | 48.3 | 74.1 | 106 |
| | 0.533 | 0.732 | 0.944 | 1.95 | 5.63 | 9.34 | 50.7 | 78.8 | 100 |
| B35 | 0.498 | 0.778 | 0.948 | 1.92 | 4.41 | 9.86 | 51.1 | 77.7 | 98.3 |
| | 0.508 | 0.775 | 0.978 | 2.05 | 4.59 | 10.20 | 50.0 | 86.0 | 102 |
| Mean | 0.504 | 0.749 | 0.983 | 2.03 | 4.89 | 9.92 | 49.0 | 78.5 | 99.8 |
| SD | 0.0122 | 0.0241 | 0.0345 | 0.0965 | 0.390 | 0.679 | 1.78 | 4.56 | 4.07 |
| % CV | 2.4 | 3.2 | 3.5 | 4.8 | 8.0 | 6.8 | 3.6 | 5.8 | 4.1 |
| % Bias | 0.8 | −0.1 | −1.7 | 1.4 | −2.1 | −0.8 | −2.0 | 4.7 | −0.2 |
| n | 10 | 10 | 10 | 10 | 10 | 10 | 10 | 10 | 10 |

TABLE 71
Taurodeoxycholic Acid: Accuracy and Precision for Quality
Controls (Cholestyramine-Treated Human Saliva)

| | Concentration (ng/mL) | | | |
|---|---|---|---|---|
| Analytical | QC-LLOQ | QC-Low | QC-Mid | QC-High |
| Run | 0.500 | 1.50 | 8.00 | 80.0 |
| 31 | 0.610 | 1.55 | 8.98 | 83.5 |
| | 0.584 | 1.60 | 9.08 | 88.0 |
| | 0.665^{a} | 1.63 | 9.15 | 78.6 |
| | 0.613 | 1.43 | 8.96 | 84.7 |
| Intra-Run Mean | 0.602 | 1.55 | 9.04 | 83.7 |
| Intra-Run SD | 0.0159 | 0.0881 | 0.0888 | 3.90 |
| Intra-Run % CV | 2.6 | 5.7 | 1.0 | 4.7 |
| Intra-Run % Bias | 20.5 | 3.5 | 13.0 | 4.6 |
| n | 3 | 4 | 4 | 4 |
| 32 | 0.502 | 1.70 | 7.94 | 88.8 |
| | 0.478 | 1.68 | 7.85 | 77.4 |
| | 0.520 | 1.63 | 7.75 | 85.7 |
| | 0.523 | 1.71 | 8.38 | 83.1 |
| Intra-Run Mean | 0.506 | 1.68 | 7.98 | 83.8 |
| Intra-Run SD | 0.0207 | 0.0356 | 0.278 | 4.83 |
| Intra-Run % CV | 4.1 | 2.1 | 3.5 | 5.8 |
| Intra-Run % Bias | 1.2 | 12.0 | −0.2 | 4.7 |
| n | 4 | 4 | 4 | 4 |
| 33 | 0.534 | 1.64 | 8.08 | 88.4 |
| | 0.541 | 1.65 | 7.96 | 85.4 |
| | 0.576 | 1.73 | 7.89 | 76.6 |
| | 0.599 | 1.65 | 7.88 | 99.3 |
| Intra-Run Mean | 0.563 | 1.67 | 7.95 | 87.4 |
| Intra-Run SD | 0.0305 | 0.0419 | 0.0922 | 9.37 |
| Intra-Run % CV | 5.4 | 2.5 | 1.2 | 10.7 |
| Intra-Run % Bias | 12.5 | 11.2 | −0.6 | 9.3 |
| n | 4 | 4 | 4 | 4 |
| Inter-Run Mean | 0.553 | 1.63 | 8.33 | 85.0 |
| Inter-Run SD | 0.0460 | 0.0808 | 0.554 | 6.14 |
| Inter-Run % CV | 8.3 | 4.9 | 6.6 | 7.2 |
| Inter-Run % Bias | 10.5 | 8.9 | 4.1 | 6.2 |
| n | 11 | 12 | 12 | 12 |

^{a}Failed to meet acceptance criteria (within ±30% from nominal concentration); excluded from statistics.

TABLE 72
Taurodeoxycholic Acid: Frozen (−20° C.) Stability
in Cholestyramine-Treated Human Saliva

| | | Concentration (ng/mL) | | | |
|---|---|---|---|---|---|
| Storage | Analytical | QC-LLOQ | QC-Low | QC-Mid | QC-High |
| Period | Run | 0.500 | 1.50 | 8.00 | 80.0 |
| 1 Day | 33 | 0.536 | 1.52 | 7.81 | 76.9 |
| | | 0.560 | 1.63 | 7.77 | 78.5 |
| | | 0.539 | 1.68 | 7.71 | 78.6 |
| | | 0.570 | 1.59 | 7.90 | 82.0 |
| | Mean | 0.551 | 1.61 | 7.80 | 79.0 |
| | SD | 0.0164 | 0.0676 | 0.0797 | 2.15 |
| | % CV | 3.0 | 4.2 | 1.0 | 2.7 |
| | % Bias | 10.3 | 7.0 | −2.5 | −1.3 |
| | n | 4 | 4 | 4 | 4 |
| 2 Days | 33 | 0.596 | 1.38 | 7.19 | 73.7 |
| | | 0.592 | 1.35 | 8.02 | 76.5 |
| | | 0.555 | 1.40 | 8.13 | 76.2 |
| | | 0.601 | 1.42 | 7.72 | 79.5 |
| | Mean | 0.586 | 1.39 | 7.77 | 76.5 |
| | SD | 0.0210 | 0.0299 | 0.421 | 2.38 |
| | % CV | 3.6 | 2.2 | 5.4 | 3.1 |
| | % Bias | 17.2 | −7.5 | −2.9 | −4.4 |
| | n | 4 | 4 | 4 | 4 |

TABLE 73
Taurodeoxycholic Acid: Matrix Effects on Quantification
(Untreated Human Saliva)

| | Concentration (ng/mL) | | | | | |
|---|---|---|---|---|---|---|
| | Cholestyramine- | | | Untreated | | |
| | Treated Human Saliva | | | Human Saliva | | |
| | QC- | QC- | QC- | | QC- | QC- |
| | Low | Mid | High | Control | Low | High |
| Analytical Run | 1.50 | 8.00 | 80.0 | Blank | 1.50 | 80.0 |
| 34 | 1.45 | 8.14 | 78.8 | BQL | 1.30 | 82.4 |
| | 1.50 | 7.94 | 78.2 | BQL | 1.25 | 77.7 |
| | 1.52 | 7.95 | 80.5 | BQL | 1.35 | 79.8 |
| | NA | NA | NA | BQL | 1.28 | 80.1 |
| | NA | NA | NA | BQL | 1.25 | 81.8 |
| | NA | NA | NA | BQL | 1.30 | 82.3 |
| Mean | 1.49 | 8.01 | 79.2 | BQL | 1.29 | 80.7 |
| SD | 0.0361 | 0.113 | 1.19 | NA | 0.0376 | 1.83 |
| % CV | 2.4 | 1.4 | 1.5 | NA | 2.9 | 2.3 |
| % Bias | −0.7 | 0.1 | −1.0 | NA | −14.1 | 0.9 |
| n | 3 | 3 | 3 | 6 | 6 | 6 |

BQL Below quantitation limit (<0.500 ng/mL)
Note:
Bile acids are detected in some lots of untreated human saliva. The endogenous level for the lot used in this batch was BQL, therefore, the theoretical concentrations for QC-Low and QC-High in untreated human saliva are equal to the spiked concentrations (1.50 and 80.0 ng/mL).

TABLE 74
Taurodeoxycholic Acid: Long-Term (1 Month at −20° C. and
at −80° C.) Stability in Untreated Human Saliva

| | Concentration (ng/mL) | | | |
|---|---|---|---|---|
| | −20° C. Storage | | −80° C. Storage | |
| Analytical | QC-Low | QC-High | QC-Low | QC-High |
| Run | 1.50 | 80.0 | 1.50 | 80.0 |
| 35 | 1.26 | 76.0 | 1.20 | 73.9 |
| | 1.37 | 73.7 | 1.18 | 80.2 |
| | 1.42 | 79.0 | 1.32 | 79.4 |
| Mean | 1.35 | 76.2 | 1.23 | 77.8 |
| SD | 0.0819 | 2.66 | 0.0757 | 3.43 |
| % CV | 6.1 | 3.5 | 6.1 | 4.4 |
| % Bias | −10.0 | −4.7 | −17.8 | −2.7 |
| n | 3 | 3 | 3 | 3 |
| Fresh QCs (Batch 34) | | | | |
| Mean at Time Zero | 1.29 | 80.7 | 1.29 | 80.7 |
| Difference from | 4.7 | −5.5 | −4.4 | −3.6 |
| Fresh (%) | | | | |

TABLE 75
Taurodeoxycholic Acid: Batch Acceptance Quality
Controls (Cholestyramine-Treated Human Saliva)

| | Concentration (ng/mL) | | |
|---|---|---|---|
| Analytical | QC-Low | QC-Mid | QC-High |
| Run | 1.50 | 8.00 | 80.0 |
| 35 | 1.92 | 7.99 | 77.4 |
| | 1.73 | 8.06 | 89.3 |
| | 1.79 | 9.53 | 85.9 |
| Mean | 1.81 | 8.53 | 84.2 |
| SD | 0.0971 | 0.870 | 6.13 |
| % CV | 5.4 | 10.2 | 7.3 |
| % Bias | 20.9 | 6.6 | 5.3 |
| n | 3 | 3 | 3 |

TABLE 76
Taurocholic Acid: Calibration Standard Back-Calculated Concentrations in Cholestyramine-Treated Human
Saliva

| | Concentration (ng/mL) | | | | | | | | |
|---|---|---|---|---|---|---|---|---|---|
| | Std-1 | Std-2 | Std-3 | Std-4 | Std-5 | Std-6 | Std-7 | Std-8 | Std-9 |
| Analytical Run | 0.500 | 0.750 | 1.00 | 2.00 | 5.00 | 10.0 | 50.0 | 75.0 | 100 |
| B31 | 0.492 | 0.737 | 0.967 | 1.91 | 4.53 | 9.51 | 51.3 | 82.9 | 94.4 |
| | 0.522 | 0.763 | 1.02 | 2.02 | 4.81 | 10.4 | 47.8 | 84.9 | 100 |
| B32 | 0.493 | 0.699 | 1.040 | 2.19 | 4.93 | 9.79 | 48.5 | 73.3 | 96.1 |
| | 0.501 | 0.735 | 1.01 | 2.20 | 5.09 | 9.77 | 51.1 | 77.6 | 93.2 |
| B33 | 0.478 | 0.709 | 0.987 | 2.01 | 4.90 | 11.0 | 46.9 | 72.4 | 96.3 |
| | 0.529 | 0.766 | 0.991 | 2.05 | 5.08 | 11.5 | 46.2 | 72.0 | 101 |
| B34 | 0.491 | 0.735 | 1.00 | 2.00 | 5.25 | 9.46 | 49.6 | 74.1 | 103 |
| | 0.523 | 0.730 | 0.994 | 1.98 | 5.45 | 9.18 | 50.3 | 76.1 | 99.2 |
| B35 | 0.521 | 0.789 | 0.970 | 1.92 | 4.58 | 9.97 | 52.3 | 77.7 | 99.5 |
| | 0.500 | 0.741 | 0.947 | 1.97 | 4.67 | 10.40 | 48.1 | 83.0 | 102 |
| Mean | 0.505 | 0.740 | 0.992 | 2.03 | 4.93 | 10.1 | 49.2 | 77.4 | 98.5 |
| SD | 0.0174 | 0.0267 | 0.0276 | 0.0992 | 0.296 | 0.728 | 2.03 | 4.73 | 3.30 |
| % CV | 3.4 | 3.6 | 2.8 | 4.9 | 6.0 | 7.2 | 4.1 | 6.1 | 3.4 |
| % Bias | 1.0 | −1.3 | −0.8 | 1.3 | −1.4 | 1.0 | −1.6 | 3.2 | −1.5 |
| n | 10 | 10 | 10 | 10 | 10 | 10 | 10 | 10 | 10 |

TABLE 77
Taurocholic Acid: Accuracy and Precision for Quality
Controls (Cholestyramine-Treated Human Saliva)

| | Concentration (ng/mL) | | | |
|---|---|---|---|---|
| Analytical | QC-LLOQ | QC-Low | QC-Mid | QC-High |
| Run | 0.500 | 1.50 | 8.00 | 80.0 |
| 31 | 0.581 | 1.42 | 8.32 | 81.4 |
| | 0.589 | 1.46 | 8.38 | 88.2 |
| | 0.628 | 1.49 | 8.00 | 79.9 |
| | 0.587 | 1.44 | 8.15 | 82.2 |
| Intra-Run Mean | 0.596 | 1.45 | 8.21 | 82.9 |
| Intra-Run SD | 0.0214 | 0.0299 | 0.172 | 3.64 |
| Intra-Run % CV | 3.6 | 2.1 | 2.1 | 4.4 |
| Intra-Run % Bias | 19.3 | −3.2 | 2.7 | 3.7 |
| n | 4 | 4 | 4 | 4 |
| 32 | 0.482 | 1.69 | 7.75 | 86.3 |
| | 0.445 | 1.65 | 7.93 | 77.4 |
| | 0.501 | 1.55 | 7.63 | 85.4 |
| | 0.513 | 1.68 | 8.12 | 80.5 |
| Intra-Run Mean | 0.485 | 1.64 | 7.86 | 82.4 |
| Intra-Run SD | 0.0297 | 0.0640 | 0.214 | 4.20 |
| Intra-Run % CV | 6.1 | 3.9 | 2.7 | 5.1 |
| Intra-Run % Bias | −3.0 | 9.5 | −1.8 | 3.0 |
| n | 4 | 4 | 4 | 4 |
| 33 | 0.524 | 1.55 | 7.53 | 82.2 |
| | 0.516 | 1.60 | 7.44 | 78.2 |
| | 0.535 | 1.58 | 7.52 | 73.9 |
| | 0.536 | 1.56 | 7.41 | 88.6 |
| Intra-Run Mean | 0.528 | 1.57 | 7.48 | 80.7 |
| Intra-Run SD | 0.00954 | 0.0222 | 0.0592 | 6.25 |
| Intra-Run % CV | 1.8 | 1.4 | 0.8 | 7.7 |
| Intra-Run % Bias | 5.6 | 4.8 | −6.6 | 0.9 |
| n | 4 | 4 | 4 | 4 |
| Inter-Run Mean | 0.536 | 1.56 | 7.85 | 82.0 |
| Inter-Run SD | 0.0517 | 0.0906 | 0.347 | 4.48 |
| Inter-Run % CV | 9.6 | 5.8 | 4.4 | 5.5 |
| Inter-Run % Bias | 7.3 | 3.7 | −1.9 | 2.5 |
| n | 12 | 12 | 12 | 12 |

TABLE 78
Taurocholic Acid: Frozen (−20° C.) Stability
in Cholestyramine-Treated Human Saliva

| | | Concentration (ng/mL) | | | |
|---|---|---|---|---|---|
| Storage | Analytical | QC-LLOQ | QC-Low | QC-Mid | QC-High |
| Period | Run | 0.500 | 1.50 | 8.00 | 80.0 |
| 1 Day | 33 | 0.491 | 1.45 | 7.51 | 72.3 |
| | | 0.498 | 1.55 | 7.18 | 74.5 |
| | | 0.481 | 1.59 | 7.32 | 74.9 |
| | | 0.509 | 1.48 | 7.29 | 77.2 |
| | Mean | 0.495 | 1.52 | 7.33 | 74.7 |
| | SD | 0.0118 | 0.0640 | 0.137 | 2.01 |
| | % CV | 2.4 | 4.2 | 1.9 | 2.7 |
| | % Bias | −1.1 | 1.2 | −8.4 | −6.6 |
| | n | 4 | 4 | 4 | 4 |
| 2 Days | 33 | 0.538 | 1.24 | 6.98 | 73.1 |
| | | 0.563 | 1.28 | 7.47 | 75.1 |
| | | 0.537 | 1.35 | 7.55 | 73.8 |
| | | 0.585 | 1.37 | 7.85 | 76.6 |
| | Mean | 0.556 | 1.31 | 7.46 | 74.7 |
| | SD | 0.0229 | 0.0606 | 0.361 | 1.54 |
| | % CV | 4.1 | 4.6 | 4.8 | 2.1 |
| | % Bias | 11.2 | −12.7 | −6.7 | −6.7 |
| | n | 4 | 4 | 4 | 4 |

TABLE 79
Taurocholic Acid: Matrix Effects on Quantification
(Untreated Human Saliva)

| | Concentration (ng/mL) | | | | | |
|---|---|---|---|---|---|---|
| | Cholestyramine- | | | Untreated | | |
| | Treated Human Saliva | | | Human Saliva | | |
| | QC- | QC- | QC- | | QC- | QC- |
| | Low | Mid | High | Control | Low | High |
| Analytical Run | 1.50 | 8.00 | 80.0 | Blank | 1.50 | 80.0 |
| 34 | 1.55 | 8.81 | 81.7 | BQL | 1.37 | 81.2 |
| | 1.49 | 7.99 | 80.3 | BQL | 1.37 | 80.2 |
| | 1.55 | 7.98 | 80.3 | BQL | 1.35 | 82.8 |
| | NA | NA | NA | BQL | 1.33 | 81.6 |
| | NA | NA | NA | BQL | 1.29 | 80.2 |
| | NA | NA | NA | BQL | 1.29 | 82.2 |
| Mean | 1.53 | 8.26 | 80.8 | BQL | 1.33 | 81.4 |
| SD | 0.0346 | 0.476 | 0.808 | NA | 0.0367 | 1.05 |
| % CV | 2.3 | 5.8 | 1.0 | NA | 2.8 | 1.3 |
| % Bias | 2.0 | 3.3 | 1.0 | NA | −11.1 | 1.7 |
| n | 3 | 3 | 3 | 6 | 6 | 6 |

BQL Below quantitation limit (<0.500 ng/mL)
Note:
Bile acids are detected in some lots of untreated human saliva. The endogenous level for the lot used in this batch was BQL, therefore, the theoretical concentrations for QC-Low and QC-High in untreated human saliva are equal to the spiked concentrations (1.50 and 80.0 ng/mL).

TABLE 80
Taurocholic Acid: Long-Term (1 Month at −20° C. and at −80°
C.) Stability in Untreated Human Saliva

| | Concentration (ng/mL) | | | |
|---|---|---|---|---|
| | −20° C. Storage | | −80° C. Storage | |
| Analytical | QC-Low | QC-High | QC-Low | QC-High |
| Run | 1.50 | 80.0 | 1.50 | 80.0 |
| 35 | 1.18 | 79.7 | 1.13 | 75.4 |
| | 1.24 | 75.1 | 1.13 | 81.2 |
| | 1.28 | 78.3 | 1.20 | 77.8 |
| Mean | 1.23 | 77.7 | 1.15 | 78.1 |
| SD | 0.0503 | 2.36 | 0.0404 | 2.914 |
| % CV | 4.1 | 3.0 | 3.5 | 3.7 |
| % Bias | −17.8 | −2.9 | −23.1 | −2.3 |
| n | 3 | 3 | 3 | 3 |
| Fresh QCs (Batch 34) | | | | |
| Mean | 1.33 | 81.4 | 1.33 | 81.4 |
| Difference from | −7.3 | −4.5 | −13.3 | −4.0 |
| Fresh (%) | | | | |

TABLE 81
Taurocholic Acid: Batch Acceptance Quality Controls
(Cholestyramine-Treated Human Saliva)

| | Concentration (ng/mL) | | |
|---|---|---|---|
| Analytical | QC-Low | QC-Mid | QC-High |
| Run | 1.50 | 8.00 | 80.0 |
| 35 | 1.90 | 7.64 | 74.0 |
| | 1.65 | 7.53 | 82.5 |
| | 1.64 | 8.73 | 76.3 |
| Mean | 1.73 | 7.97 | 77.6 |
| SD | 0.147 | 0.663 | 4.40 |
| % CV | 8.5 | 8.3 | 5.7 |
| % Bias | 15.3 | −0.4 | −3.0 |
| n | 3 | 3 | 3 |

TABLE 82
Glycochenodeoxycholic Acid: Calibration Standard Back-Calculated Concentrations in Cholestyramine-
Treated Human Saliva

| | Concentration (ng/mL) | | | | | | | | |
|---|---|---|---|---|---|---|---|---|---|
| | Std-1 | Std-2 | Std-3 | Std-4 | Std-5 | Std-6 | Std-7 | Std-8 | Std-9 |
| Analytical Run | 0.500 | 0.750 | 1.00 | 2.00 | 5.00 | 10.0 | 50.0 | 75.0 | 100 |
| B31 | 0.518 | 0.746 | 0.984 | 1.87 | 4.54 | 9.45 | 49.3 | 81.9 | 95.8 |
| | 0.483 | 0.775 | 1.02 | 2.06 | 4.75 | 10.2 | 48.3 | 84.8 | 104 |
| B32 | 0.508 | 0.730 | 1.01 | 2.18 | 4.93 | 9.56 | 49.2 | 76.5 | 97.0 |
| | 0.485 | 0.744 | 0.986 | 2.17 | 5.10 | 9.58 | 50.3 | 77.1 | 95.3 |
| B33 | 0.513 | 0.686 | 0.993 | 2.08 | 4.76 | 11.0 | 46.9 | 72.7 | 96.5 |
| | 0.514 | 0.734 | 1.00 | 2.03 | 4.99 | 11.3 | 45.4 | 74.8 | 104 |
| B34 | 0.477 | 0.748 | 1.01 | 1.98 | 5.16 | 9.23 | 47.9 | 72.9 | 101 |
| | 0.492 | 0.765 | 1.07 | 2.03 | 5.67 | 9.24 | 50.3 | 75.3 | 99.4 |
| B35 | 0.508 | 0.814 | 0.951 | 2.03 | 4.40 | 9.84 | 52.1 | 76.7 | 95.4 |
| | 0.499 | 0.753 | 0.936 | 1.95 | 4.71 | 10.4 | 49.3 | 85.8 | 102 |
| Mean | 0.500 | 0.750 | 0.996 | 2.04 | 4.90 | 9.98 | 48.9 | 77.9 | 99.0 |
| SD | 0.0146 | 0.0329 | 0.0370 | 0.0939 | 0.360 | 0.727 | 1.89 | 4.70 | 3.50 |
| % CV | 2.9 | 4.4 | 3.7 | 4.6 | 7.3 | 7.3 | 3.9 | 6.0 | 3.5 |
| % Bias | −0.1 | −0.1 | −0.4 | 1.9 | −2.0 | −0.2 | −2.2 | 3.8 | −1.0 |
| n | 10 | 10 | 10 | 10 | 10 | 10 | 10 | 10 | 10 |

TABLE 83
Glycochenodeoxycholic Acid: Accuracy and Precision for
Quality Controls (Cholestyramine-Treated Human Saliva)

| | Concentration (ng/mL) | | | |
|---|---|---|---|---|
| Analytical | QC-LLOQ | QC-Low | QC-Mid | QC-High |
| Run | 0.500 | 1.50 | 8.00 | 80.0 |
| 31 | 0.647 | 1.57 | 8.30 | 84.0 |
| | 0.641 | 1.64 | 8.65 | 90.8 |
| | 0.701^{a} | 1.59 | 8.65 | 81.6 |
| | 0.645 | 1.57 | 8.67 | 86.7 |
| Intra-Run Mean | 0.644 | 1.59 | 8.57 | 85.8 |
| Intra-Run SD | 0.00306 | 0.0330 | 0.179 | 3.94 |
| Intra-Run % CV | 0.5 | 2.1 | 2.1 | 4.6 |
| Intra-Run % Bias | 28.9 | 6.2 | 7.1 | 7.2 |
| n | 3 | 4 | 4 | 4 |
| 32 | 0.519 | 1.66 | 7.77 | 86.0 |
| | 0.466 | 1.67 | 7.82 | 76.5 |
| | 0.500 | 1.58 | 7.67 | 83.1 |
| | 0.485 | 1.68 | 8.06 | 78.6 |
| Intra-Run Mean | 0.493 | 1.65 | 7.83 | 81.1 |
| Intra-Run SD | 0.0225 | 0.0457 | 0.166 | 4.30 |
| Intra-Run % CV | 4.6 | 2.8 | 2.1 | 5.3 |
| Intra-Run % Bias | −1.5 | 9.8 | −2.1 | 1.3 |
| n | 4 | 4 | 4 | 4 |
| 33 | 0.555 | 1.68 | 8.18 | 87.1 |
| | 0.594 | 1.68 | 8.18 | 85.1 |
| | 0.586 | 1.72 | 8.20 | 78.2 |
| | 0.594 | 1.65 | 8.09 | 99.4 |
| Intra-Run Mean | 0.582 | 1.68 | 8.16 | 87.5 |
| Intra-Run SD | 0.0186 | 0.0287 | 0.0492 | 8.83 |
| Intra-Run % CV | 3.2 | 1.7 | 0.6 | 10.1 |
| Intra-Run % Bias | 16.5 | 12.2 | 2.0 | 9.3 |
| n | 4 | 4 | 4 | 4 |
| Inter-Run Mean | 0.567 | 1.64 | 8.19 | 84.8 |
| Inter-Run SD | 0.0661 | 0.0509 | 0.341 | 6.21 |
| Inter-Run % CV | 11.7 | 3.1 | 4.2 | 7.3 |
| Inter-Run % Bias | 13.3 | 9.4 | 2.3 | 5.9 |
| n | 11 | 12 | 12 | 12 |

^{a}Failed to meet acceptance criteria (within ±30% from nominal concentration); excluded from statistics.

TABLE 84
Glycochenodeoxycholic Acid: Frozen (−20° C.)
Stability in Cholestyramine-Treated Human Saliva

| | | Concentration (ng/mL) | | | |
|---|---|---|---|---|---|
| Storage | Analytical | QC-LLOQ | QC-Low | QC-Mid | QC-High |
| Period | Run | 0.500 | 1.50 | 8.00 | 80.0 |
| 1 Day | 33 | 0.516 | 1.53 | 7.92 | 76.6 |
| | | 0.578 | 1.62 | 7.76 | 78.9 |
| | | 0.538 | 1.71 | 7.56 | 79.3 |
| | | 0.551 | 1.61 | 7.99 | 80.9 |
| | Mean | 0.546 | 1.62 | 7.81 | 78.9 |
| | SD | 0.0259 | 0.0737 | 0.191 | 1.77 |
| | % CV | 4.7 | 4.6 | 2.4 | 2.2 |
| | % Bias | 9.1 | 7.8 | −2.4 | −1.3 |
| | n | 4 | 4 | 4 | 4 |
| 2 Days | 33 | 0.596 | 1.39 | 7.71 | 78.3 |
| | | 0.597 | 1.48 | 8.40 | 80.4 |
| | | 0.555 | 1.51 | 8.22 | 80.3 |
| | | 0.674^{a} | 1.46 | 8.33 | 84.0 |
| | Mean | 0.583 | 1.46 | 8.17 | 80.8 |
| | SD | 0.0240 | 0.0510 | 0.312 | 2.37 |
| | % CV | 4.1 | 3.5 | 3.8 | 2.9 |
| | % Bias | 16.5 | −2.7 | 2.1 | 0.9 |
| | n | 3 | 4 | 4 | 4 |

^{a}Failed to meet acceptance criteria (within ±30% from nominal concentration); included in statistics.

TABLE 85
Glycochenodeoxycholic Acid: Matrix Effects on Quantification
(Untreated Human Saliva)

| | Concentration (ng/mL) | | | | | |
|---|---|---|---|---|---|---|
| | Cholestyramine- | | | Untreated | | |
| | Treated Human Saliva | | | Human Saliva | | |
| | QC- | QC- | QC- | | QC- | QC- |
| | Low | Mid | High | Control | Low | High |
| Analytical Run | 1.50 | 8.00 | 80.0 | Blank | 1.50 | 80.0 |
| 34 | 1.55 | 8.61 | 79.5 | BQL | 1.42 | 81.3 |
| | 1.36 | 8.30 | 81.0 | BQL | 1.33 | 78.9 |
| | 1.63 | 7.99 | 83.1 | BQL | 1.43 | 79.5 |
| | NA | NA | NA | BQL | 1.35 | 79.8 |
| | NA | NA | NA | BQL | 1.30 | 78.5 |
| | NA | NA | NA | BQL | 1.33 | 79.8 |
| Mean | 1.51 | 8.30 | 81.2 | BQL | 1.36 | 79.6 |
| SD | 0.139 | 0.310 | 1.81 | NA | 0.0529 | 0.967 |
| % CV | 9.2 | 3.7 | 2.2 | NA | 3.9 | 1.2 |
| % Bias | 0.9 | 3.7 | 1.5 | NA | −9.3 | −0.5 |
| n | 3 | 3 | 3 | 6 | 6 | 6 |

BQL Below quantitation limit (<0.500 ng/mL)
Note:
Bile acids are detected in some lots of untreated human saliva. The endogenous level for the lot used in this batch was BQL, therefore, the theoretical concentrations for QC-Low and QC-High in untreated human saliva are equal to the spiked concentrations (1.50 and 80.0 ng/mL).

TABLE 86
Glycochenodeoxycholic Acid: Long-Term (1 Month at −20° C. and
at −80° C.) Stability in Untreated Human Saliva

| | Concentration (ng/mL) | | | |
|---|---|---|---|---|
| | −20° C. Storage | | −80° C. Storage | |
| Analytical | QC-Low | QC-High | QC-Low | QC-High |
| Run | 1.50 | 80.0 | 1.50 | 80.0 |
| 35 | 1.37 | 76.9 | 1.27 | 73.0 |
| | 1.51 | 74.0 | 1.27 | 80.6 |
| | 1.46 | 77.4 | 1.34 | 77.8 |
| Mean | 1.45 | 76.1 | 1.29 | 77.1 |
| SD | 0.071 | 1.84 | 0.0404 | 3.84 |
| % CV | 4.9 | 2.4 | 3.1 | 5.0 |
| % Bias | −3.6 | −4.9 | −13.8 | −3.6 |
| n | 3 | 3 | 3 | 3 |
| Fresh QCs (Batch 34) | | | | |
| Mean at Time Zero | 1.36 | 79.6 | 1.36 | 79.6 |
| Difference from | 6.4 | −4.4 | −4.9 | −3.1 |
| Fresh (%) | | | | |

TABLE 87
Glycochenodeoxycholic Acid: Batch Acceptance Quality
Controls (Cholestyramine-Treated Human Saliva)

| | Concentration (ng/mL) | | |
|---|---|---|---|
| Analytical | QC-Low | QC-Mid | QC-High |
| Run | 1.50 | 8.00 | 80.0 |
| 35 | 2.03^{a} | 7.99 | 77.4 |
| | 1.65 | 7.97 | 86.9 |
| | 1.66 | 9.84 | 84.4 |
| Mean | 1.66 | 8.60 | 82.9 |
| SD | 0.00707 | 1.07 | 4.92 |
| % CV | 0.4 | 12.5 | 5.9 |
| % Bias | 10.3 | 7.5 | 3.6 |
| n | 2 | 3 | 3 |

^{a}Failed to meet acceptance criteria (within ±30% from nominal concentration); excluded from statistics.

TABLE 88
Taurochenodeoxycholic Acid: Calibration Standard Back-Calculated Concentrations in Cholestyramine-
Treated Human Saliva

| | Concentration (ng/mL) | | | | | | | | |
|---|---|---|---|---|---|---|---|---|---|
| | Std-1 | Std-2 | Std-3 | Std-4 | Std-5 | Std-6 | Std-7 | Std-8 | Std-9 |
| Analytical Run | 0.500 | 0.750 | 1.00 | 2.00 | 5.00 | 10.0 | 50.0 | 75.0 | 100 |
| B31 | 0.503 | 0.728 | 1.00 | 1.92 | 4.46 | 9.40 | 50.8 | 84.5 | 94.1 |
| | 0.503 | 0.782 | 1.01 | 1.97 | 4.79 | 10.1 | 48.1 | 85.6 | 102 |
| B32 | 0.499 | 0.731 | 1.01 | 2.19 | 4.91 | 9.36 | 48.7 | 76.4 | 98.6 |
| | 0.495 | 0.748 | 0.981 | 2.14 | 5.04 | 9.61 | 51.3 | 77.8 | 95.5 |
| B33 | 0.491 | 0.693 | 0.999 | 2.05 | 4.85 | 10.7 | 47.0 | 73.6 | 97.5 |
| | 0.532 | 0.766 | 0.971 | 1.96 | 4.90 | 11.4 | 45.6 | 74.6 | 107 |
| B34 | 0.516 | 0.691 | 0.992 | 1.94 | 4.94 | 9.62 | 49.5 | 74.4 | 105 |
| | 0.532 | 0.729 | 0.980 | 1.94 | 5.55 | 9.23 | 52.2 | 79.2 | 98.5 |
| B35 | 0.501 | 0.788 | 1.020 | 1.93 | 4.39 | 9.64 | 50.9 | 77.6 | 95.8 |
| | 0.506 | 0.728 | 0.966 | 2.02 | 4.83 | 10.2 | 50.0 | 84.4 | 104 |
| Mean | 0.508 | 0.738 | 0.993 | 2.01 | 4.87 | 9.93 | 49.4 | 78.8 | 99.8 |
| SD | 0.0144 | 0.0331 | 0.0180 | 0.0938 | 0.317 | 0.686 | 2.07 | 4.50 | 4.43 |
| % CV | 2.8 | 4.5 | 1.8 | 4.7 | 6.5 | 6.9 | 4.2 | 5.7 | 4.4 |
| % Bias | 1.6 | −1.5 | −0.7 | 0.3 | −2.7 | −0.7 | −1.2 | 5.1 | −0.2 |
| n | 10 | 10 | 10 | 10 | 10 | 10 | 10 | 10 | 10 |

TABLE 89
Taurochenodeoxycholic Acid: Accuracy and Precision for
Quality Controls (Cholestyramine-Treated Human Saliva)

| | Concentration (ng/mL) | | | |
|---|---|---|---|---|
| Analytical | QC-LLOQ | QC-Low | QC-Mid | QC-High |
| Run | 0.500 | 1.50 | 8.00 | 80.0 |
| 31 | 0.699^{a} | 1.70 | 9.65 | 98.0 |
| | 0.715^{a} | 1.75 | 9.91 | 104 |
| | 0.738^{a} | 1.75 | 9.97 | 92.5 |
| | 0.724^{a} | 1.74 | 9.84 | 99.0 |
| Intra-Run Mean | 0.719 | 1.74 | 9.84 | 98.4 |
| Intra-Run SD | 0.0164 | 0.0238 | 0.139 | 4.71 |
| Intra-Run % CV | 2.3 | 1.4 | 1.4 | 4.8 |
| Intra-Run % Bias | 43.8 | 15.7 | 23.0 | 23.0 |
| n | 4 | 4 | 4 | 4 |
| 32 | 0.568 | 1.74 | 8.44 | 96.4 |
| | 0.509 | 1.76 | 8.48 | 83.9 |
| | 0.570 | 1.68 | 8.29 | 92.7 |
| | 0.539 | 1.78 | 8.90 | 88.5 |
| Intra-Run Mean | 0.547 | 1.74 | 8.53 | 90.4 |
| Intra-Run SD | 0.0287 | 0.0432 | 0.261 | 5.39 |
| Intra-Run % CV | 5.3 | 2.5 | 3.1 | 6.0 |
| Intra-Run % Bias | 9.3 | 16.0 | 6.6 | 13.0 |
| n | 4 | 4 | 4 | 4 |
| 33 | 0.521 | 1.56 | 7.83 | 86.0 |
| | 0.528 | 1.61 | 7.63 | 81.6 |
| | 0.555 | 1.67 | 7.82 | 74.6 |
| | 0.561 | 1.62 | 7.62 | 96.4 |
| Intra-Run Mean | 0.541 | 1.62 | 7.73 | 84.7 |
| Intra-Run SD | 0.0197 | 0.0451 | 0.116 | 9.13 |
| Intra-Run % CV | 3.6 | 2.8 | 1.5 | 10.8 |
| Intra-Run % Bias | 8.3 | 7.7 | −3.4 | 5.8 |
| n | 4 | 4 | 4 | 4 |
| Inter-Run Mean | 0.602 | 1.70 | 8.70 | 91.1 |
| Inter-Run SD | 0.0886 | 0.0697 | 0.927 | 8.44 |
| Inter-Run % CV | 14.7 | 4.1 | 10.7 | 9.3 |
| Inter-Run % Bias | 20.5 | 13.1 | 8.7 | 13.9 |
| n | 12 | 12 | 12 | 12 |

^{a}Failed to meet acceptance criteria (within ±30% from nominal concentration); included in statistics.
QC-LLOQ fails in Batch 31

TABLE 90
Taurochenodeoxycholic Acid: Frozen (−20° C.)
Stability in Cholestyramine-Treated Human Saliva

| | | Concentration (ng/mL) | | | |
|---|---|---|---|---|---|
| Storage | Analytical | QC-LLOQ | QC-Low | QC-Mid | QC-High |
| Period | Run | 0.500 | 1.50 | 8.00 | 80.0 |
| 1 Day | 33 | 0.522 | 1.49 | 7.58 | 74.6 |
| | | 0.548 | 1.56 | 7.47 | 76.6 |
| | | 0.529 | 1.64 | 7.59 | 78.3 |
| | | 0.542 | 1.51 | 7.63 | 79.8 |
| | Mean | 0.535 | 1.55 | 7.57 | 77.3 |
| | SD | 0.0119 | 0.0668 | 0.0685 | 2.24 |
| | % CV | 2.2 | 4.3 | 0.9 | 2.9 |
| | % Bias | 7.1 | 3.3 | −5.4 | −3.3 |
| | n | 4 | 4 | 4 | 4 |
| 2 Days | 33 | 0.608 | 1.46 | 7.42 | 74.4 |
| | | 0.589 | 1.37 | 8.04 | 78.6 |
| | | 0.569 | 1.44 | 8.12 | 74.5 |
| | | 0.626 | 1.45 | 7.83 | 80.3 |
| | Mean | 0.598 | 1.43 | 7.85 | 77.0 |
| | SD | 0.0245 | 0.0408 | 0.313 | 2.97 |
| | % CV | 4.1 | 2.9 | 4.0 | 3.9 |
| | % Bias | 19.6 | −4.7 | −1.8 | −3.8 |
| | n | 4 | 4 | 4 | 4 |

TABLE 91
Taurochenodeoxycholic Acid: Matrix Effects on Quantification
(Untreated Human Saliva)

| | Concentration (ng/mL) | | | | | |
|---|---|---|---|---|---|---|
| | Cholestyramine- | | | Untreated | | |
| | Treated Human Saliva | | | Human Saliva | | |
| | QC- | QC- | QC- | | QC- | QC- |
| | Low | Mid | High | Control | Low | High |
| Analytical Run | 1.50 | 8.00 | 80.0 | Blank | 1.50 | 80.0 |
| 34 | 1.58 | 8.50 | 80.3 | BQL | 1.42 | 85.5 |
| | 1.51 | 8.41 | 83.1 | BQL | 1.38 | 82.5 |
| | 1.59 | 8.21 | 84.1 | BQL | 1.36 | 85.3 |
| | NA | NA | NA | BQL | 1.39 | 85.8 |
| | NA | NA | NA | BQL | 1.38 | 84.1 |
| | NA | NA | NA | BQL | 1.33 | 83.7 |
| Mean | 1.56 | 8.37 | 82.5 | BQL | 1.38 | 84.5 |
| SD | 0.0436 | 0.148 | 1.97 | NA | 0.0301 | 1.28 |
| % CV | 2.8 | 1.8 | 2.4 | NA | 2.2 | 1.5 |
| % Bias | 4.0 | 4.7 | 3.1 | NA | −8.2 | 5.6 |
| n | 3 | 3 | 3 | 6 | 6 | 6 |

BQL Below quantitation limit (<0.500 ng/mL)
Note:
Bile acids are detected in some lots of untreated human saliva. The endogenous level for the lot used in this batch was BQL, therefore, the theoretical concentrations for QC-Low and QC-High in untreated human saliva are equal to the spiked concentrations (1.50 and 80.0 ng/mL).

TABLE 92
Taurochenodeoxycholic Acid: Long-Term (1 Month at -20° C.
and at -80° C.) Stability in Untreated Human Saliva

| | Concentration (ng/mL) | | | |
|---|---|---|---|---|
| | −20° C. Storage | | −80° C. Storage | |
| Analytical | QC-Low | QC-High | QC-Low | QC-High |
| Run | 1.50 | 80.0 | 1.50 | 80.0 |
| 35 | 1.36 | 80.4 | 1.29 | 76.9 |
| | 1.53 | 79.1 | 1.25 | 84.4 |
| | 1.51 | 83.0 | 1.40 | 81.2 |
| Mean | 1.47 | 80.8 | 1.31 | 80.8 |
| SD | 0.0929 | 1.99 | 0.0777 | 3.76 |
| % CV | 6.3 | 2.5 | 5.9 | 4.7 |
| % Bias | −2.2 | 1.0 | −12.4 | 1.0 |
| n | 3 | 3 | 3 | 3 |
| Fresh QCs (Batch 34) | | | | |
| Mean at Time Zero | 1.38 | 84.5 | 1.38 | 84.5 |
| Difference from | 6.3 | −4.3 | −4.8 | −4.3 |
| Fresh (%) | | | | |

TABLE 93
Taurochenodeoxycholic Acid: Batch Acceptance Quality
Controls (Cholestyramine-Treated Human Saliva)

| | Concentration (ng/mL) | | |
|---|---|---|---|
| Analytical | QC-Low | QC-Mid | QC-High |
| Run | 1.50 | 8.00 | 80.0 |
| 35 | 2.07^{a} | 8.01 | 77.4 |
| | 1.68 | 8.00 | 90.8 |
| | 1.75 | 9.45 | 85.8 |
| Mean | 1.72 | 8.49 | 84.7 |
| SD | 0.0495 | 0.834 | 6.77 |
| % CV | 2.9 | 9.8 | 8.0 |
| % Bias | 14.3 | 6.1 | 5.8 |
| n | 2 | 3 | 3 |

^{a}Failed to meet acceptance criteria (within ±30% from nominal concentration); excluded from statistics.

### Example 4: Saliva Study

Results of the saliva study, as performed by the methods disclosed in Examples 1-3, are summarized. This set of studies has the following objectives: determine if bile acid measurement in the saliva assay study correlate with Bilitec results; determine if patients with bile acid positive measurements taken from saliva samples respond to IW-3718. The data suggest that there is a spike in salivary bile acid 1-2 hours after a meal. Bile Acids in saliva from cholestatic patients were shown to be 1-2% of that in serum. Serum average range in healthy volunteers is 2-10 μM with a 2-5× increase after a meal (peak 60-90 min post-prandial). Note that persistent GERD patients (patients who do not respond to PPI) are not captured in this study.

The LC/MS/MS method used in Example 1-3 can quantitate 10 bile acids simultaneously and has a 0.001 μmol/L limit of detection. The commercially available Colorimetric kit is designed for serum or cell lysates and is not sensitive enough for saliva (could not detect bile acid; i.e., limit of detection not low enough).

Correlation with Bilitec

Analysis can only be done with the subjects that had Bilitec and saliva sampling (n=45). Analysis includes screen fails (<3 time points collected): 31.

Saliva definitions: Timepoints used for saliva bile acid assessment are screening, pre-treatment, and randomization. Saliva negative: all samples prior to dosing (up to 3) are under threshold. Saliva positive: any 1 sample is above the threshold.

There is a trending correlation between saliva bile acids (BA) measurements with Bilitec.

64% agreement: 29/45

45 patients in Bilitec group

29 Bilitec agreements: 16 Negative and 13 Positive

16 Bilitec disagreements

10 Bilitec positive that are saliva BA negative.

8/10 patients are screen failures and all samples >2 h post-meal.

2/10 patients were study completers. All timepoints >5 h post-meal.

6 Bilitec negative with positive saliva (false positives with respect to saliva?).

3/6 patients were originally Bilitec positive and switched at final reading.

2/6 patients not switched had saliva collection within the optimal timeframe.

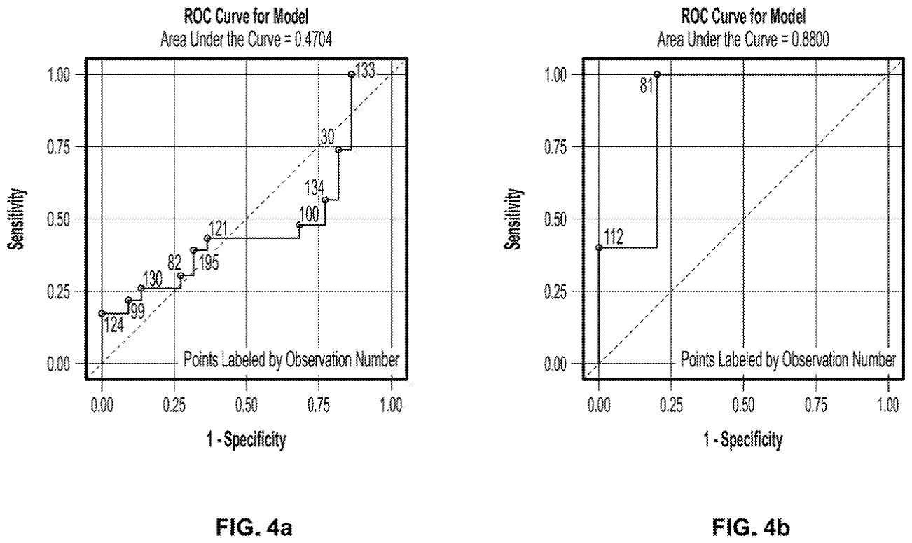

FIG. 4 shows graphically that when sampling saliva is within (less than or equal to) 2 hours post meal, correlation is seen between saliva bile acid positive and Bilitec positive results. FIG. 4A: ROC curve for all 45 subjects with Bilitic, AUC=0.47. FIG. 4B: ROC curve for all 10 subjects with saliva sampling within 2 hours post meal; AUC=0.88.

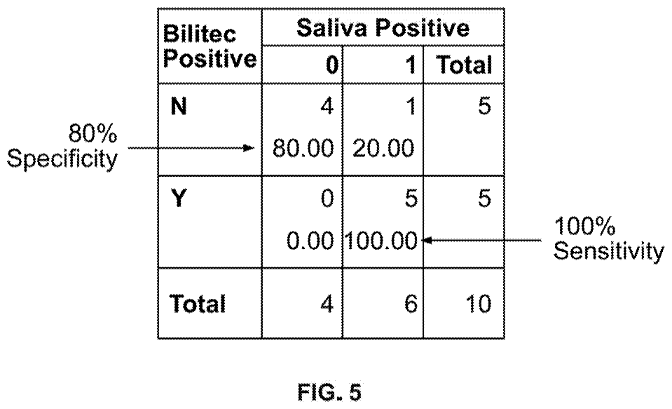

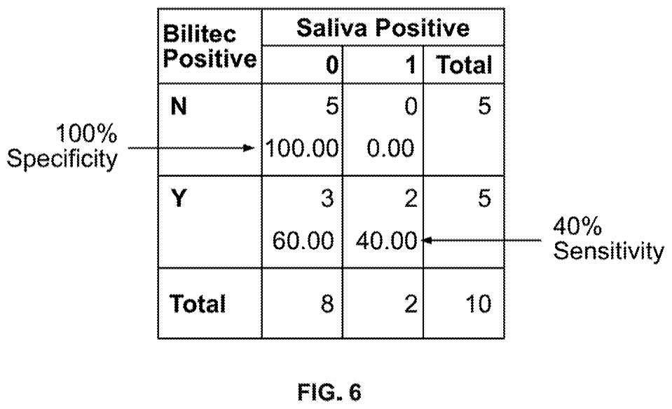

Using saliva samples collected within 2 hours post meal, the threshold to reach 100% sensitivity is 13 nM. See FIG. 5. Using saliva samples collected ≤2 hrs post meal, the threshold to reach 100% specificity is 37 nM. See FIG. 6.

Predicting Treatment Outcome

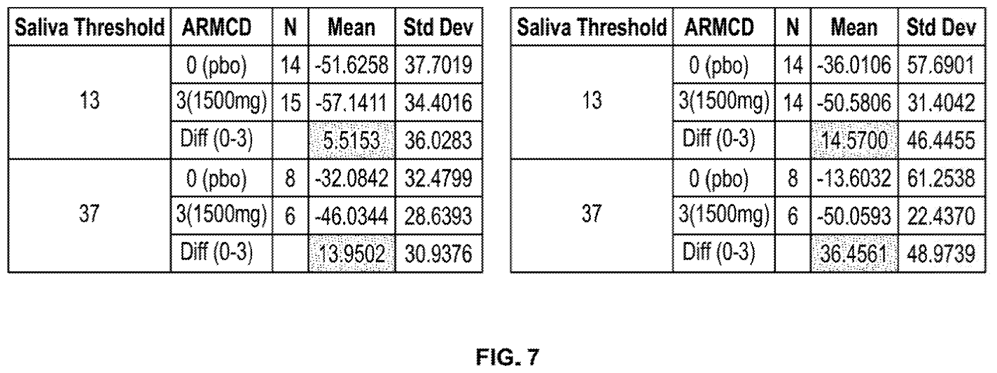

Efficacy results in saliva bile positive subgroup compared with overall population is shown in FIG. 7. There is better differentiation at saliva threshold of 37, but small sample numbers.

Unconjugated bile acids used in vitro/ex vivo demonstrate LES relaxation and increase the expression of biomarker of esophageal tissue injury. Deoxycholic acid (DCA) is the most abundant unconjugated bile acid.

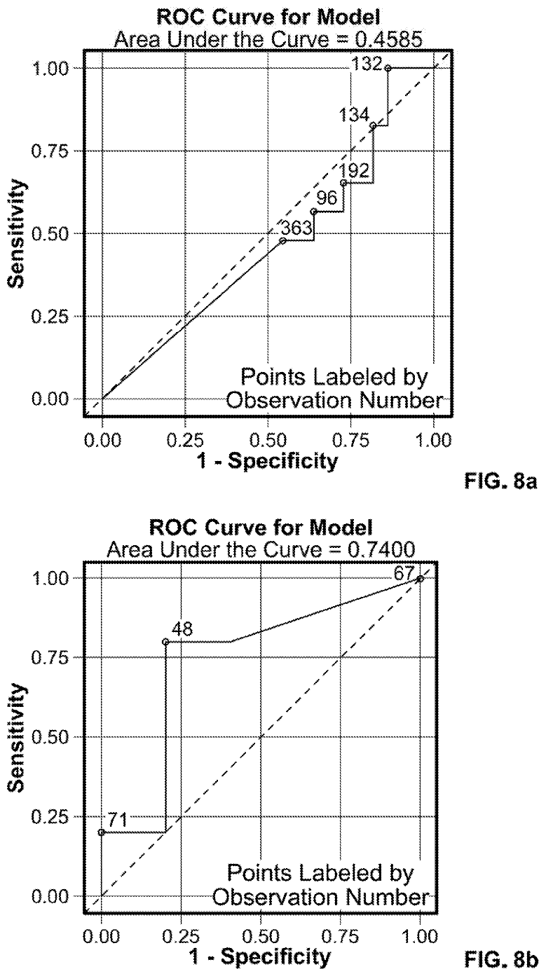

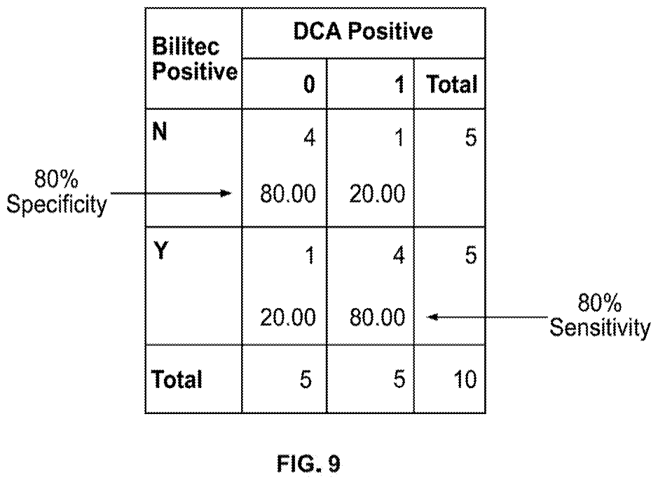

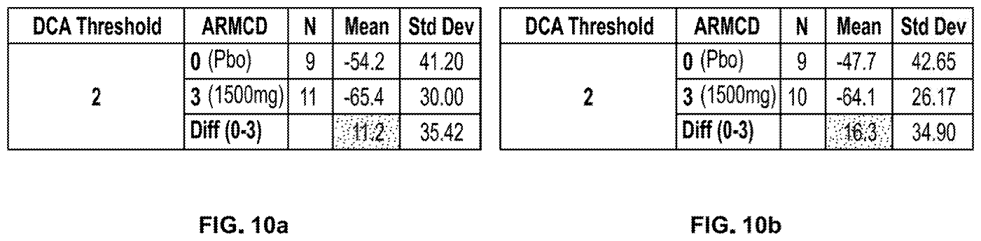

When sampling saliva within 2 hours post meal, correlation is seen between saliva bile DCA positive and Bilitec positive results, similar trend to total bile acids. See FIG. 8. Using saliva samples collected ≤2 hrs post meal, the DCA threshold to reach 80% sensitivity/specificity is 2 nM. See FIG. 9. Efficacy results in saliva bile DCA positive subgroup compared with the overall population are shown in FIG. 10.

### CONCLUSIONS

There is a correlation between saliva BA measurements with Bilitec results using LC-MS/MS method. The correlation analysis of unconjugated bile acid, DCA, with Bilitec showed similar trend to analysis of total bile acids. Test of bile acid in saliva samples taken within 2 hours post meal appears to identify bile reflux patients. Limited data point to 13 nM as the potential saliva bile acid positive threshold for 100% sensitivity and 37 nM for 100% specificity. Saliva bile acid exceeding a certain threshold (e.g., 37) may predict better treatment outcomes.

### OTHER EMBODIMENTS

The foregoing description discloses only exemplary embodiments of the invention.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the appended claims. Thus, while only certain features of the invention have been illustrated and described, many modifications and changes occur to those skilled in the art. It is therefore to be understood that the appended claims are intended to cover all such modifications and changes as fall within the true spirit of the invention.

What is claimed is:

1. A method of detecting and quantifying bile acids from saliva from a human patient, comprising: collecting saliva from said patient and determining the bile acid levels in the saliva using liquid chromatography with tandem mass spectrometry.

2. The method of claim 1, wherein the human patient is being treated with an enteric coated gastro-retentive oral dosage form in the form of a tablet of a bile acid sequestrant dispersed in a polymeric matrix.

3. The method of claim 2, wherein the polymeric matrix comprises polyethylene oxide CAS Number 25322-68-3, approximate molecular weight 300,000 (Polyox™ WSR N-750 (INCI name PEG-7M)).

4. The method of any one of claims 1-3, wherein the dosage form further comprises one or more filler or compressing agent.

5. The method of claim 4, wherein the one or more filler or compressing agent is selected from microcrystalline cellulose, butylated hydroxytoluene, colloidal silicon dioxide, lactose, starch, maltodextrins, magnesium stearate, diacetylated monoglycerides, hypromellose, and dibasic calcium phosphate.

6. The method of any one of the preceding claims, wherein the tablet is coated with an enteric coating.

7. The method of any one of the preceding claims, further comprising administering a pharmaceutical composition comprising a proton pump inhibitor (PPI).

8. The method of any one of the preceding claims, wherein the bile acid sequestrant is colesevelam or colesevelam hydrochloride.

9. The method of any one of the preceding claims, wherein the patient is administered a dose of 500 mg, 700 mg, 750 mg, 1,000 mg, 1400 mg, 1,500 mg, or 2,100 mg, or more, of the bile acid sequestrant, twice per day.

10. The method of any one of the preceding claims, wherein the patient is administered a dose is 1,500 mg, twice per day.

11. The method of claim 10, wherein the dose of 1,500 mg is administered as either 2 tablets, each tablet having 750 mg of the bile acid sequestrant or as 3 tablets, each tablet having 500 mg of the bile acid sequestrant, twice per day.

12. The method of any one of the preceding claims, wherein the saliva sample has a concentration of total bile acids exceeding 50 μmol/L.

13. The method of any one of the preceding claims, wherein the saliva sample has a concentration of total bile acids exceeding 13 nM.

14. The method of any one of the preceding claims, wherein the saliva sample has a concentration of total bile acids exceeding 37 nM.
